(19) United States
(12) Patent Application Publication
(10) Pub. No.: US 2026/0337327 A1
(43) Pub. Date: Oct. 29, 2026

(54) BIOACTIVE PEPTIDE BRUSH POLYMERS VIA PHOTOINDUCED REVERSIBLE-DEACTIVATION RADICAL POLYMERIZATION

(71) Applicant: Northwestern University, Evanston, IL (US)

(72) Inventors: Nathan C. GIANNESCHI, Evanston, IL (US); Hao SUN, Evanston, IL (US)

(73) Assignee: Northwestern University, Evanston, IL (US)

(21) Appl. No.: 19/406,568

(22) Filed: Dec. 2, 2025

Related U.S. Application Data
(63) Continuation of application No. 17/764,746, filed on Sep. 29, 2020, now Pat. No. 12,516,145.
(60) Provisional application No. 62/907,993, filed on Sep. 30, 2019.
(60) Provisional application No. 63/050,277, filed on Jul. 10, 2020.

(51) Int. Cl. C08F 299/02; C07K 1/107; C08F 2/06; C08F 2/10; C08F 2/50; C08F 220/56

(52) U.S. Cl. CPC C08F 299/022; C07K 1/1077; C08F 2/06; C08F 2/10; C08F 2/50; C08F 220/56; C08F 2438/03

(57) ABSTRACT

Aspects of the invention include a method for synthesizing a peptide brush polymer, the method comprising: exposing a mixture comprising peptide-containing monomers, one or more photoinitiators, and one or more chain transfer agents to a light sufficient to induce photopolymerization, and photopolymerizing the peptide-containing monomers in the mixture; wherein: the resulting peptide brush polymer comprises at least one peptide-containing polymer block; the at least one peptide-containing polymer block is characterized by a degree of polymerization of at least 10 and a peptide graft density of 50% to 100%; and at least one peptide moiety of the at least one peptide-containing polymer block has 5 or more amino acid groups.

### CROSS-REFERENCE TO RELATED APPLICATIONS

This application is a continuation of U.S. patent application Ser. No. 17/764,746, filed Mar. 29, 2022, which is a U.S. National Stage Application under 35 U.S.C. § 371 of International Application No. PCT/US2020/053308, filed Sep. 29, 2020, which claims the benefit of priority to U.S. Provisional Patent Applications No. 62/907,993, filed Sep. 30, 2019, and No. 63/050,277, filed Jul. 10, 2020, each of which is hereby incorporated by reference in its entirety.

### STATEMENT REGARDING FEDERALLY SPONSORED RESEARCH OR DEVELOPMENT

This invention was made with government support under award number R01HL139001 awarded by the National Institutes of Health and the National Heart Lung and Blood Institute, under award number DMR-1710105 awarded by the National Science Foundation, and under Grant No. FA9550-16-1-0150 awarded by the Air Force Office of Scientific Research (AFOSR). The government has certain rights in the invention.

### REFERENCE TO A SEQUENCE LISTING

A sequence listing containing SEQ ID NOs.: 1-12, created Dec. 1, 2025, size: 16,469 bytes, and titled “340495_134-19A_NU2019-180-04_seq_list_ST26.xml”, is provided herewith in a computer-readable nucleotide/amino acid .xml file and is specifically incorporated by reference.

### BACKGROUND OF INVENTION

Brush polymers that include peptides as side chain have the potential to provide enhanced biological activities and cell penetration, leading to new or improved therapies. Such polymers could be made by a variety of techniques, including peptide brush polymers prepared via ring-opening metathesis polymerization (ROMP) and atom transfer radical polymerization. However, such methods involve the use heavy metals such as ruthenium and copper-based catalysts. The possibility of residual heavy metals remaining after synthesis raises concerns for biomedical applications.

Accordingly, there exists a need in the for metal-residue-free brush polymers with bioactive peptides. Additional desirable properties and synthesis parameters would include: the ability to use mild reaction conditions, retain spatiotemporal control over the reaction with minimal if any additional reactive molecules, room temperature reaction, aqueous reactions, the polymerization's high tolerance of oxygen and water, high degree of functionalization or peptide graft density, high bioactivity and cellular uptake efficiency, and a controllable degree of toxicity according to therapeutic needs.

### SUMMARY OF THE INVENTION

Provided herein are peptide-containing brush polymers (peptide brush polymers), and methods for synthesizing these, that provide the above mentioned advantages. These synthesis methods describes herein use photo-induced polymerization and can be performed in aqueous solutions, in organic conditions, at room temperature, and with exposure to oxygen. The methods disclosed herein provide for well-controlled peptide graft density and brush architecture, leading to a well-controlled bioactivity, such as to tune cellular uptake and cytotoxicity according to the needs of a biomedical application. For example, certain peptides described herein feature enzyme-responsive and pro-apoptotic amino acid sequences. While copolymerization with comonomers provide advantages in some embodiments, these methods also provide for synthesis of peptide brush polymers with 100% peptide graft density, for example to maximize bioactivity and synthesis efficiency (no need for comonomers and complex array of copolymer architectures, in some embodiments).

Aspects of the invention include a method for synthesizing a peptide brush polymer, the method comprising: exposing a mixture comprising peptide-containing monomers, one or more photoinitiators, and one or more chain transfer agents to a light, and photopolymerizing the peptide-containing monomers in the mixture; wherein: the resulting peptide brush polymer comprises at least one peptide-containing polymer block; the at least one peptide-containing polymer block is characterized by a degree of polymerization of at least 10 and a peptide graft density of 50% to 100%; and at least one peptide moiety of the at least one peptide-containing polymer block has 5 or more amino acid groups. The light, to which the mixture is exposed, should be sufficient or capable of inducing or initiating the photopolymerization of the peptide-containing monomers (and preferably other monomers in the mixture) in the presence of said mixture. Preferably for some applications, in any of the methods and polymers disclosed herein, at least one peptide moiety of the at least one peptide-containing polymer block has at least 6 amino acid groups, preferably for some applications at least 7 amino acid groups, preferably for some applications at least 8 amino acid groups, preferably for some applications at least 9 amino acid groups, preferably for some applications at least 10 amino acid groups, preferably for some applications at least 11 amino acid groups, preferably for some applications at least 12 amino acid groups, preferably for some applications at least 13 amino acid groups, preferably for some applications at least 14 amino acid groups, preferably for some applications at least 15 amino acid groups. Optionally, in any of the methods and polymers disclosed herein, each peptide moiety (or, “Pep” in formulas below) of the peptide brush polymer independently has at least 5 amino acids, preferably for some applications at least 6 amino acid groups, preferably for some applications at least 7 amino acid groups, preferably for some applications at least 8 amino acid groups, preferably for some applications at least 9 amino acid groups, preferably for some applications at least 10 amino acid groups, preferably for some applications at least 11 amino acid groups, preferably for some applications at least 12 amino acid groups, preferably for some applications at least 13 amino acid groups, preferably for some applications at least 14 amino acid groups, preferably for some applications at least 15 amino acid groups.

Preferably, in any of the methods and polymers disclosed herein, the peptide-containing monomers are photopolymerized according a monomer conversion of greater than or equal to 90%, preferably greater than or equal to 95%, more preferably greater than or equal to 98%, more preferably greater than or equal to 99%. Preferably, in any of the methods and polymers disclosed herein, the mixture has a temperature selected from the range of 5 °C to 45 °C during the photopolymerizing step, optionally 5 °C to 40 °C, optionally 5 °C to 35 °C, optionally 10 °C to 45 °C, optionally 10 °C to 40 °C, optionally 10 °C to 35 °C, optionally 10 °C to 30 °C, optionally 15 °C to 30 °C, optionally 15 °C to 25 °C, more preferably 20 °C to 25 °C. Optionally, in any of the methods and polymers disclosed herein, the mixture is exposed to an inert or non-oxygen gas(es) during the photopolymerizing step. Optionally, in any of the methods and polymers disclosed herein, the mixture is exposed to nitrogen gas and/or argon gas during the photopolymerizing step. Optionally, in any of the methods and polymers disclosed herein, the mixture is aqueous. Optionally, in any of the methods and polymers disclosed herein, the light is characterized by wavelength(s) selected from the range of about 320 nm to about 700 nm, during the photopolymerizing step, selected from the range of about 320 nm to about 500 nm, optionally selected from the range of about 345 nm to about 405 nm, optionally selected from the range of about 410 nm to about 500 nm. The light can be coherent, noncoherent, semicoherent, or any combination of these.

Optionally, in any of the methods and polymers disclosed herein, the one or more photoinitiators comprise eosin Y disodium, pentamethyldiethylenetriamine, sodium phenyl-2,4,6-trimethylbenzoylphosphinate, lithium phenyl-2,4,6-trimethylbenzoylphosphinate, Zn(II) meso-Tetra(4-sulfonatophenyl) porphine, or a combination of these. Optionally, in any of the methods and polymers disclosed herein, the one or more photoinitiators comprise eosin Y disodium, pentamethyldiethylenetriamine, sodium phenyl-2,4,6-trimethylbenzoylphosphinate, lithium phenyl-2,4,6-trimethylbenzoylphosphinate, Zn(II) meso-Tetra(4-sulfonatophenyl) porphine, any substituted form of these, any derivative of these, or a combination of these. Optionally, in any of the methods and polymers disclosed herein, each of the one or more chain transfer agents comprises one or more trithiocarbonate groups, one or more carboxylic acids, or any combination of these. Optionally, in any of the methods and polymers disclosed herein, the one or more chain transfer agents comprises a chain transfer agent characterized by formula FX13:

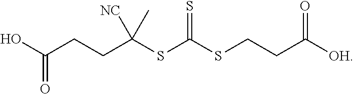 [SMILES: CC(C#N)(CCC(=O)O)SC(=S)SCCC(=O)O]

Optionally, in any of the methods and polymers disclosed herein, the one or more chain transfer agents are water-soluble. Optionally, in any of the methods and polymers disclosed herein, the mixture further comprises at least one comonomer, each comonomer being free of a peptide sequence; and wherein the photopolymerizing step comprises copolymerizing the peptide-containing monomers and the at least one comonomer.

Preferably, in any of the methods and polymers disclosed herein, the at least one peptide-containing polymer block is characterized by a peptide graft density of 70% to 100%, more preferably 80% to 100%, more preferably 90% to 100%, further more preferably 92% to 100%, further more preferably 94% to 100%, further more preferably 95% to 100%, further more preferably 96% to 100%, further more preferably 98% to 100%, further more preferably 99% to 100%. The method of any one of the preceding claims, wherein the at least one peptide-containing polymer block is characterized by a peptide graft density of 100%. Preferably, in any of the methods and polymers disclosed herein, the at least one peptide-containing polymer block is bound to a second polymer block, wherein the second polymer block has a peptide graft density of 0% to 100%. Optionally, in any of the methods and polymers disclosed herein, the at least one peptide-containing polymer block is bound to a second polymer block, wherein the second polymer block has a peptide graft density of 0% to 50%, optionally 50% to 100%, optionally 75% to 100%, optionally 0% to 25%, optionally 0% to 10%, optionally 0% to 5%, optionally 0%.

Optionally, in any of the methods and polymers disclosed herein, the method comprises copolymerizing a second polymer block with the at least one peptide-containing polymer. Optionally, in any of the methods and polymers disclosed herein, the second polymer block is hydrophobic. Optionally, in any of the methods and polymers disclosed herein, the step of copolymerizing the second polymer block is performed after the step of photopolymerizing the at least one peptide-containing block.

Optionally, in any of the methods and polymers disclosed herein, the step of copolymerizing the second polymer block comprises a photopolymerization. Preferably, in any of the methods and polymers disclosed herein, the method further comprises isolating the peptide brush polymer. Optionally, in any of the methods and polymers disclosed herein, the method comprises removing substantially all unreacted monomers, photoinitiators, and chain transfer agents, after the step of photopolymerizing. Optionally, in any of the methods and polymers disclosed herein, the resulting peptide brush polymer forms a micelle or nanoparticle.

Optionally, in any of the methods and polymers disclosed herein, the method comprises comprising self-assembly of the peptide brush polymer into a micelle or nanoparticle. Optionally, in any of the methods and polymers disclosed herein, the method comprises dispersing the peptide brush polymer in water or an aqueous solution. Optionally, in any of the methods and polymers disclosed herein, each monomer, each chain transfer agent, each photoinitiator, and the resulting peptide brush polymer are metal-free.

Optionally, in any of the methods and polymers disclosed herein, the method comprises metal-free photoinduced reversible-deactivation radical polymerization and/or photo-electron transfer reversible addition-fragmentation transfer polymerization. Optionally, in any of the methods and polymers disclosed herein, the method comprises exposing the peptide brush polymer to an enzyme and causing enzymatic digestion of at least a portion of the peptide brush polymer. Optionally, in any of the methods and polymers disclosed herein, the method comprises administering to a subject an effective amount of the peptide brush polymer to treat or manage a condition.

Optionally, in any of the methods and polymers disclosed herein, each peptide-containing monomer in the mixture has a peptide sequence that is the same. Optionally, in any of the methods and polymers disclosed herein, the peptide brush polymer comprises at least two different peptide sequences. Optionally, in any of the methods and polymers disclosed herein, the peptide brush polymer comprises at least three different peptide sequences. Optionally, in any of the methods and polymers disclosed herein, the peptide brush polymer comprises at least four different peptide sequences, optionally at least 5 different peptide sequences, optionally at least 6 different peptide sequences.

Optionally, in any of the methods and polymers disclosed herein, each peptide-containing monomer is independently characterized by formula FX1: Z-(A-Pep)ₓ (FX1); wherein: Z is a polymer backbone precursor group; A is a covalent anchor group; Pep is a peptide moiety; and x is an integer selected from the range of 1 to 2. Optionally, in any of the methods and polymers disclosed herein, Z comprises an olefin group, a vinyl group, an acrylate group, an acrylamide group, a styrene group, or any combination of these. Optionally, in any of the methods and polymers disclosed herein, Z comprises an olefin group, a vinyl group, an acrylate group, an acrylamide group, a styrene group, an aryl group, a cycloalkenyl group, a cycloalkenylene group, an alkene group, or any combination of these. Optionally, in any of the methods and polymers disclosed herein, Z comprises an olefin group, a vinyl group, an acrylate group, an acrylamide group, or any combination of these. Optionally, in any of the methods and polymers disclosed herein, Z does not comprise a ROMP-polymerizable group. Optionally, in any of the methods and polymers disclosed herein, Z does not comprise a norbornene group.

Optionally, in any of the methods and polymers disclosed herein, Z is characterized by formula FX2A, FX2B, FX2C, FX2D, FX2E, or FX2F:

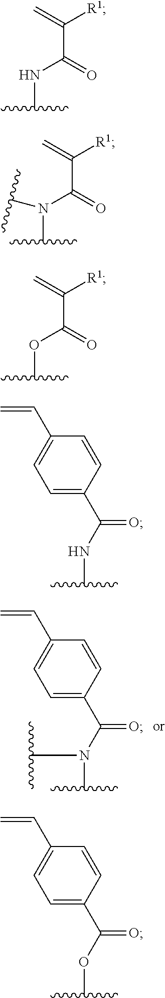

wherein: R¹ is a hydrogen or a methyl group. Optionally, in any of the methods and polymers disclosed herein, each A independently selected from the group consisting of single bond, an oxygen, and one or more substituted or substituted groups having an alkyl group, an alkenylene group, an arylene group, an alkoxy group, an acyl group, a carboxyl group, an aliphatic group, an amide group, an aryl group, an amine group, an ether group, a ketone group, an ester group, a triazole group, a diazole group, a pyrazole group, or combinations thereof. Optionally, in any of the methods and polymers disclosed herein, each A is independently characterized by formula FX3A, FX3B, or FX3C;

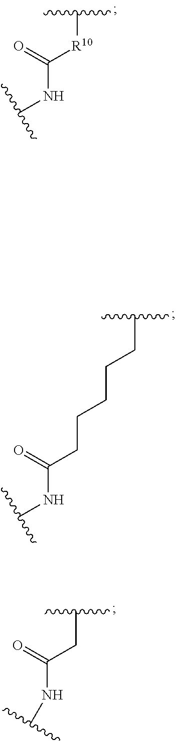

wherein: R¹⁰ is a substituted or unsubstituted C1-C10 alkyl. Optionally, in any of the methods and polymers disclosed herein, each Pep comprises at least 5 amino acids, preferably for some applications at least 6 amino acids, preferably for some applications at least 7 amino acids, preferably for some applications at least 8 amino acids, preferably for some applications at least 9 amino acids, preferably for some applications at least 10 amino acids, more preferably for some applications at least 11 amino acids, more preferably for some applications at least 12 amino acids, more preferably for some applications at least 15 amino acids.

Optionally, in any of the methods and polymers disclosed herein, each P comprises a sequence having at least 80% sequence homology with SEQ ID NO:1 (GPLGLAGGWGERDGS), SEQ ID NO:2 (GALTPRGADSGSG), SEQ ID NO:3 (KLAKLAKKLAKLAK), SEQ ID NO:4 (GSGKEFGADSGSG), SEQ ID NO:5 (GPLGLAGG), SEQ ID NO:6 (HVLVMSATKKKK), SEQ ID NO:7 (GGGCYFQNCPKG) (Terlipressin), SEQ ID NO:8 (DRVYIHPF) (Angiotensin 2), SEQ ID NO: 9 (AQYQDKLAR) (DA1), SEQ ID NO:10 (GVi(allo)SQIRP) (ABT898), SEQ ID NO:11 (KVPRNQDWL) (gp100), SEQ ID NO:12 (GPLGLAGGWGER), or a combination of these.

Optionally, in any of the methods and polymers disclosed herein, each comonomer, if present, in the mixture is independently characterized by formula FX4:

Z′-(M)_{y} (FX4); wherein: Z′ is a polymer backbone precursor group; M is an alkyl group, an alkenylene group, an arylene group, an alkoxy group, an acyl group, a carboxyl group, an aliphatic group, an amide group, an aryl group, an amine group, an ether group, a ketone group, an ester group, or combinations thereof; and y is an integer selected from the range of 1 to 2. Optionally, in any of the methods and polymers disclosed herein, each comonomer, if present, in the mixture is independently characterized by formula FX5A, FX5B, FX5C:

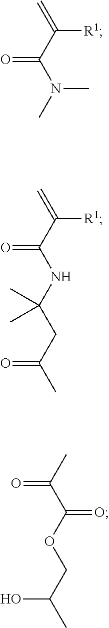

wherein: R¹ is a hydrogen or a methyl group.

Optionally, in any of the methods and polymers disclosed herein, the peptide brush polymer is characterized by formula FX6A or FX6B:

Q¹-[B¹]ₘ-Q² (FX6A); or Q¹-[B¹]ₘ−/−[B²]ₙ-Q² (FX6B); wherein: each B¹ is independently a peptide-containing polymer block; each B² is independently a peptide-free polymer block; each of m and n is independently an integer greater than or equal to 1; the symbol “/” indicates that the units separated thereby are covalently linked randomly or in any order; and each of Q¹ and Q² is independently a polymer terminating group.

Optionally, in any of the methods and polymers disclosed herein, each B¹ is characterized by the formula (FX7):

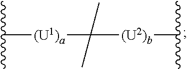

wherein: each U¹ is independently a peptide-containing repeating unit; each U² is independently a peptide-free repeating unit; a is an integer selected from the range of 2 to 500, preferably 2 to 100; b is 0 or an integer selected from the range of 2 to 500, preferably 2 to 100; and the symbol “/” indicates that the units separated thereby are covalently linked randomly or in any order. Optionally, in any of the methods and polymers disclosed herein, each U¹ is independently characterized by the formula FX8A or FX8B and each U², if present, is independently characterized by the formula FX9A or FX9B:

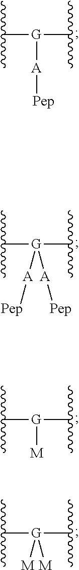

wherein: each G is independently a polymer backbone group; each A is independently a covalent anchor group; each Pep is independently a peptide moiety; and each M is independently an alkyl group, an alkenylene group, an arylene group, an alkoxy group, an acyl group, a carboxyl group, an aliphatic group, an amide group, an aryl group, an amine group, an ether group, a ketone group, an ester group, or combinations thereof. Optionally, in any of the methods and polymers disclosed herein, each G is independently characterized by formula FX10A, FX10B, FX10C, FX10D, FX10E, or FX10F:

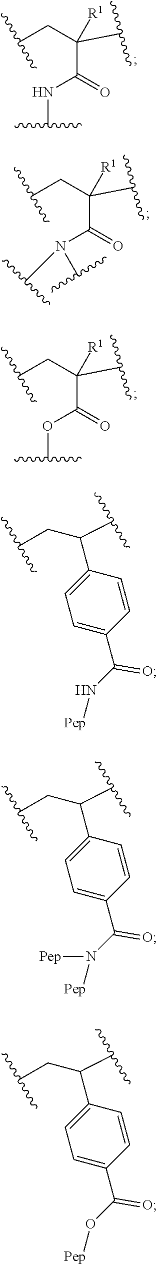

wherein: R¹ is a hydrogen or a methyl group. Optionally, in any of the methods and polymers disclosed herein, the peptide brush polymer is characterized by formula FX6A and m is 1. Optionally, in any of the methods and polymers disclosed herein, the peptide brush polymer is characterized by formula FX11A or FX11B:

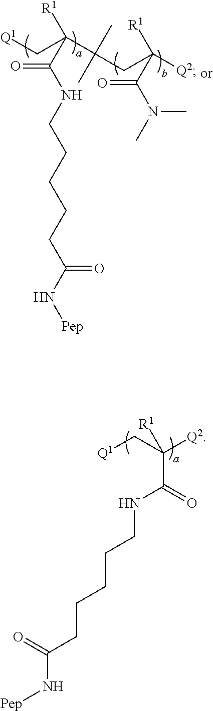

Optionally, in any of the methods and polymers disclosed herein, the peptide brush polymer is characterized by formula FX6A, m is 1, and b is 0. Optionally, in any of the methods and polymers disclosed herein, the peptide brush polymer is characterized by formula FX6A, m is 1, b is 0, and a is an integer selected from the range of 10 to 100. Optionally, in any of the methods and polymers disclosed herein, each of Q¹ and Q² is independently a hydrogen or characterized by formula FX14A or FX14B:

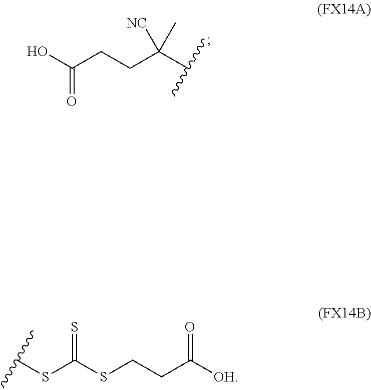

Optionally, in any of the methods and polymers disclosed herein, Q¹ is characterized by formula FX14A. Optionally, in any of the methods and polymers disclosed herein, Q² is hydrogen or characterized by formula FX14B. Optionally, in any of the methods and polymers disclosed herein, the peptide brush polymer is characterized by formula FX12:

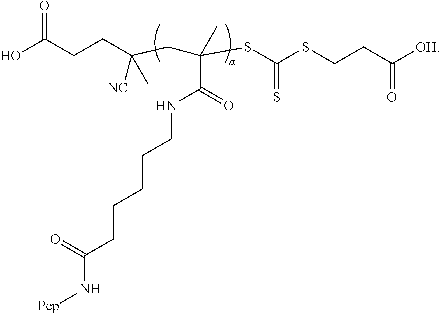

Optionally, in any of the methods and polymers disclosed herein, each peptide moiety (or, “Pep” in formulas) of the peptide brush polymer independently has at least 5 amino acids, preferably for some applications at least 6 amino acid groups, preferably for some applications at least 7 amino acid groups, preferably for some applications at least 8 amino acid groups, preferably for some applications at least 9 amino acid groups, preferably for some applications at least 10 amino acid groups, preferably for some applications at least 11 amino acid groups, preferably for some applications at least 12 amino acid groups, preferably for some applications at least 13 amino acid groups, preferably for some applications at least 14 amino acid groups, preferably for some applications at least 15 amino acid groups.

Optionally, in any of the methods and polymers disclosed herein, the at least one peptide-containing polymer block is hydrophilic. Optionally, in any of the methods and polymers disclosed herein, in the peptide brush polymer comprises a hydrophobic peptide-free polymer block.

Optionally, in any of the methods and polymers disclosed herein, the peptide brush polymer is water-soluble. Optionally, in any of the methods and polymers disclosed herein, the peptide brush polymer is amphiphilic. Optionally, in any of the methods and polymers disclosed herein, the peptide brush polymer is in the form of a micelle or nanoparticle. Optionally, in any of the methods and polymers disclosed herein, the peptide brush polymer is provided in an aqueous solution and wherein the peptide brush polymer is in the form of a micelle or nanoparticle in said aqueous solution. Optionally, in any of the methods and polymers disclosed herein, each peptide moiety or Pep is a branched polypeptide, a linear polypeptide or a cross-linked polypeptide. Optionally, in any of the methods and polymers disclosed herein, each peptide moiety or Pep is a therapeutic peptide. Preferably, in any of the methods and polymers disclosed herein, each of at least 30%, preferably at least 50%, preferably at least 70%, more preferably at least 80%, further more preferably at least 90%, further more preferably all of the peptide moieties is a therapeutic peptide. Preferably, in any of the methods and polymers disclosed herein, any of the peptide sequences of the peptide brush polymer have a higher bioactivity and higher cellular uptake efficiency compared to the same peptide sequence provided in absence of the peptide brush polymer. Preferably, in any of the methods and polymers disclosed herein, each of at least 50% of the peptide sequences of the peptide brush polymer have a higher bioactivity and higher cellular uptake efficiency compared to the same peptide sequences, respectively, provided in absence of the peptide brush polymer.

Preferably, in any of the methods and polymers disclosed herein, the peptide brush polymer is characterized by a solubility in water of at least 50 mg/mL, preferably at least 100 mg/mL, more preferably at least 150 mg/mL, further more preferably at least 200 mg/mL, optionally selected from the range of 50 mg/mL to 200 mg/mL, optionally selected from the range of 100 mg/mL to 200 mg/mL.

Aspects of the invention include a peptide brush polymer formed from any of the embodiments, or combinations thereof, described herein. Aspects of the invention include a peptide brush polymer comprising: at least 5 peptide-containing repeating units; wherein each peptide-containing repeating unit comprises a poly(meth)acrylamide or poly(meth)acrylate polymer backbone group directly or indirectly covalently linked to a polymer side chain group comprising a peptide moiety; wherein: the peptide brush polymer is characterized by a degree of polymerization of at least 10 and a peptide graft density of 50% to 100%; and each peptide moiety has at least 10 amino acid groups. Optionally, in any of the methods and polymers disclosed herein, the polymer is characterized by a degree of polymerization of at least 15. Optionally, in any of the methods and polymers disclosed herein, the polymer has a peptide graft density of 90% to 100%. Optionally, in any of the methods and polymers disclosed herein, the polymer has a peptide graft density of 100%. Optionally, in any of the methods and polymers disclosed herein, each peptide moiety has at least 6 amino acid groups, preferably at least 7 amino acid groups, preferably at least 8 amino acid groups, preferably at least 9 amino acid groups, preferably at least 10 amino acid groups, preferably at least 11 amino acid groups, preferably at least 12 amino acid groups, preferably at least 13 amino acid groups, preferably at least 14 amino acid groups, preferably at least 15 amino acid groups. Optionally, in any of the polymers disclosed herein, the at least one peptide-containing polymer block is hydrophilic. Optionally, in any of the polymers disclosed herein, the peptide brush polymer comprises a hydrophobic peptide-free polymer block. Optionally, in any of the polymers disclosed herein, the peptide brush polymer is water-soluble. Optionally, in any of the polymers disclosed herein, the peptide brush polymer is amphiphilic. Optionally, in any of the polymers disclosed herein, the peptide brush polymer is in the form of a micelle or nanoparticle. Optionally, in any of the polymers disclosed herein, the peptide brush polymer is provided in an aqueous solution and wherein the peptide brush polymer is in the form of a micelle or nanoparticle in said aqueous solution. Optionally, in any of the polymers disclosed herein, each peptide moiety or Pep is a branched polypeptide, a linear polypeptide or a cross-linked polypeptide. Optionally, in any of the polymers disclosed herein, each of at least 50% of the peptide moieties is a therapeutic peptide.

Optionally, in any of the methods and polymers disclosed herein, the peptide brush polymer is characterized by formula FX13A:

Q¹-[U¹]ₐ−/−[U²]_{b}-Q² (FX13A); or each of Q¹ and Q² is independently a polymer terminating group; each U¹ is independently a peptide-containing repeating unit; each U² is independently a peptide-free repeating unit; a is an integer selected from the range of 2 to 500, preferably 2 to 100; b is 0 or an integer selected from the range of 2 to 500, preferably 2 to 100; and the symbol “/” indicates that the units separated thereby are linked randomly or in any order. Optionally, in any of the methods and polymers disclosed herein, each U¹ is independently characterized by the formula FX8A or FX8B and each U², if present, is independently characterized by the formula FX9A or FX9B:

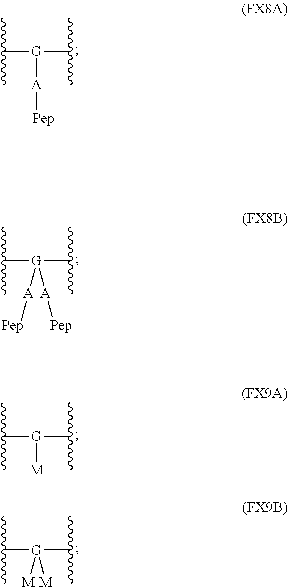

wherein: each G is independently a polymer backbone group; each A is independently a covalent anchor group; each Pep is independently a peptide moiety; and each M is independently an alkyl group, an alkenylene group, an arylene group, an alkoxy group, an acyl group, a carboxyl group, an aliphatic group, an amide group, an aryl group, an amine group, an ether group, a ketone group, an ester group, or combinations thereof. Optionally, in any of the methods and polymers disclosed herein, each G is independently characterized by formula FX10A, FX10B, FX10C, FX10D, FX10E, or FX10F:

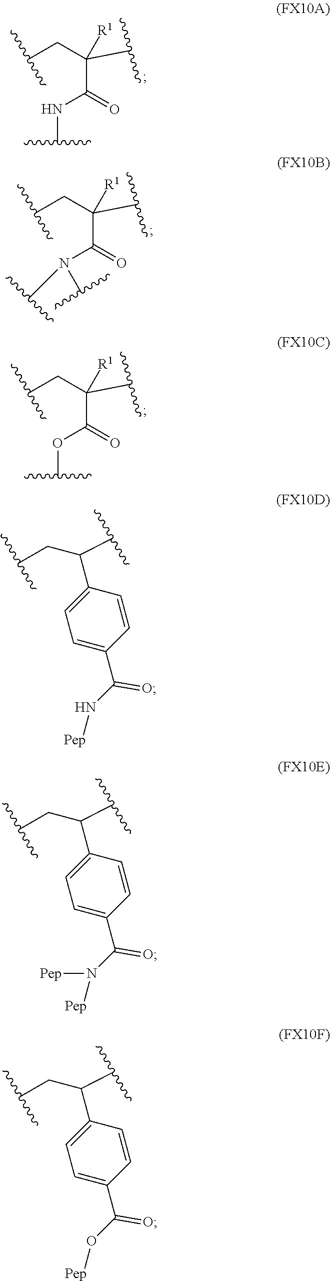

wherein: R¹ is a hydrogen or a methyl group. Optionally, in any of the methods and polymers disclosed herein, each A independently selected from the group consisting of single bond, an oxygen, and one or more substituted or substituted groups having an alkyl group, an alkenylene group, an arylene group, an alkoxy group, an acyl group, a carboxyl group, an aliphatic group, an amide group, an aryl group, an amine group, an ether group, a ketone group, an ester group, a triazole group, a diazole group, a pyrazole group, or combinations thereof. Optionally, in any of the methods and polymers disclosed herein, each A is independently characterized by formula FX3A, FX3B, or FX3C;

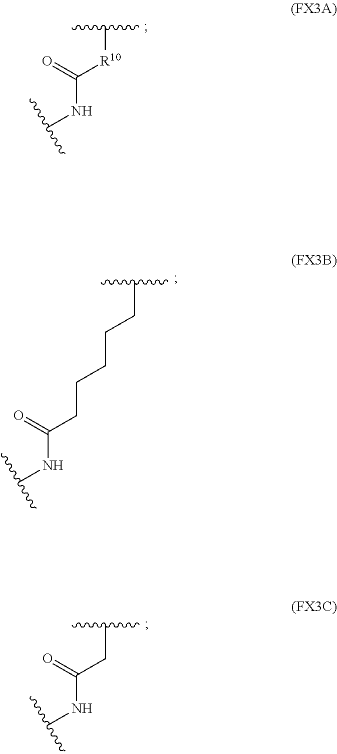

wherein: R¹⁰ is a substituted or unsubstituted C₁-C₁₀ alkyl.

Optionally, in any of the methods and polymers disclosed herein, each peptide moiety comprises a sequence having at least 80% sequence homology with SEQ ID NO:1 (GPLGLAGGWGERDGS), SEQ ID NO:2 (GALTPRGADSGSG), SEQ ID NO: 3 (KLAKLAKKLAKLAK), SEQ ID NO:4 (GSGKEFGADSGSG), SEQ ID NO:5 (GPLGLAGG), SEQ ID NO:6 (HVLVMSATKKKK), SEQ ID NO:7 (GGGCYFQNCPKG) (Terlipressin), SEQ ID NO:8 (DRVYIHPF) (Angiotensin 2), SEQ ID NO: 9 (AQYQDKLAR) (DA1), SEQ ID NO: 10 (GVi(allo)SQIRP) (ABT898), SEQ ID NO:11 (KVPRNQDWL) (gp100), SEQ ID NO:12 (GPLGLAGGWGER), or a combination of these.

Optionally, in any of the methods and polymers disclosed herein, the peptide brush polymer is characterized by formula FX11A or FX11B:

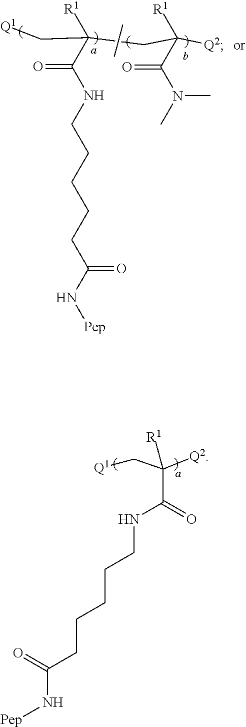

Optionally, in any of the methods and polymers disclosed herein, b is 0 and a is an integer selected from the range of 2 to 100, optionally 10 to 100, optionally 2 to 500, optionally 10 to 500. Preferably, a is an integer selected from the range of 10 to 100. Optionally, in any of the methods and polymers disclosed herein, the peptide brush polymer is characterized by formula FX13B: Q¹-[U¹]ₐ-Q² (FX13B); wherein each of Q¹ and Q² is independently a polymer terminating group; each U¹ is independently a peptide-containing repeating unit; and a is an integer selected from the range of 2 to 500, preferably selected from the range of 2 to 100.

Optionally, in any of the methods and polymers disclosed herein, the peptide brush polymer is the peptide brush polymer is characterized by formula FX12:

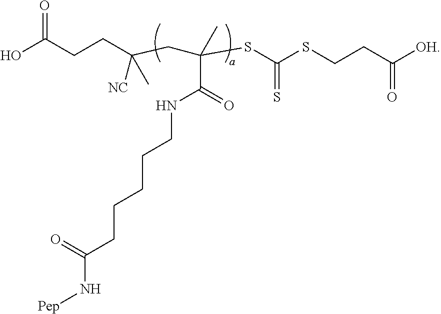

Optionally, in any of the methods and polymers disclosed herein, the peptide brush polymer is the peptide brush polymer is characterized by a degree of polymerization of at least 10 and a peptide graft density of 100%.

Aspects of the invention also include an aqueous solution comprises a peptide brush polymer according to any of the embodiments, or any combination of embodiments, provided herein. Optionally, the peptide brush polymer is in the form of a micelle or nanoparticle in the aqueous solution. Preferably, the aqueous solution is a therapeutic formulation. Preferably, the aqueous solution is a therapeutic formulation acceptable for administering to a human subject to treat or manage a condition in the human subject.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. Synthesis of peptide brush polymers via photo-RDRP. Two bioactive peptide vinyl monomers were designed. A thermolysin-responsive amino acid sequence GPLGLAGG (SEQ ID NO:5), and pro-apoptotic peptide KLAKLAKKLAKLAK (SEQ ID NO: 3).

FIG. 2A. Photo-RDRP of PepAm and DMA in DMSO. FIG. 2B. Kinetic plot of monomer concentrations versus time for PepAm and DMA over the course of photo-RDRP. FIG. 2C. GPC traces of enzyme-responsive peptide brush polymers (P1-P4, Table 1) with different grafting densities. P1-P4 represent poly(PepAm6-co-DMA7), poly(PepAm₁₅-CO-DMA45), poly(PepAm₃₄-co-DMA₁₁₇), and poly(PepAm₂₁-CO-DMA₇₁), respectively. (GPLGLAGG is SEQ ID NO:5.)

FIG. 3A. Schematic of thermolysin-promoted cleavage of poly(PepAm₂₁-CO-DMA₇₁) (P4). FIG. 3B. TEM micrograph of PMMA₉₀-b-poly(PepAm₂₁-CO-DMA₆₃) based micelles (P8) before treatment with thermolysin. FIG. 3C. TEM micrograph of PMMA₉₀-b-poly(PepAm₂₁-CO-DMA₆₃) based micelles (P8) after treatment with thermolysin. FIG. 3D. DLS traces of P8 based nano-objects before and after thermolysin-induced cleavage. (GPLGLAGG is SEQ ID NO:5.)

FIG. 4A. Aqueous photo-RDRP of KLAAm and DMA in acidic buffer (pH 5). FIG. 4B. GPC traces of KLA based peptide brush polymers with different grafting densities (P13-P16, Table 4). FIG. 4C. Circular dichroism spectra of KLA peptide, KLAAm, and representative polymer: poly(KLAAm₂₅-CO-DMA₂₅). (KLAKLAKKLAKLAK is SEQ ID NO:3.)

FIGS. 5A-5D. Flow cytometry analysis (Δ_{ex/em}=548/570 nm) of Hela cells incubated with rhodamine B-labeled KLA peptide (FIG. 5A), poly(KLAAM₂₅-CO-DMA₇₅) (FIG. 5B), poly(KLAAM₂₅-CO-DMA₂₅) (FIG. 5C), and poly(KLAAM₁₀) (FIG. 5D) at a concentration of 0.25 UM with respect to the dye. Chemical structures of each dye-labeled materials are shown adjacent to the corresponding histogram. KLA based peptide brush polymers possessed markedly higher cell uptake ability than that of KLA peptide. (KLAKLAKKLAKLAK is SEQ ID NO:3.)

FIGS. 6A-6L. Confocal laser scanning microscopy images of Hela cells treated with rhodamine-labeled peptide based materials at a concentration of 0.25 UM with respect to rhodamine B (Δ_{ex/em}=548/570 nm). From top to bottom: KLA peptide (FIGS. 6A-6C), poly(KLAAm₂₅-CO-DMA₇₅) (FIGS. 6D-6F), poly(KLAAm₂₅-CO-DMA₂₅) (FIGS. 6G-6I), and poly(KLAAm₁₀) (FIGS. 6J-6L). Cell nuclei were stained with 4′, 6-diamidino-2-phenylindole (DAPI, λ_{ex/em}=360/460 nm). Cell membrane was stained with wheat germ agglutinin, Alexa Fluor 488 conjugate (WGA 488, λ_{ex/em}=495/519 nm). Scale bar: 20 μm, inset scale bar 10 μm.

FIG. 7. Cell viability assay of KLA peptide, KLAAm, and a library of KLA based peptide brush polymers with different grafting densities. Hella cells were treated with peptide based materials and incubated for 72 hours at 37 °C (CellTilter-Blue assay, n=3 independent experiments with three independent samples in each). KLA peptide and KLAAm did not exhibit cytotoxicity to Hela Cells even at a concentration of 100 μM. The IC₅₀ value of peptide brush polymers decreased as the grafting density of peptide brush polymer increased, indicating a higher cytotoxicity of KLA peptide brush polymers with a more compact brush architecture.

FIG. 8. ESI-Mass spectrum of enzyme-responsive peptide monomer.

FIG. 9. HPLC trace of enzyme-responsive peptide monomer. (GPLGLAGG is SEQ ID NO:5.)

FIG. 10. ESI-Mass spectrum of KLA peptide acrylamide monomer.

FIG. 11. HPLC trace of KLA peptide acrylamide monomer.

FIG. 12. ¹H NMR spectrum of KLA peptide acrylamide monomer in d₆-DMSO at 25 °C.

FIG. 13. Assembly of photo-reactor by wrapping the LED strip light inside a beaker.

FIG. 14. ¹H NMR spectra of enzyme-responsive peptide acrylamide (top blue), monomer mixtures before polymerization (middle green), and after polymerization for 18 h (bottom red). (GPLGLAGG is SEQ ID NO:5.)

FIG. 15A. GPC traces of P1 before and after dialysis (cut-off: 20,000 Da). The disappearance of peptide monomer peak clearly indicated that polypeptide was pure after dialysis. FIG. 15B. GPC traces of P1 from both RI detector and UV detector (310 nm).

FIG. 16. Sunlight-induced polymerization of peptide acrylamide in lakeshore at Northwestern University (Evanston campus, Aug. 17, 2018). ¹H NMR based kinetic study of polymerization demonstrated that peptide acrylamide possessed a propagation rate on par with that of the commoner DMA; SEC trace of the final product indicated that the polypeptide product is well-defined with a narrow polydispersity and number-average molecular weight similar to theoretical value (refer to P4 in Table 1). (GPLGLAGG is SEQ ID NO:5.)

FIG. 17. DLS trace of P4 in DIW. The small size (<10 nm) of the polypeptide indicates that the polymer exists as free unimers in DIW. (GPLGLAGG is SEQ ID NO:5.)

FIG. 18. HPLC traces of P1 before and after being treated with thermolysin (1/300 equiv. to the number of peptides in the polymer). No further increase in the peak of the cleaved LAGG fragment was observed after 1 hour, suggesting that the enzyme-induced peptide cleavage was complete within one hour.

FIGS. 19A-19B. ESI-Mass spectra of synthetic LAGG fragment (FIG. 19A) and cleaved LAGG fragment (FIG. 19B) which was collected from HPLC separation. Notably, the cleaved LAGG was from P1 after treatment with thermolysin.

FIG. 20. ¹H NMR spectrum of PMMA₉₀ macroCTA (P5) in CDCl₃ at 25 °C.

FIG. 21. Chain extension of PMMA macroCTA with DMA and PepAm.

FIG. 22. GPC traces of PMMA₉₀ macroCTA (P5), PMMA₉₀-b-poly(PepAm₉-co-DMA₃₀) (P7), and PMMA₉₀-b-poly(PepAm₂₁-CO-DMA₆₃) (P8).

FIG. 23A. TEM image of PMMA₉₀-b-poly(PepAm₉-co-DMA₃₀) in DIW. FIG. 23B. AFM micrographs of PMMA₉₀-b-poly(PepAm₉-co-DMA₃₀) in DIW.

FIG. 24. ¹H NMR spectrum of PMMA₉₀-b-poly(PepAm₉-co-DMA₃₀) (P7), and PMMA₉₀-b-poly(PepAm₂₁-CO-DMA₆₃) (P8) in d₆-DMSO at 25 °C (GPLGLAGG is SEQ ID NO: 5.)

FIG. 25A. Chain extension of PnBA macroCTA with peptide monomers and DMA spacers. FIG. 25B. GPC traces of PnBA macroCTA (P6) and block copolymer PnBA₂₀₀-b-poly(PepAm₃₆-CO-DMA₁₂₃) (P9) indicated a successful chain extension. FIG. 25C. TEM image confirmed the micellar structure of P9 in DIW. (GPLGLAGG is SEQ ID NO: 5.)

FIG. 26. ¹H NMR spectrum of PnBA₂₀₀-b-poly(PepAm₃₆-CO-DMA₁₂₃) (P9) in de-DMSO. (GPLGLAGG is SEQ ID NO:5.)

FIG. 27. Enzyme-promoted shape transformation of PMMA₉₀-b-poly(PepAm₉-co-DMA₃₀) (P7) based micelles. TEM elucidated a transition from spherical micelle to fused worm micelles after treatment with thermolysin. (GPLGLAGG is SEQ ID NO:5.)

FIG. 28. Enzyme-promoted shape transformation of PnBA₂₀₀-b-poly(PepAm₃₆-co-DMA₁₂₃) (P9) based micelles. TEM elucidated a transition from spherical micelle to a mixture of micelles and fiber structure after treatment with thermolysin. (GPLGLAGG is SEQ ID NO:5.)

FIG. 29. DLS traces of PMMA₉₀-b-poly(PepAm₂₁-CO-DMA₆₃) (P7) based nano-objects before and after thermolysin-promoted cleavage. (GPLGLAGG is SEQ ID NO:5.)

FIG. 30. ¹H NMR spectra of reaction mixture (P10) before and after photo-polymerization in DIW. The dramatic reduction in vinyl proton signals (5.4 to 6.8 ppm) indicated a high monomer conversion.

FIG. 31. GPC traces of poly(PepAm-co-DMA) (P10-P12) prepared by aqueous photo-electron transfer RAFT polymerization. Please refer to Table 3 for the information of molecular weights and dispersity of each polymer.

FIG. 32. ¹H NMR spectra of poly(PepAm-co-DMA) (P10-P12) in d₆-DMSO at 25 °C (GPLGLAGG is SEQ ID NO:5.)

FIG. 33. ¹H NMR spectra of reaction mixture (P14) before and after photo-polymerization in DIW. The full diminishment in vinyl proton signals (5.4 to 6.8 ppm) indicated quantitative monomer conversions were achieved for both KLA acrylamide and DMA. (KLA is the peptide KLAKLAKKLAKLAK (SEQ ID NO:3).)

FIG. 34. ¹H NMR spectrum of poly(KLAAm₂₅-co-DMA₂₅) (P14) in d₆-DMSO at 25 °C (KLA is the peptide KLAKLAKKLAKLAK (SEQ ID NO:3).)

FIG. 35. Schematic of the N-acetylation of KLA based polypeptide brushes. (KLA is the peptide KLAKLAKKLAKLAK (SEQ ID NO:3).)

FIG. 36. GPC traces of poly(KLAAm₂₅-co-DMA₁₅₀) (P16) before and after N-acetylation. The protection of amines eliminated the interactions between polymers and the GPC columns, allowing for accurate evaluation of the number-average molecular weights from these amine-abundant polymers. (KLA is the peptide KLAKLAKKLAKLAK (SEQ ID NO:3).)

FIG. 37. ¹H NMR spectra of poly(KLAAm₂₅-co-DMA₁₅₀) (P16) before and after N-acetylation.

FIG. 38. Evaluating cellular uptake ability of KLA based materials using flow cytometry: quantification of rhodamine fluorescence intensity of KLA based materials in Hela cells. The concentration of each material was set to 0.25 UM with respect to Rhodamine B. (data represent mean±s.d., n=3 independent experiments).

FIG. 39. Confocal microscopy images of Hela cells incubated with rhodamine-labeled KLA peptide, poly(KLAAm₁₀), poly(KLAAm₂₅-CO-DMA₂₅), and poly(KLAAm₂₅-CO-DMA₇₅) at a concentration of 0.25 UM with respect to Rhodamine. Cell nuclei were stained with DAPI. Cell membrane was stained with WGA 488. Scale bar: 20 μm, insert scale bar 10 μm.

FIG. 40. ESI-Mass spectrum of rhodamine B labeled KLA peptide (Rho-KLA).

FIG. 41. HPLC trace of rhodamine B labeled KLA peptide.

FIG. 42. Schematic illustration of the one-pot photo-PISA approach to proapoptotic peptide brush polymer nanoparticles.

FIGS. 43A-43H. Synthesis and characterization of KLA peptide brush polymer nanoparticles. FIG. 43A. Synthesis of peptide brush polymer nanoparticles by photo-PISA. FIG. 43B. GPC analysis of peptide brush polymer macroCTA and resulting amphiphilic block copolymers (NP1-NP3). FIG. 43C. DLS traces of peptide brush polymer nanoparticles (NP1-NP3). FIGS. 43D-43H. TEM images of peptide brush polymer nanoparticles (NP1-NP5) with low and high magnifications. (KLAKLAKKLAKLAK is SEQ ID NO:3.)

FIG. 44. Proteolytic cleavage of KLA peptide monomer, KLA brush polymer (poly(KLAAm₁₀-co-DMA₁₀)), and KLA peptide brush polymer nanoparticles (NPs 1-5) in the presence of trypsin (0.1 μM) at 3 7 °C. All the peptide containing materials had a concentration of 200 UM with respect to peptide in PBS buffer (pH=7.4). Data displayed as mean±standard deviation of three independent experiments.

FIG. 45. Cytotoxicity of free KLA peptide, poly [(KLAAm₁₀-CO-DMA₃₀)-b-(DAAm₂₈₀-CO-DMA₁₂₀)] (NP3), and poly [(KLAAm₁₀-CO-DMA₁₀)-b-(DAAm₂₈₀-CO-DMA₁₂₀)] (NP5) using a CellTiter-Blue cell viability assay. Concentrations were calculated with respect to the total KLA peptide content. Hela cells were treated with peptide-containing materials and incubated for 72 h at 37 °C. Data displayed as mean±standard deviation of three independent experiments.

FIG. 46. Assessment of mitochondrial dysfunction induced by the peptide-containing materials using JC-1 probe. Live-cell confocal microscopy images of HeLa cells incubated with KLA peptide, CCCP, NP5 for desired periods of time. Prior to imaging, cells were stained with 2 μM of JC-probe (green, monomer, Δ_{ex/em}=488 nm/510-550 nm; red, J-aggregates, λ_{ex/em}=488 nm/585-649 nm) and then Hoechst 33342 (blue, λ_{ex/em}=405 nm/420-480 nm). Scale bars, 20 μm.

FIG. 47. ESI-MS spectrum of KLAAm.

FIG. 48. HPLC trace of KLAAm.

FIG. 49. ¹H NMR spectrum of KLAAm in DMSO-d6.

FIG. 50. Synthesis of KLA brush polymer based macroCTA. Full monomer conversion was achieved after photo-polymerization for 12 h, as evidenced by disappearance of vinyl protons from 5.5 to 7.0 ppm. (KLAKLAKKLAKLAK is SEQ ID NO: 3.)

FIG. 51. ¹H NMR spectrum of poly(KLAAm₁₀-CO-DMA₃₀) macroCTA in DMSO-d6.

FIG. 52. GPC traces of poly(KLAAm₁₀-co-DMA₃₀) macroCTA monitored with RI and UV detectors.

FIGS. 53A-53C. Evaluation of peptidyl functional group tolerance during the PISA process. Both HPLC and ESI-MS analysis verified that free amines of KLAAm did not react with ketone of DAAm under conditions used in photo-PISA, suggesting the compatibility of KLA peptide with photo-PISA protocol. Notably, the equivalent of reactants (i.e., 30 mg of KLAAm, 21 mg of DAAm, and 340 μL of acidic buffer (pH 5.0)) in this control experiment is identical to the condition used in the preparation of NP1 (Experimental section 3.2 and 3.3).

FIG. 54A. Chain extension of poly(KLAAm₁₀-co-DMA₃₀) macroCTA with DAAm and DMA via photo-PISA. ¹H NMR spectra indicated a full monomer conversion after photo-PISA for 12 h, as evidenced by disappearance of vinyl protons from 5.5 to 7.0 ppm. FIG. 54B. Photographs of NPs 1 and 2 (15 wt. %) prepared via one-pot photo-PISA.

FIG. 55. GPC traces of unimers of P1-P3 and their corresponding core-crosslinked nanoparticles NP1-NP3. The core of NPs was crosslinked with O,O′-1,3-propanediylbishydroxyamine·2HCl (10 mol % with regard to DAAm) prior to the GPC analysis. The condition of crosslinking reaction was adapted according to a previous literature.¹

FIG. 56. Chain extension of poly(KLAAm₁₀-co-DMA₁₀) macroCTA via photo-PISA. GPC traces of block copolymers shifted to shorter elution times after polymerization, indicative of successful chain extension.

FIG. 57. DLS traces of poly [(KLAAm₁₀-co-DMA₁₀)-b-(DAAm₁₄₀-CO-DMA₆₀)] (NP4) and poly [(KLAAm₁₀-CO-DMA₁₀)-b-(DAAm₂₈₀-CO-DMA₁₂₀)] (NP5).

FIG. 58. Representative cryo-TEM images of poly [(KLAAm₁₀-co-DMA₁₀)-b-(DAAm₁₄₀-CO-DMA₆₀)] (NP4) and poly [(KLAAm₁₀-c0-DMA₁₀)-b-(DAAm₂₈₀-CO-DMA₁₂₀)] (NP5). According to images, the size of NP4 (left column) is smaller than that of NP5 (right column). This is in a good agreement with DLS and dry-state TEM results (FIGS. 43A-43H and FIGS. 54A-54B).

FIGS. 59A-59E. Zeta potentials of NPs 1-5 shown in Table 5.

FIG. 60. Circular dichroism spectra of KLA peptide and representative peptide brush polymer nanoparticle (NP1).

FIG. 61. Trypsin-induced cleavage of KLAAm. The initial peptide concentration was 200 UM in PBS. The ratio of trypsin to peptide is 1:2000. The original HPLC peak of KLAAm fully disappeared within 1 h after incubation with trypsin, indicative of 100% cleavage of the peptide.

FIG. 62. Confirmed cleavage mechanism of KLA peptide brush nanoparticles in the presence of trypsin.

FIGS. 63A-63B. Representative trypsin-induced cleavage kinetics of peptide brush polymer nanoparticles (NP1) is elucidated by analytical RP-HPLC and ESI-MS. The HPLC peak area of peptide fragment (LAK, MW=329.44 g/mol) was used to quantify the percentage of cleaved peptides. A standard curve of synthetic LAK, correlating peak area on RP-HPLC chromatograms was used for the determination of the concentration of cleaved LAK after proteolytic cleavage.

FIG. 64. GPC traces of polyDMA₄₀ macroCTA and polyDMA₄₀-b-poly(DAAm₇₀-co-DMA₃₀).

FIG. 65. Dry-state TEM image of polyDMA₄₀-b-poly(DAAm₇₀-co-DMA₃₀) based nanoparticle which was prepared via one-pot photo-PISA at solids content of 15 wt. %. A spherical morphology with an average size of 46 nm was observed.

FIG. 66. Cytotoxicity of polyDMA₄₀-b-poly(DAAmzo-co-DMA₃₀) based nanoparticle using a CellTiter-Blue cell viability assay. Concentrations were calculated with respect to the polymer or CTA content. Hela cells were treated with the materials and incubated for 48 h at 37 °C. A high cell viability of Hela cells was observed for this peptide-free polymer nanoparticles even at a high polymer concentration at 20 μM. Data displayed as mean±standard deviation of three independent experiments.

FIGS. 67A-67D. Dry-state TEM images and DLS traces of rhodamine B labeled nanoparticles (i.e., Rho-NP3 and Rho-NP5).

FIG. 68. Flow cytometry analysis (λ_{ex/em}=548/570 nm) of Hela cells incubated with Rho-KLA peptide, Rho-NP3, and Rho-NP5 at a concentration of 0.25 UM with respect to Rhodamine.

FIG. 69. Confocal microscopy images of Hela cells incubated with rhodamine-labeled KLA peptide, NP3, and NP5 at a concentration of 0.25 UM with respect to Rhodamine. Cell nuclei were stained with DAPI. Cell membrane was stained with WGA 488. Scale bar: 20 μm.

FIG. 70. Summary and formulas of certain embodiments of methods, reagents, and repeating units of polymers disclosed herein.( ) (GPLGLAGGWGERDGS is SEQ ID NO:1; GALTPRGADSGSG is SEQ ID NO:2; KLAKLAKKLAKLAK is SEQ ID NO: 3; GSGKEFGADSGSG is SEQ ID NO:4.)

FIG. 71. ESI-MS spectrum of MAm-GALTPRGADSGSG (GALTPRGADSGSG is SEQ ID NO:2).

FIG. 72. HPLC trace of MAm-GALTPRGADSGSG (SEQ ID NO:2).

FIG. 73. ¹H NMR spectrum of MAm-GALTPRGADSGSG (SEQ ID NO:2).

FIG. 74. ¹H NMR spectrum of poly(MAm-GALTPRGADSGSG) (SEQ ID NO:2) in DMSO-d6.

FIG. 75. GPC trace of poly(MAm-GALTPRGADSGSG) (SEQ ID NO:2) in DMF eluent.

FIG. 76. ESI-MS spectrum of MAm-GPLGLAGGWGERDGS (GPLGLAGGWGERDGS is SEQ ID NO:1).

FIG. 77. HPLC trace of MAm-GPLGLAGGWGERDGS (SEQ ID NO:1).

FIG. 78. ¹H NMR spectrum of poly(MAm-GPLGLAGGWGERDGS) (SEQ ID NO: 1) in DMSO-d6.

FIG. 79. GPC trace of poly(MAm-GPLGLAGGWGERDGS) (SEQ ID NO:1) in DMF eluent.

FIG. 80. DLS trace of poly(MAm-GPLGLAGGWGERDGS) (SEQ ID NO:1) in DPBS buffer (pH=7.4).

FIG. 81. ESI-MS spectrum of MAm-KLAKLAKKLAKLAK (KLAKLAKKLAKLAK is SEQ ID NO:3).

FIG. 82. HPLC trace of MAm-KLAKLAKKLAKLAK (SEQ ID NO:3).

FIG. 83. ¹H NMR spectrum of MAm-KLAKLAKKLAKLAK (SEQ ID NO:3).

FIG. 84. ¹H NMR spectrum of poly(MAm-KLAKLAKKLAKLAK) (SEQ ID NO:3) in DMSO-d6.

FIG. 85. GPC trace of poly(MAm-KLAKLAKKLAKLAK) (SEQ ID NO:3) in DMF eluent.

FIGS. 86A-86C. Formulas of exemplary peptide brush polymers according to embodiments disclosed herein. Block copolymers such as that of FIG. 86C, according to embodiments described herein, can form nanoparticles.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and a high number average molecular weight (e.g. greater than or equal to 100 Da, in some embodiments greater than or equal to 10 kDa, in some embodiments greater than or equal to 50 kDa, in some embodiments greater than or equal to 100 kDa). In some embodiments, polymers are commonly the polymerization product of one or more monomer precursors (i.e., polymerizable monomers). Copolymers may comprise two or more different types or compositions of monomer units, and include random, block, brush, brush block, alternating, segmented, grafted, tapered and other architectures. Useful polymers include organic polymers that may be in amorphous, semi-amorphous, crystalline or semi-crystalline states. Cross linked polymers having linked monomer chains are useful for some applications, for example linked by one or more disulfide linkages. The invention includes polymers comprising (poly) peptide side chain. Optionally, the peptide side chains are therapeutic agents or comprise therapeutic agent(s). Preferably, in any of the methods and polymers disclosed herein, each of at least 30%, preferably at least 50%, preferably at least 70%, more preferably at least 80%, further more preferably at least 90%, further more preferably all of the peptide moieties is a therapeutic peptide.

The term polymer includes homopolymers. As used herein, the term “homopolymer” preferably refers to a brush polymer having a 100% peptide density wherein each monomer unit or repeating unit of said brush polymer comprises a side chain group with a peptide moiety, or in other words, each repeating unit of said homopolymer is a peptide-containing repeating unit. As used herein, each peptide sequence of a homopolymer is not necessarily identical, such that a homopolymer optionally includes more than one peptide sequence. Optionally, each peptide sequence of a homopolymer is identical. In some embodiments, the term “homopolymer” is used to describe a single polymer block of a block polymer, in which case each repeating unit of said single polymer block is a peptide-containing repeating unit.

An “oligomer” refers to a molecule composed of repeating structural units connected by covalent chemical bonds often characterized by a number of repeating units less than that of a polymer (e.g., equal to or less than 3 repeating units) and a lower molecular weights (e.g. less than or equal to 1,000 Da) than polymers. Oligomers may be the polymerization product of one or more polymerizable monomers (also referred to as monomer precursors).

A “polypeptide” or “oligopeptide” herein are used interchangeably and refer to a polymer of repeating structural units connected by a peptide bond. Typically, the repeating structural units of the polypeptide are amino acids including naturally occurring amino acids, non-naturally occurring amino acids, analogues of amino acids or any combination of these. The number of repeating structural units of a polypeptide, as understood in the art, are typically less than a “protein”, and thus the polypeptide often has a lower molecular weight than a protein. Peptides and peptide moieties, as used and described herein, comprise two or more amino acid groups connected via peptide bonds.

Amino acids and amino acid groups refer to naturally-occurring amino acids, unnatural (non-naturally occurring) amino acids, and/or combinations of these. Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to herein by either the commonly known three letter symbols or by the one-letter symbols recommended by the IUPAC-IUB Biochemical Nomenclature Commission.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e. adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g. [A][B]), or may be provided in a selected sequence ([A][B][A][B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Polymer backbone group” refers to groups that are covalently linked to make up a backbone of a polymer, such as a block copolymer. Polymer backbone groups may be linked to side chain groups, such as polymer side chain groups. Some polymer backbone groups useful in the present compositions are derived from polymerization of a monomer selected from the group consisting of a substituted or unsubstituted, olefin, vinyl, acrylate, acrylamide, cyclic olefin, norbornene, norbornene anhydride, cyclooctene, cyclopentadiene, styrene and acrylate. Some polymer backbone groups useful in the present compositions are obtained from metal-free photoinduced reversible-deactivation radical polymerization (photo-RDRP), photo-electron transfer reversible addition-fragmentation transfer polymerization (PET-RAFT), and/or photoinitiated polymerization-induced self-assembly (photo-PISA). Polymer backbones may terminate (e.g., by coupling, disproportionation, or chain transfer) in a range of backbone terminating groups including, but not limited to, hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylate, catechol, or any combinations thereof; wherein each of R³⁰-R⁴² is independently hydrogen, C₁-C₁₀ alkyl or C₅-C₁₀ aryl. A polymer backbone may terminate in backbone terminating groups that is a portion or moiety from a chain transfer used during polymerization of the polymer.

As used herein, the term “chain transfer agent” refers to a compound that reacts with a growing polymer chain to interrupt growth and transfer the reactive species to a different compound (e.g., different polymer chain, monomer, or polymerizable monomer). The chain transfer agent can help regulate the average molecular weight of a polymer by terminating polymerization. Exemplary chain transfer agents include, but are not limited to, compounds with one or more trithiocarbonate or dithioester groups, compounds with one or more carboxylic acid groups, and compounds with a combination of these. Useful chain transfer agents for polymerization as used herein include, for example, 4-((((2-carboxyethyl)thio) carbonothioyl)thio)-4-cyanopentanoic acid (CAS Number 2055041-03-5), 4-Cyano-4-(((ethylthio) carbonothioyl)thio) pentanoic acid (CAS Number 1137725-46-2), 4-Cyano-4-(phenylcarbonothioylthio) pentanoic acid (CAS Number 201611-92-9), derivatives thereof, and substituted variations thereof. Useful chain transfer agents for polymerization as used herein include, for example, chain transfer agents with a dithioester group.

“Polymer side chain group” refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition. A polymer side chain group may be directly or indirectly linked to the polymer backbone groups. In some embodiments, polymer side chain groups provide steric bulk and/or interactions that result in an extended polymer backbone and/or a rigid polymer backbone. Some polymer side chain groups useful in the present compositions include unsubstituted or substituted polypeptide groups. Some polymers useful in the present compositions comprise repeating units obtained via anionic polymerization, cationic polymerization, free radical polymerization, group transfer polymerization, a photopolymerization, a ring-opening polymerization, metal-free photoinduced reversible-deactivation radical polymerization (photo-RDRP), photo-electron transfer reversible addition-fragmentation transfer polymerization (PET-RAFT), and/or photoinitiated polymerization-induced self-assembly (photo-PISA). A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane acrylate, or catechol; wherein each of R³⁰_R⁴² is independently hydrogen or C₁-C₅ alkyl.

As used herein, the term “brush polymer” refers to a polymer comprising repeating units each independently comprising a polymer backbone group directly or indirectly covalently linked to at least one polymer side chain group. A brush polymer may be characterized by brush density, which refers to the percentage of the repeating units in a brush polymer that comprise a polymer side chain group. Brush polymers of certain aspects are characterized by a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a density selected from the range of 75% to 100%, or optionally for some embodiments a density selected from the range of 90% to 100%.

The terms “monomer” or “polymerizable monomer” can be used interchangeably and refer to a monomer precursor capable of undergoing polymerization as described herein to form a polymer according to embodiments described herein. The term “polymerizable monomer” is also interchangeably referred to herein as a “monomer precursor.” Generally, the “monomer” or “polymerizable monomer” comprises an olefin capable of undergoing polymerization as described herein.

The terms “monomer unit,” “repeating monomer unit,” “repeating unit,” and “polymerized monomer” can be used interchangeably and refer to a monomeric portion of a polymer described herein which is derived from or is a product of polymerization of one individual “monomer” or “polymerizable monomer.” Each individual monomer unit of a polymer is derived from or is a product of polymerization of one polymerizable monomer. Each individual “monomer unit” or “repeating unit” of a polymer comprises one (polymerized) polymer backbone group. For example, in a polymer that comprises monomer units X and Y arranged as X-Y-X-Y-X-Y-X-Y (where each X is identical to each other X and each Y is identical to each other Y), each X and each Y is independently can be referred to as a repeating unit or monomer unit.

As used herein, the term “degree of polymerization” refers to the average number of monomer units per polymer chain. Since the degree of polymerization can vary from polymer to polymer, the degree of polymerization is generally represented by an average which can be determined by, for example, gel permeation chromatography with a multi-angle light scattering detector (GPC-MALS). The degree of polymerization can be calculated by the number-average molecular weight of polymer (e.g., determined by GPC-MALS) dividing by the molar mass of the monomer.

As used herein, the terms “peptide density” and “peptide graft density” interchangeably refer to the percentage of monomer units in the polymer chain which have a peptide covalently linked thereto. The percentage is based on the overall sum of monomer units in the polymer chain. For example, for certain polymers described herein, each P¹ is the polymer side chain comprising the peptide, each P² is a polymer side chain having a composition different from that of P¹, and each S is independently a repeating unit having a composition different from P¹ and P². Thus, the peptide density of P¹, or percentage of monomer units comprising the peptide of P¹ (i.e., P¹ for this particular example) would be represented by the formula:

$$\frac{{P}^{1}}{{P}^{1} + {P}^{2} + S} \times 100 ,$$

where each variable refers to the number of monomer units of that type in the polymer chain.

In an aspect, the polymer side chain groups can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6±5 angstroms apart on the polymer backbone. In some embodiments the brush polymer has a high a brush density (e.g. greater than 70%), wherein the polymer side chain groups are spaced 5 to 20 angstroms apart on the polymer backbone.

The term “sequence homology” or “sequence identity” means the proportion of amino acid matches between two amino acid sequences. When sequence homology is expressed as a percentage, e.g., 50%, the percentage denotes the fraction of matches over the length of sequence that is compared to some other sequence. Gaps (in either of the two sequences) are permitted to maximize matching; for example, wherein gap lengths of 5 amino acids or less, optionally 3 amino acids or less, are usually used.

The term “fragment” refers to a portion, but not all of, a composition or material, such as a polypeptide composition or material. In an embodiment, a fragment of a polypeptide refers to 50% or more of the sequence of amino acids, optionally 70% or more of the sequence of amino acids and optionally 90% or more of the sequence of amino acids.

“Polymer blend” refers to a mixture comprising at least one polymer, such as a brush polymer, e.g., brush block copolymer, and at least one additional component, and optionally more than one additional component. In some embodiments, for example, a polymer blend of the invention comprises a first brush copolymer and one or more addition brush polymers having a composition different than the first brush copolymer.

In some embodiments, for example, a polymer blend of the invention further comprises one or more additional brush block copolymers, homopolymers, copolymers, block copolymers, brush block copolymers, oligomers, solvent, small molecules (e.g., molecular weight less than 500 Da, optionally less than 100 Da), or any combination of these. Polymer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

A “peptide-containing monomer” is a monomer species that comprises a peptide moiety. Preferably, a peptide-containing monomer comprises a polymer backbone precursor group, one or more covalent anchor (or, linker) groups covalently attached to the backbone precursor group, and at least one peptide moiety (generally, one or two peptide moieties) each independently covalently attached to an anchor group. The term “polymer backbone precursor group” refers to a polymerizable group of a monomer that forms a polymer backbone group upon polymerization of said monomer.

A “peptide-containing polymer block” is a polymer block that comprises at least one peptide moiety. A peptide-containing polymer block comprises peptide-containing repeating units. A “peptide-containing repeating unit” is a repeating unit of the polymer block that comprises a peptide moiety. Preferably, a peptide-containing repeating unit comprises a polymer backbone group, one or more covalent anchor (or, linker) groups covalently attached to the polymer backbone group, and at least one peptide moiety (generally, one or two peptide moieties) each independently covalently attached to an anchor group. The polymer backbone group of each repeating unit, such as of a peptide-containing repeating unit or a peptide-free repeating unit, is directly or indirectly covalently attached to a polymer backbone group of at least one other repeating unit.

The term “peptide-free polymer block” refers to a polymer block that is free of peptide moieties. A peptide-free polymer block can comprise side chain group that are free of a peptide moiety. A peptide-free-polymer block has peptide-free repeating units. A “peptide-free repeating unit” is a repeating unit of the polymer block that does not have a peptide moiety.

A “peptide brush polymer” is a brush polymer comprising polymer side chain groups each comprising one or more peptide moieties.

A “peptide moiety” is a moiety or group that comprises or consists of a peptide.

The term “monomer conversion” refers to a fraction, typically expressed as a percentage, of monomers provided for polymerization and exposed to polymerization conditions that are polymerized to form a polymer by creating monomer units. Monomer conversion can be calculated by nuclear magnetic resonance spectroscopy which determines the remaining fraction of vinyl protons (5.5-6.5 ppm) after the polymerization, wherein:

Monomer conversion=[initial intensity of vinyl protons−remaining intensity of vinyl protons]/[initial intensity of vinyl protons]

As used herein, the term “photoinitiator” refers to a compound that creates a reactive species (e.g., free radicals, cations or anions) when exposed to light (such as visible and/or ultraviolet light). A photoinitiator is optionally a photocatalyst, and vice versa. Exemplary photoinitiators include but are not limited to eosin Y disodium, pentamethyldiethylenetriamine, sodium phenyl-2,4,6-trimethylbenzoylphosphinate, lithium phenyl-2,4,6-trimethylbenzoylphosphinate, Zn(II) meso-Tetra(4-sulfonatophenyl) porphine (optionally together with ascorbic acid), substituted variations of any of these, derivatives of any of these, and combinations of these. For example, Shanmugam, et al. (S. Shanmugam, 2016, “Aqueous RAFT Photopolymerization with Oxygen Tolerance,” Macromolecules 2016, 49, 24, 9345-9357, doi: 10.1021/acs.macromol.6b02060), which is incorporated herein by reference, uses Zn(II) meso-Tetra(4-sulfonatophenyl) porphine with ascorbic acid for RAFT photopolymerization.

The term “photopolymerization” refers to a polymerization process that uses light (such as visible and/or ultraviolet light) to initiate and propagate a polymerization reaction to form a polymer. Preferably, but not necessarily, photopolymerization described herein can be initiated by visible light.

As used herein, visible light refers to any suitable electromagnetic radiation the wavelength(s) of which are about 380 nm to about 740 nm. A particularly preferable range of wavelengths of visible light suitable for photopolymerization is 380 nm to 500 nm. Another particularly preferable range of wavelengths of light suitable for photopolymerization is 500 nm to 700 nm.

As used herein, ultraviolet light refers to any suitable electromagnetic radiation the wavelength(s) of which are about 10 nm to about 380 nm. A particularly preferable range of wavelengths of ultraviolet light suitable for photopolymerization is 300 nm to 380 nm, preferably 320 nm to 380 nm.

When referring to a material, such as a polymer, being aqueous, the term “aqueous” refers to said material being dispersed, dissolved, or otherwise solvated by water. Preferably, an aqueous material, such as an aqueous peptide brush polymer, is water-soluble. An “aqueous solution” refers to a solution that comprises water as solvent and one or more solute species dispersed, dissolved, or otherwise solvated by the water. An aqueous process, such as a polymerization, is a process taking place in an aqueous solution. Optionally, but not necessarily, an aqueous solution or an aqueous solvent includes 20 vol. % or less, optionally 15 vol. % or less, optionally 10 vol. % or less, preferably 5 vol. % or less, of a non-aqueous or organic solvent.

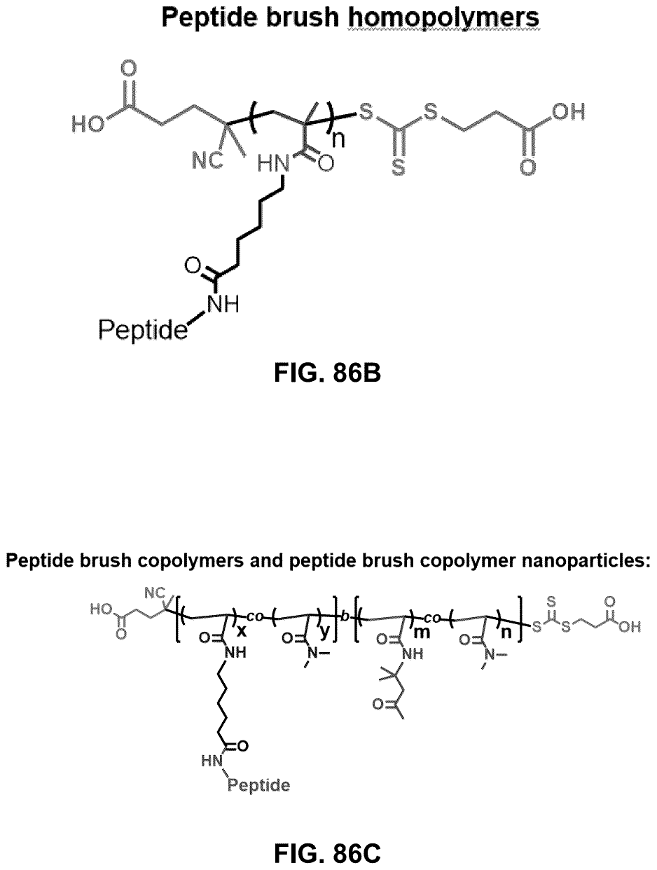

As used herein, the term “-co-” as used in a formula, such as in “poly [(KLAAm₁₀-co-DMA₃₀)-b-(DAAm280-co-DMA₁₂₀)]” and in FIG. 86A, indicates that the monomer or repeating units on either side of “-co-” together form a copolymer or have been copolymerized. The repeating units separated by “-co-” can be covalently linked randomly or in any order. As used herein, the term “-b-” as used in a formula, such as in “poly [(KLAAm₁₀-co-DMA₃₀)-b-(DAAm280-co-DMA₁₂₀)]” and in FIG. 86C, indicates that the repeating units or copolymers separated by “-b-” are covalently attached blocks that have been copolymerized to form a block copolymer. The units or blocks separated by “-b-” can be covalently linked randomly or in any order.

The term “metal-free” refers to a chemical species, such as a monomer, a polymer, a chain transfer agent, a photoinitiator, or another molecule or compound, whose chemical formula is free of a metal element. Preferably, a metal-free chemical species provided to a mixture, reaction, or method described herein, comprises less than 5%, preferably less than 1%, preferably less than 0.1%, more preferably less than 0.01%, more preferably less than 0.001%, further more preferably less than 0.0001% by mass of metal or metal-containing impurities. Preferably, a metal-free chemical species provided to a mixture, reaction, or method described herein, is free of a metal or metal-containing impurities. Preferably, all chemical species, including any catalysts, used in polymerization reactions according to methods disclosed herein are metal-free chemical species. The terms metal and metal element are exclusive of B, Si, and Se.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

Unless otherwise specified, the term “average molecular weight,” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As is customary and well known in the art, hydrogen atoms in formulas presented throughout herein, such as, but not limited to formulas FX2A, FX2B, FX2C, FX2D, FX2E, FX2F, FX3A, FX3B, FX5A, FX5B, FX5C, FX10A, FX10B, FX10C, FX10D, FX10E, FX10F, FX11A, FX11B, and FX12, are not always explicitly shown, for example, hydrogen atoms bonded to the carbon atoms of aromatic, heteroaromatic, and alicyclic rings are not always explicitly shown in formulas presented herein. The structures provided herein, for example in the context of the description of formulas just listed and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g. L¹-L²).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g. L¹-L²).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g. L¹-L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g. L¹-L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g. L¹-L²).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₅ cycloalkenylene groups, for example, as one or more linking groups (e.g. L¹-L²).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g. L¹-L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy) n-alkoxy wherein n is an integer from 1 to 10, preferably 1 to 4, and more preferably for some embodiments 1 to 3.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As used herein, the term “substituted” can mean that one or more hydrogens on the designated atom or group (e.g., substituted alkyl group) are replaced with another group provided that the designated atom's normal valence is not exceeded. For example, when the substituent is oxo (i.e., ═O), then two hydrogens on the atom are replaced. The substituent group can be any substituent group described herein. For example, substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including-CN;
—COOR where R is a hydrogen or an alkyl group or an aryl group and more specifically where R is a methyl, ethyl, propyl, butyl, or phenyl group all of which groups are optionally substituted;
—COR where R is a hydrogen or an alkyl group or an aryl group and more specifically where R is a methyl, ethyl, propyl, butyl, or phenyl group all of which groups are optionally substituted;
—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group and more specifically where R is hydrogen, methyl, ethyl, propyl, butyl, or a phenyl group, which are optionally substituted;
—SO₂R, or —SOR where R is an alkyl group or an aryl group and more specifically where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;
—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g. achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g. transcription factor) relative to the absence of the antagonist. An “activity increasing amount,” as used herein, refers to an amount of agonist (activator) required to increase the activity of an enzyme or protein (e.g. transcription factor) relative to the absence of the agonist. A “function disrupting amount,” as used herein, refers to the amount of antagonist (inhibitor) required to disrupt the function of an enzyme or protein (e.g. transcription factor) relative to the absence of the antagonist. A “function increasing amount,” as used herein, refers to the amount of agonist (activator) required to increase the function of an enzyme or protein (e.g. transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein

The term “modulator” refers to a composition that increases or decreases the level of a target molecule or the function of a target molecule.

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

The term “non-peptide therapeutic moiety” refers to a therapeutic moiety that is not a peptide or a polypeptide having at least 2 amino acids. A therapeutic moiety refers to a therapeutic agent covalently attached to compound or molecule, such as a polymer according to any of the embodiments disclosed herein. A therapeutic moiety is optionally a monovalent moiety. The therapeutic moiety is a therapeutic agent that is a therapeutically or pharmaceutically active therapeutic agent when attached to the polymer, when released from the polymer (such as via a chemical reaction), or both. A therapeutic agent is capable of treating or managing a condition, such as a disease, in a living subject, such as a human or animal. A non-peptide therapeutic moiety is optionally a small molecule having a molecular weight below 4500 Da, optionally below 2000 Da, optionally below 1000 Da. Unless otherwise stated, a peptide or polypeptide of the invention can be a therapeutic peptide, which is a therapeutic moiety that is or that comprises a peptide or polypeptide. Optionally the term “peptide” can refer to a polypeptide.

Optionally in any of the polymers, methods, and compositions disclosed herein, at least a fraction of all side chain moieties in the polymer that are side chain moieties comprising a peptide moiety are selected to provide for cellular uptake. The term “cellular uptake” refers to any process or mechanism that results in a molecule, peptide, therapeutic agent, compound, polymer, or portion thereof, or material being transported either actively of passively across the cellular membrane of a biological cell. Optionally, cellular uptake refers to cellular uptake of or penetration of a biological by at least a portion of the polymer, the majority of the polymer, or the entirety of the polymer. Cellular uptake can be measured or quantified, such as via absorbance or fluorescence signal unique to a portion of the polymer (such as the drug) using different cellular assays, UV-Vis absorption spectroscopy, fluorescence spectroscopy, radio labeling, mass-spectroscopy, and/or inductively coupled plasma mass spectrometry. Optionally in any of the polymers, methods, and liquid compositions disclosed herein, at least one of the plurality of peptide moieties is a non-cell-penetrating peptide. Optionally in any of the polymers, methods, and liquid compositions disclosed herein, each peptide moiety of at least a majority of the plurality of peptide moieties is a non-cell-penetrating peptide. Optionally in any of the polymers, methods, and liquid compositions disclosed herein, the polymer has a net positive charge. Preferably, the net positive charge of the polymer is present at least when the polymer is exposed to physiological conditions, including normal physiological conditions. Preferably, any positive charge of the polymer is present at least when the polymer is exposed to physiological conditions, including normal physiological conditions. Preferably in any of the polymers, methods, and liquid compositions disclosed herein, at least one of the plurality of peptide moieties has a positive charge. The presence of a positive charge can increase or otherwise enhance the therapeutic activity or function of the polymer, or portions thereof such as of the non-peptide therapeutic(s) and any therapeutic peptides, if present. In embodiments, the presence of a positive charge on the polymer can increase or otherwise enhance the therapeutic activity or function of the polymer, or portions thereof at least because of the enhanced or improved cellular uptake efficiency of the polymer due to the presence of the positive charge. Preferably, polymers disclosed herein can penetrate or be taken up by a biological cell even when any, a majority, or even when all of the peptide sequences on said polymer do not correspond to cell-penetrating peptides. This is because peptide sequences that are not cell-penetrating peptides but that have at least a single positive charge are able to enter cells (cellular uptake) once polymerized as a high density brush of peptides, wherein, in contrast, the monomeric peptide alone would be unable to enter the cell. See also Blum, et al. (“Activating peptides for cellular uptake via polymerization into high density brushes.” A. P. Blum, J. K. Kammeyer and N. C. Gianneschi, Chem. Sci., 2016, 7, 989-994), which is incorporated herein by reference in its entirety to the extent not inconsistent herewith.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value. The term “substantially” refers to a property, condition, or value that is within 20%, 10%, within 5%, within 1%, optionally within 0.1%, or is equivalent to a reference property, condition, or value. The term “substantially equal”, “substantially equivalent”, or “substantially unchanged”, when used in conjunction with a reference value describing a property or condition, refers to a value that is within 20%, within 10%, optionally within 5%, optionally within 1%, optionally within 0.1%, or optionally is equivalent to the provided reference value. The term “substantially greater”, when used in conjunction with a reference value describing a property or condition, refers to a value that is at least 1%, optionally at least 5%, optionally at least 10%, or optionally at least 20% greater than the provided reference value. The term “substantially less”, when used in conjunction with a reference value describing a property or condition, refers to a value that is at least 1%, optionally at least 5%, optionally at least 10%, or optionally at least 20% less than the provided reference value.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

The invention can be further understood by the following non-limiting examples.

### Example 1A: Bioactive Peptide Brush Polymers Via Photoinduced Reversible-Deactivation Radical Polymerization

Abstract: Harnessing metal-free photoinduced reversible-deactivation radical polymerization (photo-RDRP) in organic and aqueous phases, we report a synthetic approach to enzyme-responsive and pro-apoptotic peptide brush polymers. Thermolysin-responsive peptide based polymeric amphiphiles assembled into spherical micellar nanoparticles that undergo a morphology transition to worm-like micelles upon enzyme-triggered cleavage of coronal peptide sidechains. Moreover, pro-apoptotic polypeptide brushes show enhanced cell uptake over individual peptide chains of the same sequence, resulting in a significant increase in cytotoxicity to cancer cells. Importantly, increased grafting density of pro-apoptotic peptides on brush polymers correlates with increased uptake efficiency and concurrently, cytotoxicity. The mild synthetic conditions afforded by photo-RDRP, make it possible to access well-defined peptide-based polymer bioconjugate structures with tunable bioactivity.

Introduction: The convergence of photochemistry and controlled polymerization techniques has led to the development of new living polymerization methodologies, post-polymerization modification strategies, and to the production of advanced materials.^{[1]} In comparison with common triggers for polymerization, light has the unique advantage of providing mild reaction conditions, without the need for adding additional reactive molecules, and providing spatiotemporal control over reactions.^{[2-6]} The toolbox of photo-induced controlled polymerization techniques is expanding, giving rise to photo-induced reversible deactivation radical polymerization (photo-RDRP),^{[1-6]} photo-induced ring-opening metathesis polymerization (photo-ROMP),^{[7]} photo-controlled cationic polymerization,^{[8]} and photo-triggered ring opening polymerization (photo-ROP) of cyclic esters or N-carboxyanhydride.^{[9]} Among these photo-induced polymerization methods, photo-RDRP techniques have received the most interest due to their broad vinyl monomer scope and relatively mild reaction conditions conducted at room temperature, in metal-free systems, and with high tolerance to oxygen and water.^{[10-11]} We reasoned that these mild conditions should provide a route for the incorporation of peptide-modified vinyl monomers into bioactive, highly functionalized polymers, and polymeric materials. The mild conditions would minimize side reactions and thus retain the integrity of the biomolecules during polymerization and provide clean materials following polymerization.^{[6, 12-13]}

Photo-electron transfer reversible addition-fragmentation transfer polymerization (PET-RAFT) represents a powerful tool in the arsenal of photo-RDRP approaches.^{[5]} This technique can be performed under visible blue or green light in the presence of a biocompatible organo-photocatalyst such as eosin Y.^{[2]} More generally, RAFT polymerization has demonstrated tolerance towards many functional groups pendent on monomers. Therefore, we postulated that PET-RAFT could serve as an ideal photo-RDRP approach to explore photo polymerization of peptide-modified vinyl monomers.

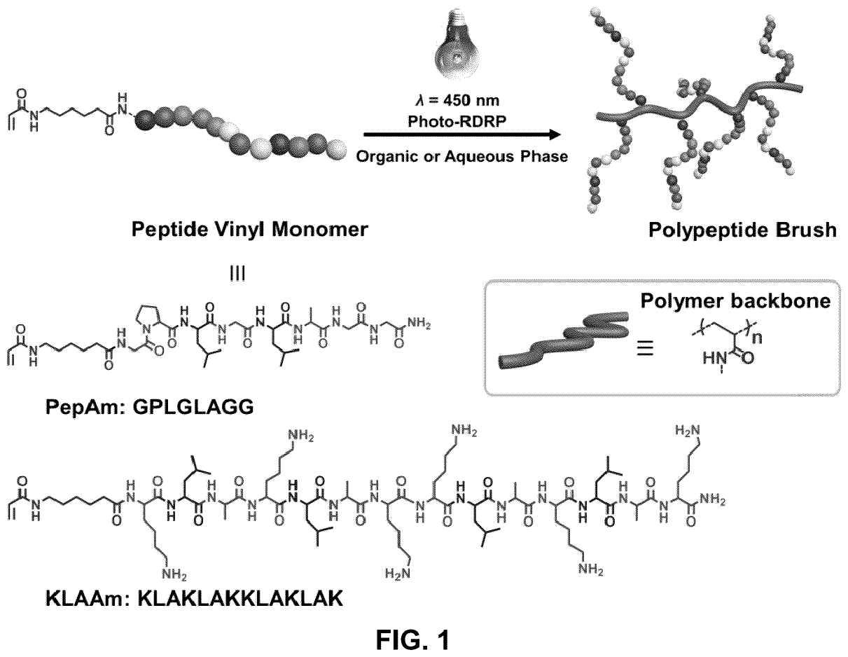

The multi-valent display of peptides as side chains in brush polymers can lead to materials with enhanced biological activities such as higher binding affinities to targets and increased cell-penetration.^{[15-17]} Examples include peptide brush polymers prepared via ring-opening metathesis polymerization (ROMP) and atom transfer radical polymerization which involve the use of ruthenium and copper-based catalysts.^{[18-20]} The possibility of residual heavy metals remaining after synthesis raises concerns in biomedical applications. Herein we demonstrate a metal-free photo-RDRP approach to peptide brush polymers (FIG. 1). Two bioacitive peptide vinyl monomers featuring enzyme-responsive and pro-apoptotic amino acid sequences were successfully copolymerized with dimethylacrylamide (DMA) via PET-RAFT protocol in both organic and aqueous solutions. Trithiocarbonate based RAFT agents were used because the resultant polymers with terminal trithiocarbonate moeity have been demonstrated non-toxic in vitro and can be easily removed upon the polymerization. Incorporation of the DMA comonomer not only lessened the steric hindrance from the peptide macromonomer, but also facilitated the preparation of peptide brush polymers with different grafting densities. Furthermore, the robust nature of PET-RAFT allowed access to various architectures including brush and linear-brush diblock copolymers consisting of enzyme-responsive peptide side chains. Linear-brush diblock copolymers self-assembled into micelles, capable of further morphing into worm-like structure upon treatment with thermolysin. By variation of the grafting density of pro-apoptotic peptide, the cellular uptake efficiency and cytotoxicity of peptide brush polymers can be controlled, revealing the crucial role of architecture (i.e., grafting density) in governing the bioactivity of polypeptide brushes. These results highlight the potential of photo-RDRP for the preparation of peptide brush polymer materials with well-defined structures and highly tunable properties in biomedical applications.

Results and Discussion: Peptide monomers containing acrylamide moieties, serving as the polymerizable group for radical polymerization were synthesized by addition of acrylic acid to an amino-hexanoic spacer unit on the N-terminus of the peptide chain (FIG. 1).

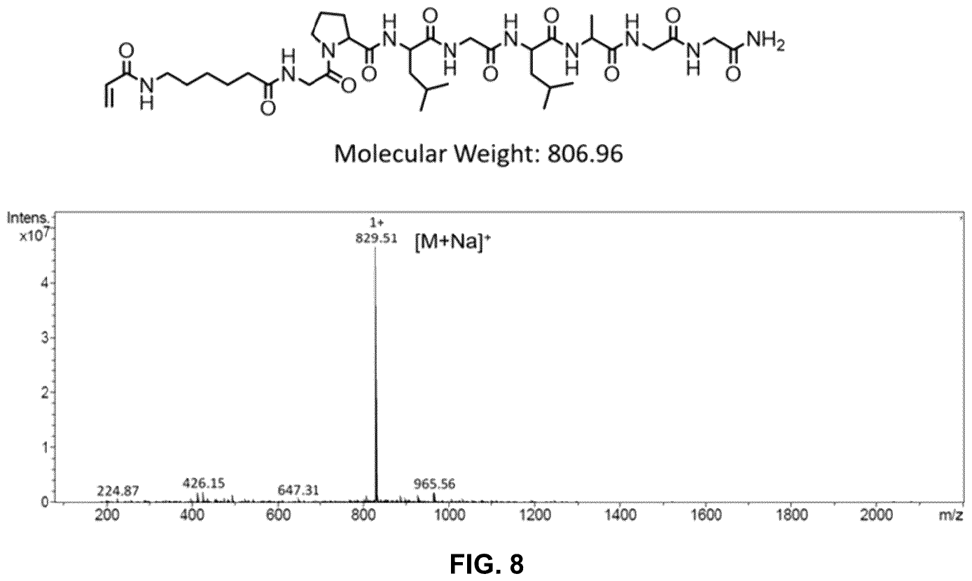

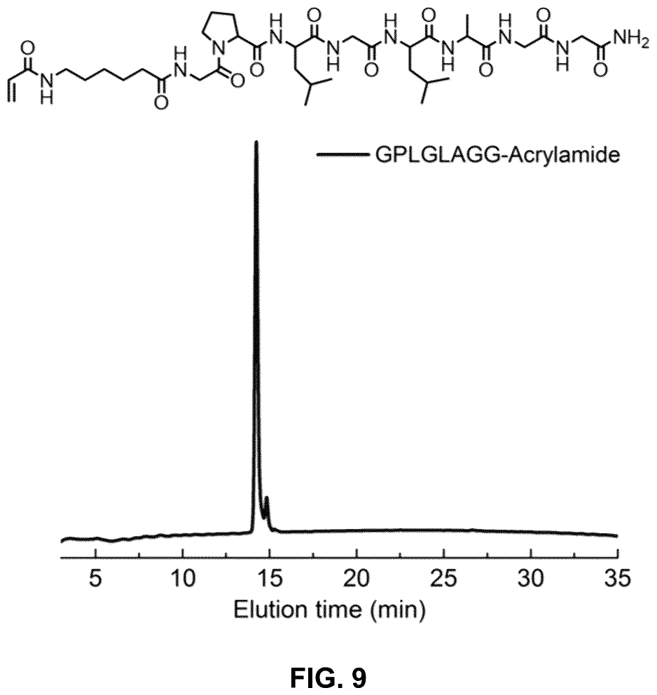

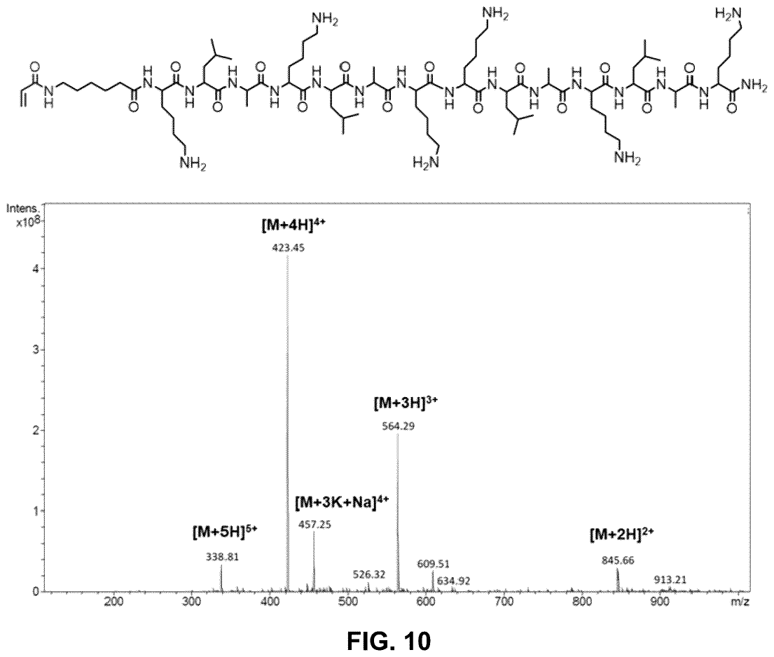

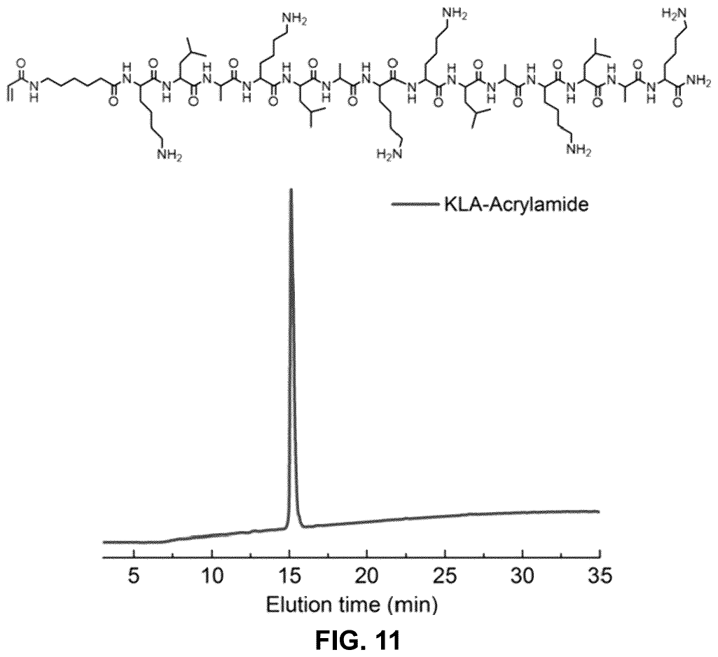

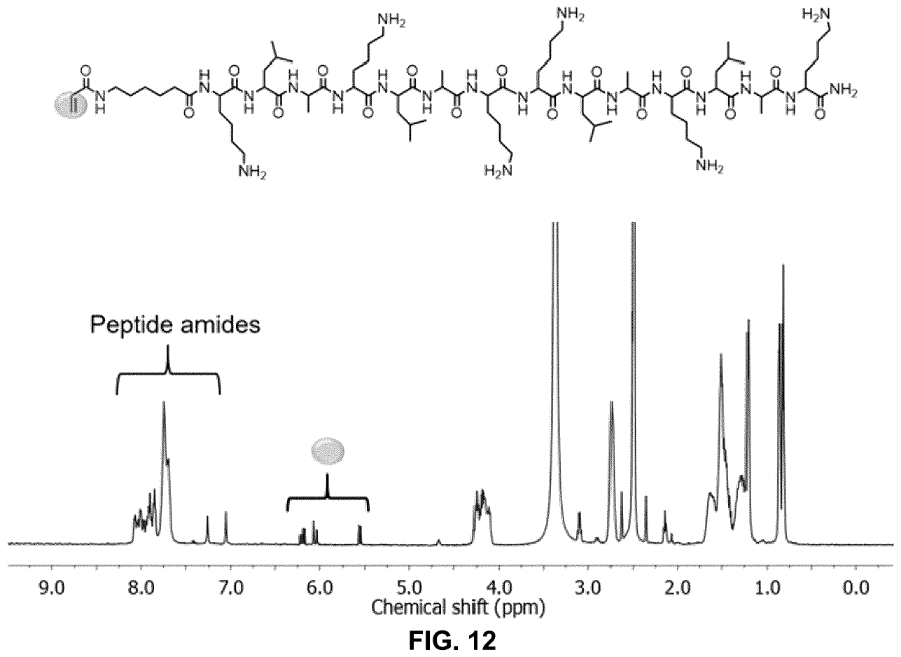

Two amino acid sequences were chosen to prepare two proof-of-concept systems. The first sequence GPLGLAGG (SEQ ID NO:5), is a known substrate for various proteolytic enzymes including thermolysin.^{[19]} The other is a sequence KLAKLAKKLAKLAK (SEQ ID NO:3), which, when internalized, triggers apoptosis of cells by mitochondrial membrane disruption. The chemical structure and purity of the peptide monomers (PepAm and KLAAm in FIG. 1) were verified by high performance liquid chromatography (HPLC), ¹H NMR spectroscopy, and electrospray ionization mass spectrometry (ESI-MS) (FIGS. 8-12).

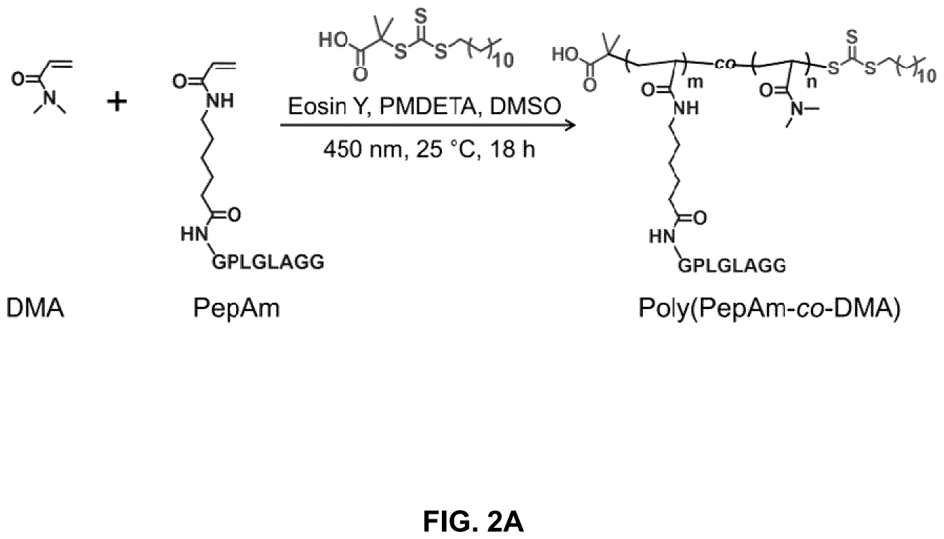

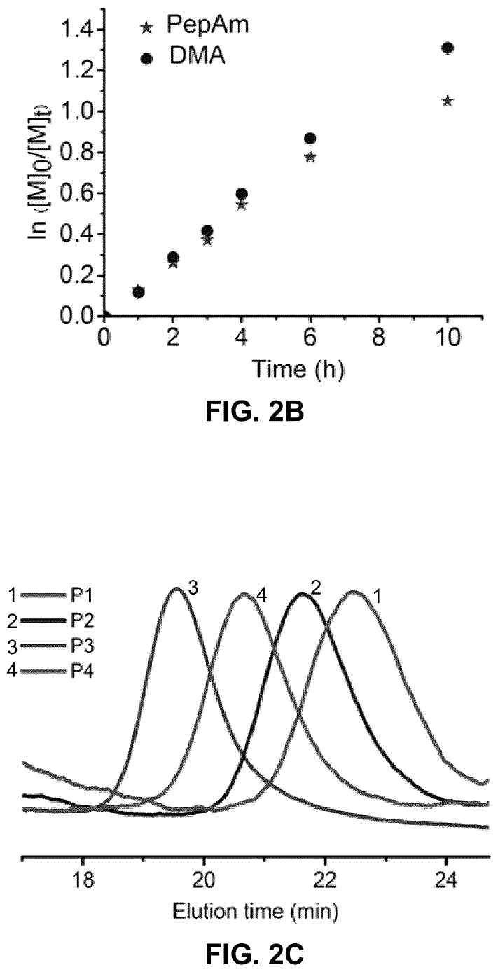

Due to the steric bulk of the peptide macromonomers, we reasoned that random copolymerization with a spacer monomer would enhance overall monomer conversions as well as improve control over the course of photo-RDRP. To examine this, the homopolymerization of PepAm in DMSO was first conducted (Table 1, Entry 1). According to the kinetics, no polymerization was observed after 18 hours, suggesting that steric hindrance stemming from the peptide side chains significantly hampered the photo-RDRP process. In view of this, a comonomer, dimethyl acrylamide was employed in the preparation of enzyme-responsive polypeptide brushes (Table 1, Entries 2-5). DMA was chosen due to its similar vinyl substructure (i.e., acrylamide) in comparison with the peptide monomer (FIGS. 2A-2C).

TABLE 1
Photo-RDRP of peptide-acrylamide (PepAm) and DMA

| | Equiv. to CTA | | | Conv. (%) | | M_{n, theo} | M_{n, GPC} | |
|---|---|---|---|---|---|---|---|---|
| Entry | PepAm | DMA | [M]₀ | PepAm | DMA | (g/mol) | (g/mol) | Ð |
| ^{a}P0 | 50 | 0 | 0.18M | 0% | N/A | N/A | N/A | N/A |
| ^{a}P1 | 50 | 50 | 0.36M | 12% | 15% | 5 600 | 4 900 | 1.08 |
| ^{a}P2 | 50 | 100 | 0.54M | 30% | 45% | 16 500 | 10 800 | 1.15 |
| ^{a}P3 | 50 | 150 | 0.72M | 68% | 78% | 39 900 | 46 000 | 1.01 |
| ^{b}P4 | 50 | 150 | 0.72M | 42% | 47% | 24 200 | 25 000 | 1.02 |

Note:
In each polymerization, 200 μL of DMSO was used.
[M]/[CTA]/[EY]/[PMDETA] = X/1/0.05/1.
^{a}The polymerizations were triggered by blue LED (450 nm).
^{b}The polymerization was promoted under sunlight in Northwestern University.

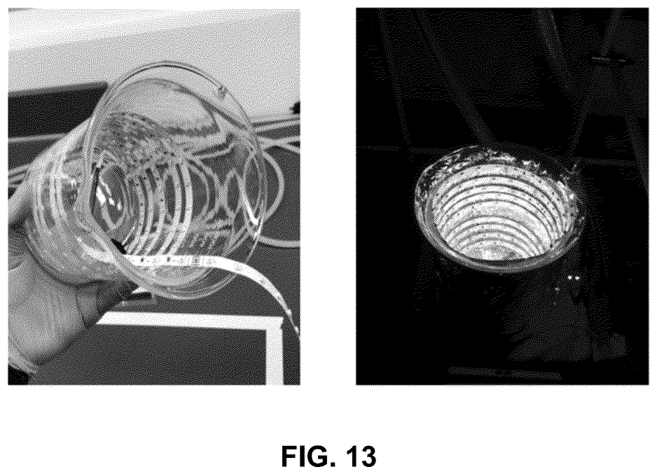

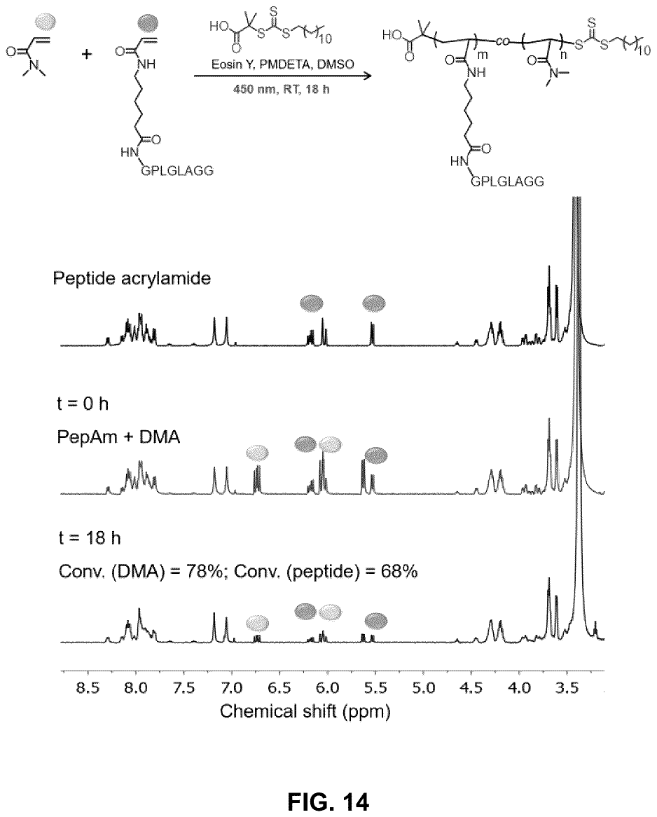

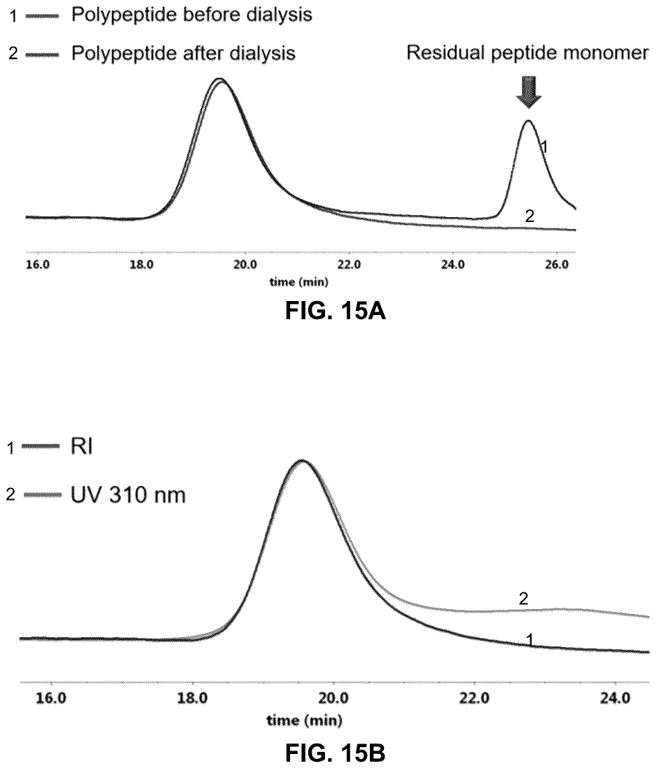

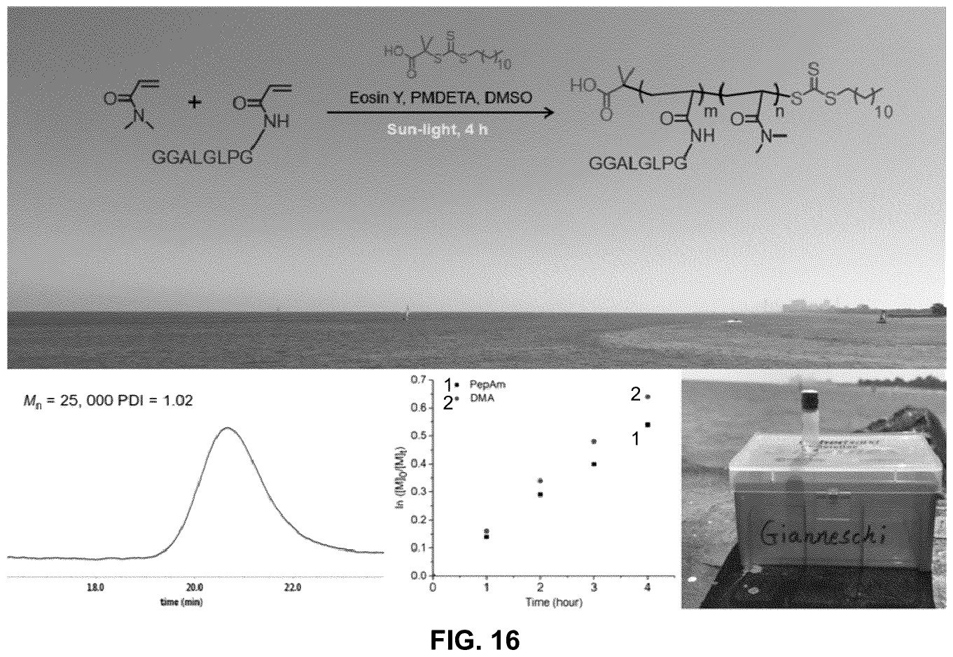

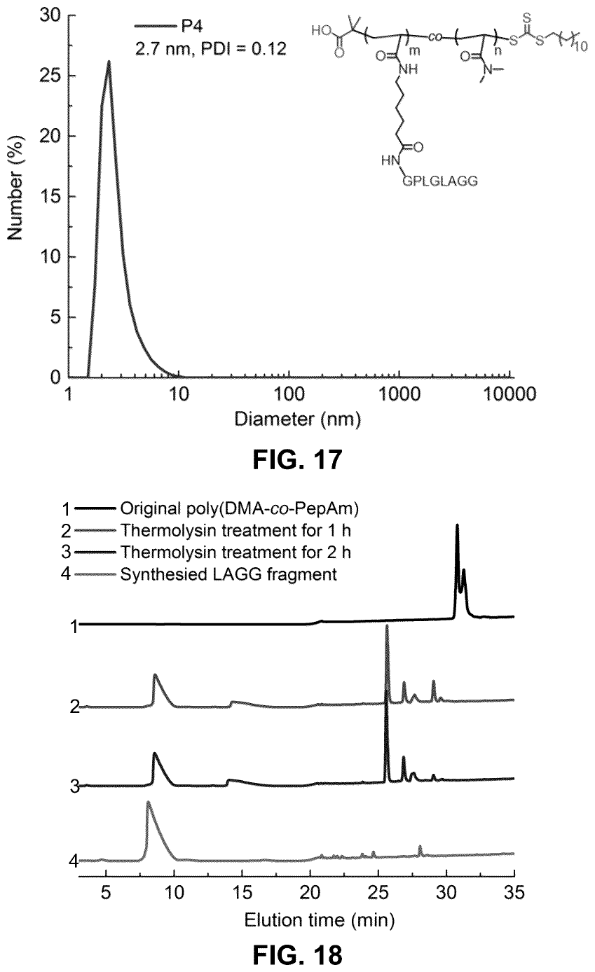

To understand the composition and distribution of PepAm and DMA in the polymers, ¹H NMR spectroscopy was used to study the rate of polymerization and conversion of the two monomers in photo-RDRP under blue LED (Table 1, entry 4, FIG. 13). According to ¹H NMR analysis (FIG. 2B and FIG. 14), both the peptide monomer and DMA comonomer had similar propagation rates, indicative of a statistical distribution of peptide monomers along the polymer backbone. Gel permeation chromatography (GPC) analysis showed a narrow molecular weight distribution for all polypeptide brushes with different grafting densities (FIG. 2C, Table 1). Moreover, theoretical molecular weights of all polymers are on par with those from GPC results, suggesting good control over the photo polymerizations. While full monomer conversions were not achieved, residual PepAm and DMA monomers were effectively removed by dialysis of the crude polymer mixtures in water, as confirmed by its disappearance in the GPC trace (FIGS. 15A-15B). We note, natural sun-light was also effective in triggering the photo-polymerization of PepAm, leading to well-defined polypeptide brushes (FIGS. 16-17).

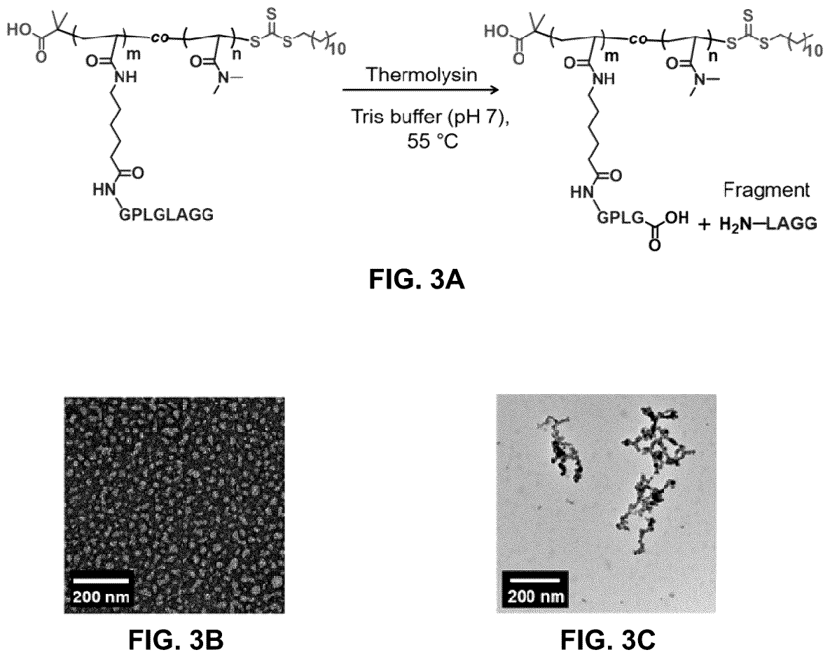

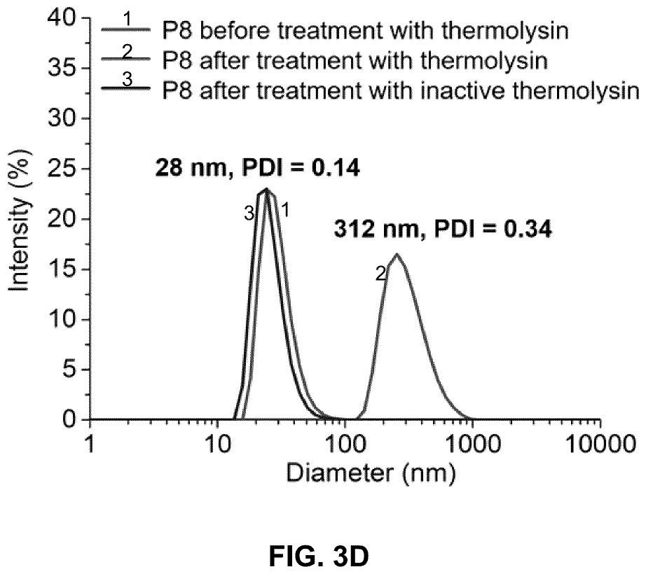

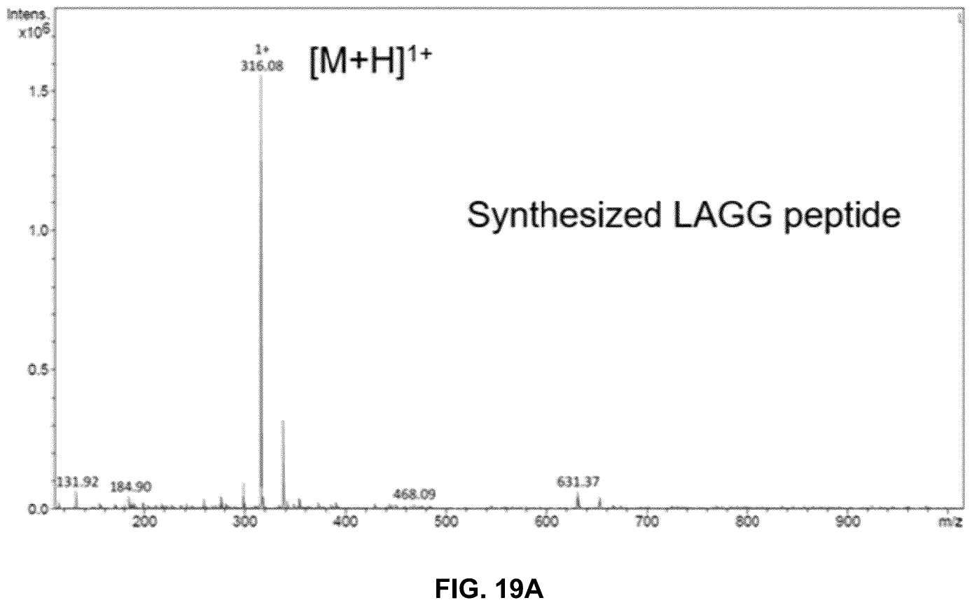

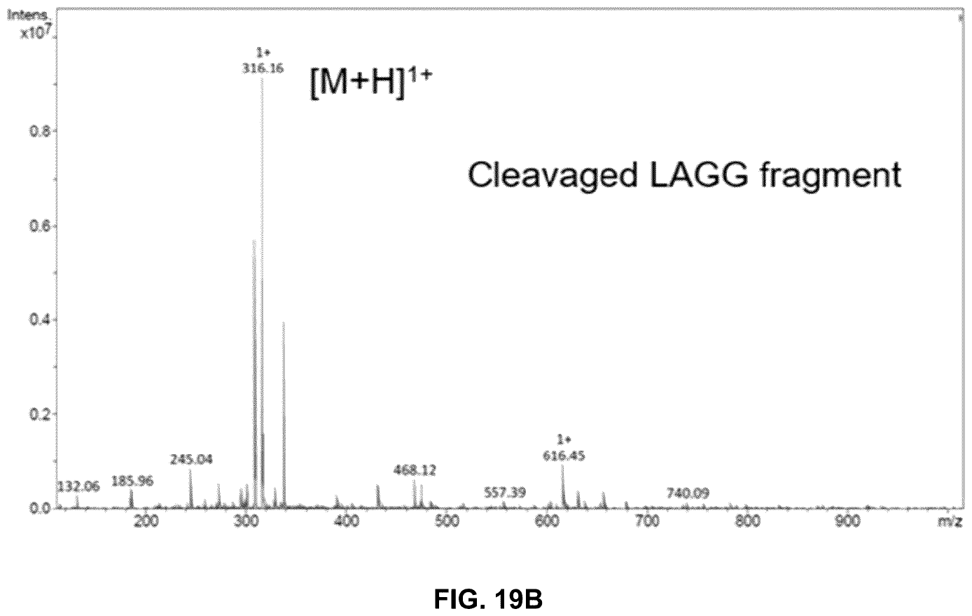

To examine the bio-activity of polypeptide brushes, polymer poly(PepAm₂₁-CO-DMA₇₁) (P4) was treated with thermolysin, an enzyme that can selectively cleave the amide bond between glycine (G) and leucine (L) (FIGS. 3A-3D). HPLC was employed to monitor the cleavage reaction, showing that the polypeptide brushes were rapidly cleaved within 1 hour under the investigated conditions (FIG. 18). The cleaved peptide fragment was further analyzed by ESI-MS and shown to have an identical mass to that of the genuine synthetic cleavage fragment LAGG (FIGS. 19A-19B). These results clearly indicate that the side-chain peptides remain accessible and reactive towards enzyme cleavage following polymerization. This is counter to our previous observations of highly dense peptide brushes generated using ring-opening metathesis polymerization, where peptides can be made entirely resistant to aggressive proteolytic treatments.^{[15]} The different activity of peptide brush polymers to enzyme digestion likely stems from the structural variation in polymer backbones prepared by photo-RDRP and ROMP. In comparison with rigid polynorbornene backbone containing sp² hybridized carbon-carbon double bonds, vinyl polymer backbone is more flexible and hence increases the accessibility of side chain peptides to surrounding enzymes. Moreover, the radical approach (i.e., photo-RDRP) to peptide brush polymers was achieved via copolymerization of peptide vinyl monomers and spacer monomers, resulting in random copolymers with a lower grafting density than that of polynorbornene-type polymers, which were synthesized by homopolymerization of norbornene modified peptide monomers.

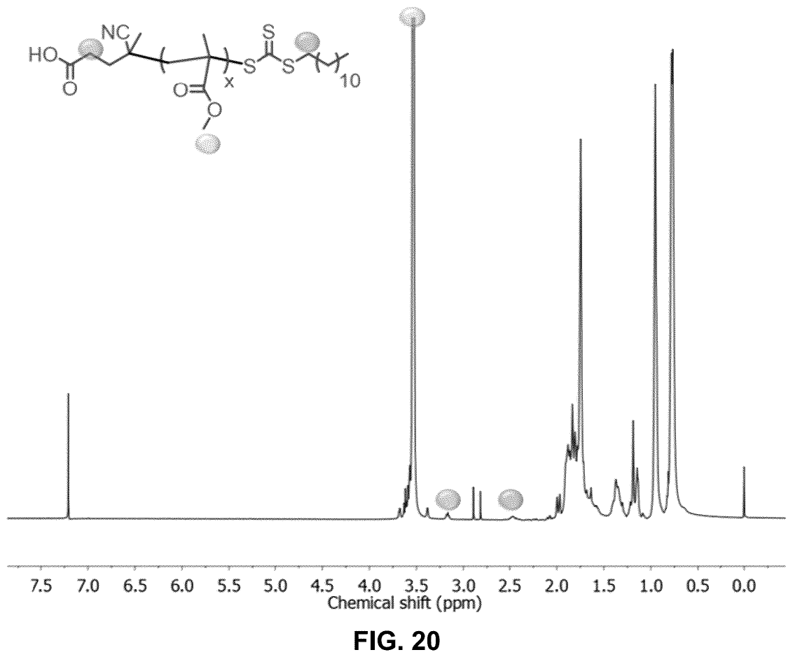

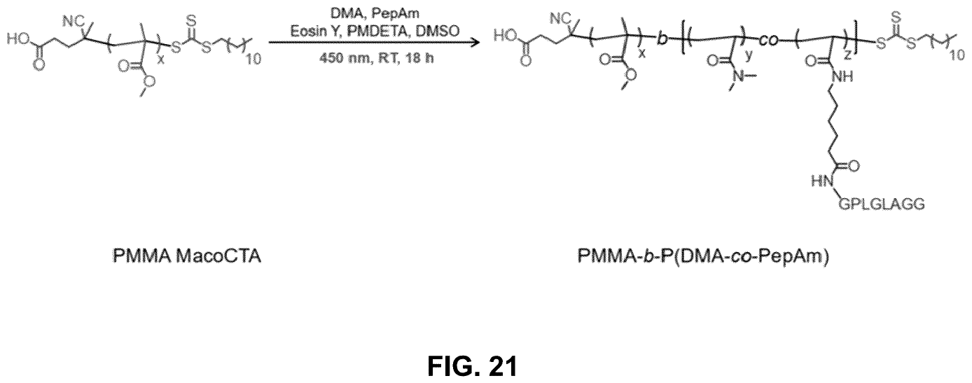

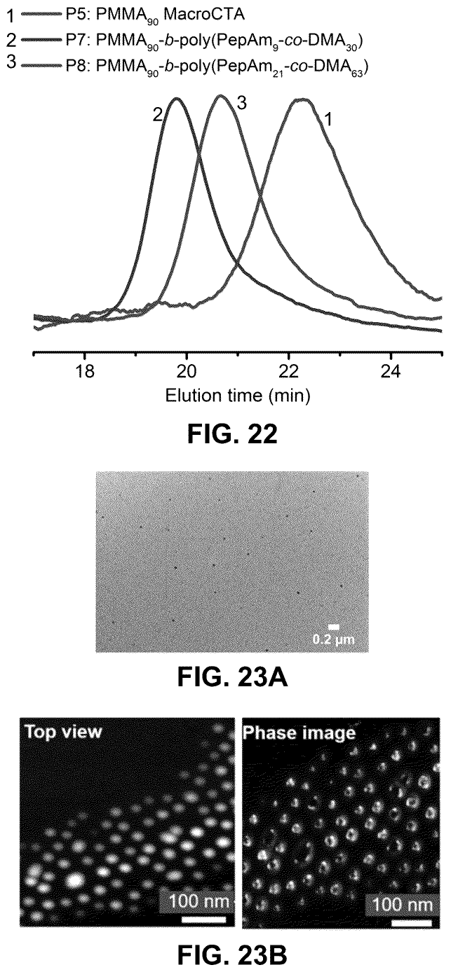

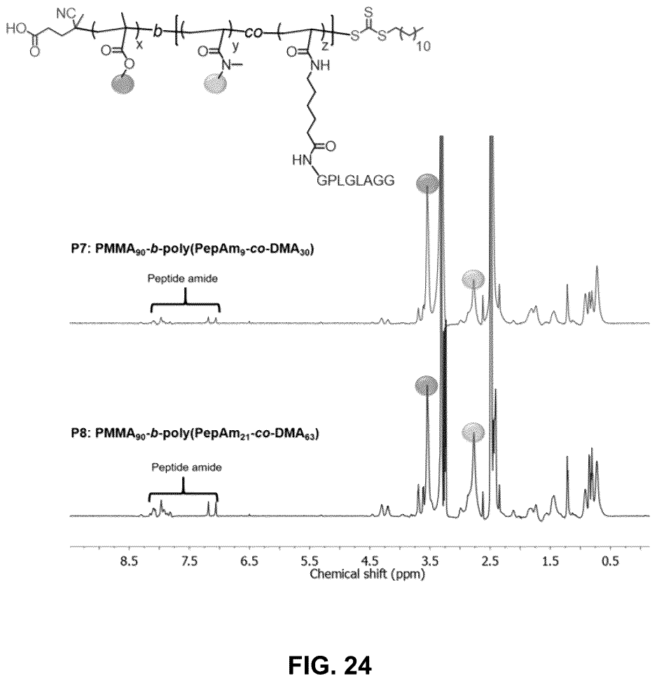

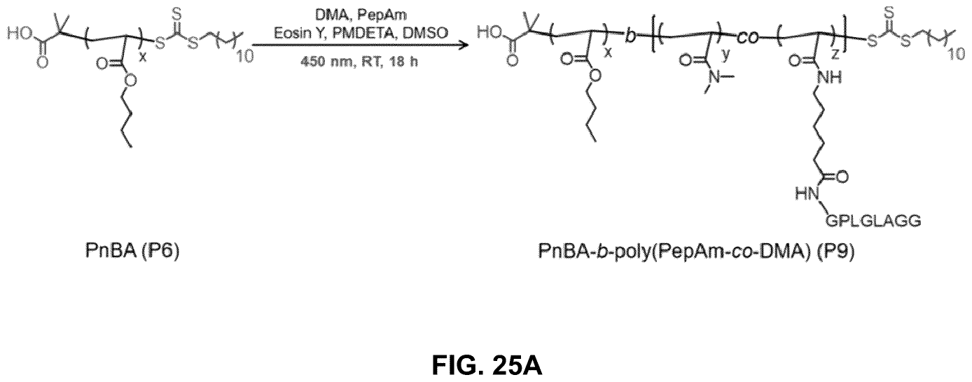

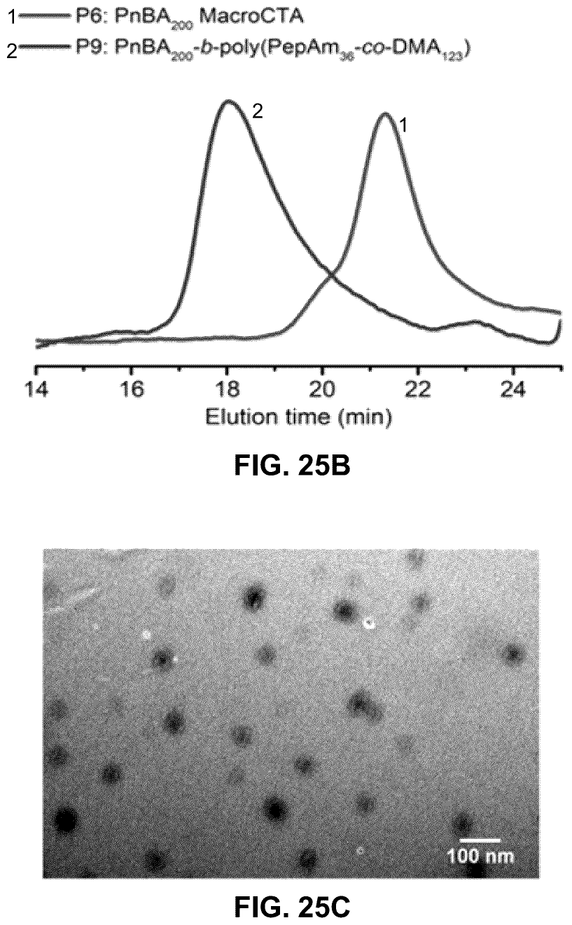

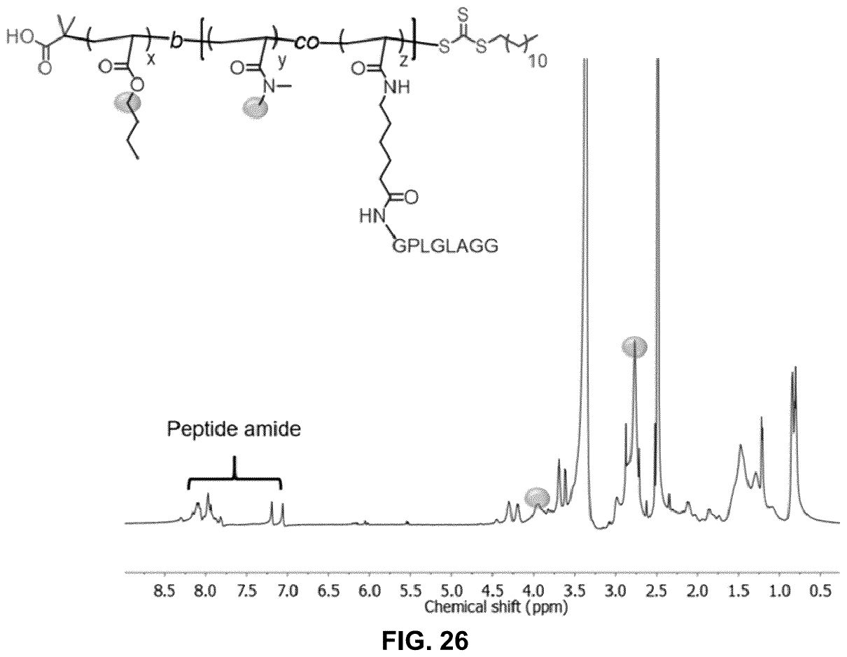

To capitalize on this accessibility to substrate, amphiphilic block copolymers were prepared (P7-P9, Table 2) by chain extension of poly(methyl methacrylate) or poly(n-butyl acrylate) based macro chain transfer agents with PepAm and DMA (FIGS. 20-26). The resulting amphiphilic diblock copolymers assembled into micelles in water. For example, PMMA₉₀-b-poly(PepAm₂₁-Co-DMA₆₃) (P8, Table 2) were spherical micelles, 24 nm in diameter as characterized by transmission electron microscope (TEM), in good agreement with the hydrodynamic diameter (28 nm) determined by dynamic light scattering (DLS) (FIGS. 3B and 3D).

TABLE 2
Preparation of enzyme-responsive diblock copolymer

| | Equiv. to macroCTA | | | Conv. (%) | | M_{n, theo} | M_{n, GPC} | |
|---|---|---|---|---|---|---|---|---|
| Entry | PepAm | DMA | [M]₀ | PepAm | DMA | (g/mol) | (g/mol) | Ð |
| ^{a}P7 | 13 | 37 | 0.72M | 72% | 79% | 19 900 | 16 200 | 1.04 |
| ^{a}P8 | 25 | 75 | 0.72M | 82% | 84% | 31 800 | 27 000 | 1.06 |
| ^{b}P9 | 50 | 150 | 0.72M | 71% | 82% | 68 000 | 66 400 | 1.17 |

Note:
In each polymerization, 200 μL of DMSO was used.
[M]/[CTA]/[EY]/[PMDETA] = X/1/0.05/1, RXN time = 18 h.
^{a}The chain extensions were performed using PMMA macroCTA (P5).
^{b}The chain extension was performed using PnBA macroCTA (P6).

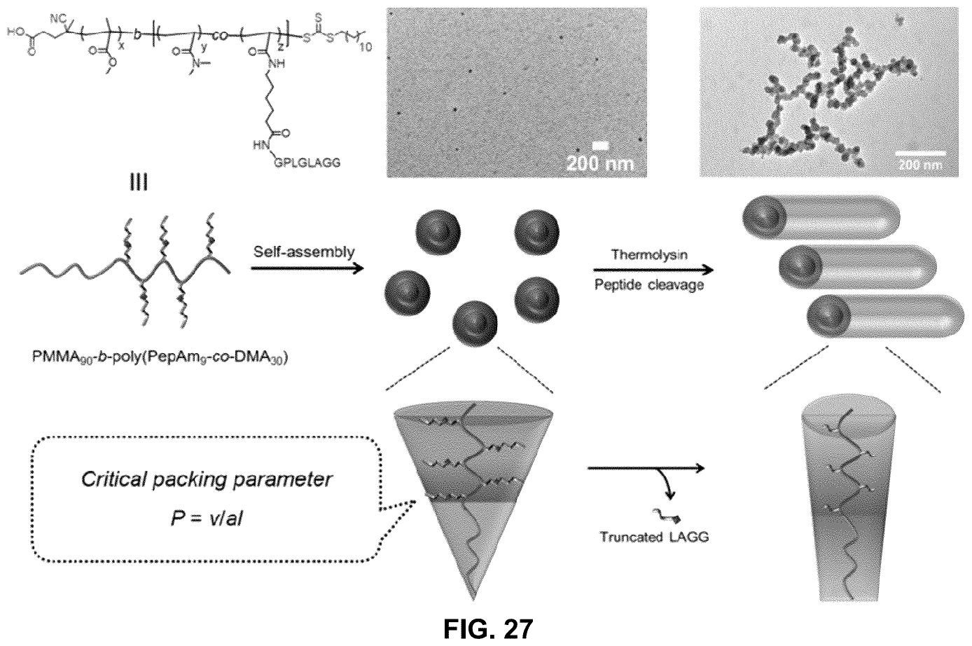

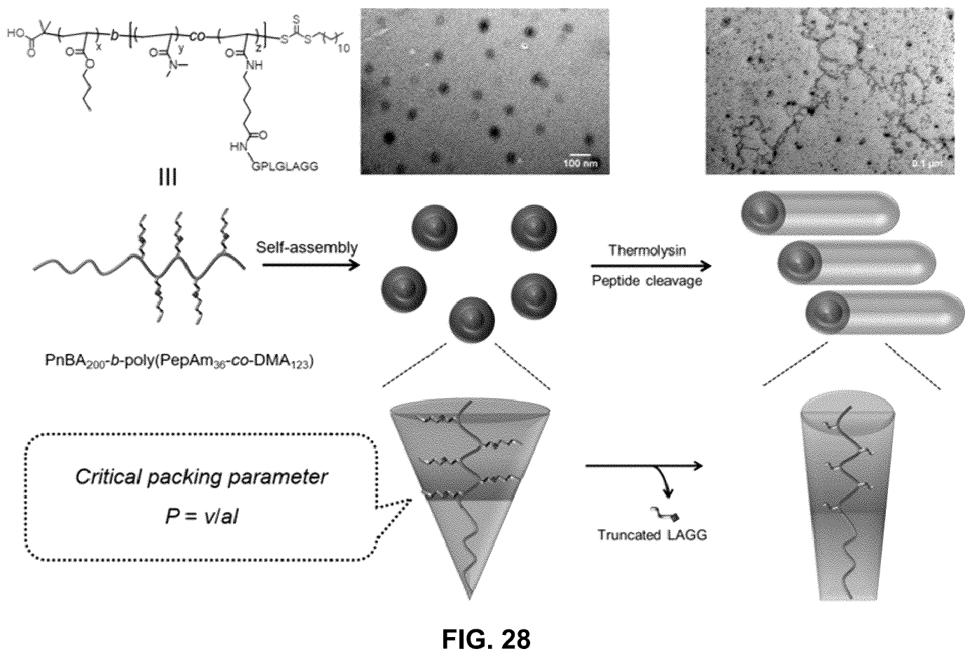

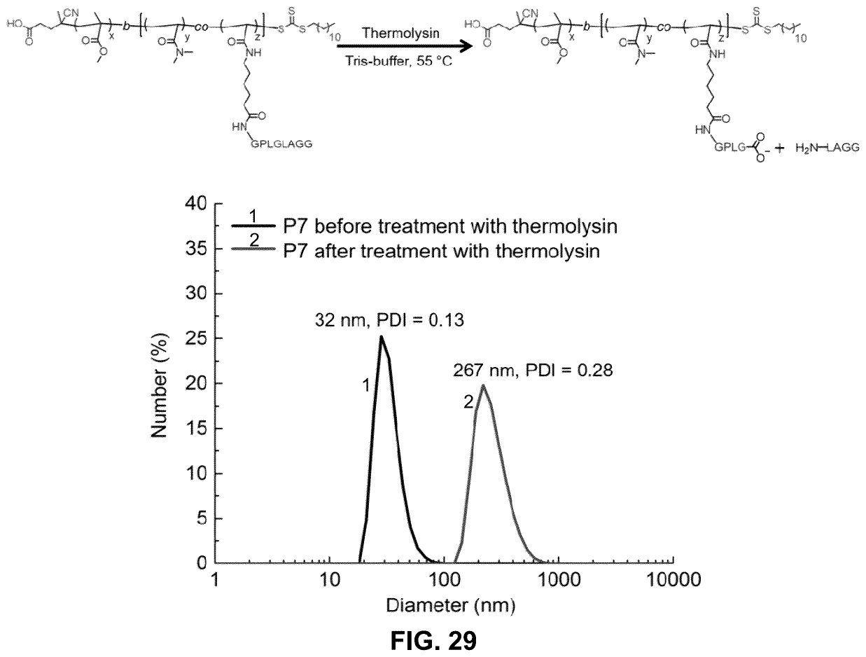

The critical packing parameter (CPP) dictates the thermodynamic morphology of amphiphilic block copolymers. In principle, a higher CPP (>1/3) can lead to the formation of higher order morphologies such as cylinders and bilayer vesicles.^{[23]} Since polypeptide brush polymer P4 showed rapid cleavage in the presence of thermolysin (vide supra), we expected that the micelles formed from polypeptide containing diblock polymers could respond to thermolysin, resulting in truncation of the hydrophilic polypeptide corona and thus a reduction in interfacial curvature (i.e., increment in CPP), leading to a change in morphology. Indeed, TEM and DLS showed that the spherical micellar structure of P8 underwent a phase transition into a worm-like phase upon treatment with thermolysin (FIGS. 3C and 3D). By contrast, no change in diameter was observed for particles treated under the same cleavage conditions using deactivated thermolysin which had been pretreated with ethylenediaminetetraacetic acid (FIG. 3D). In addition, similar morphological transformations were observed in other block copolymer micelles including PMMA₉₀-b-poly(PepAm₉-co-DMA₃₀) (P7) and PnBA₂₀₀-b-poly(PepAm₃₆-Co-DMA₁₂₃) (P9), demonstrating the versatility of this approach to enzyme-responsive shape-shifting nanoparticles (FIGS. 27-29).

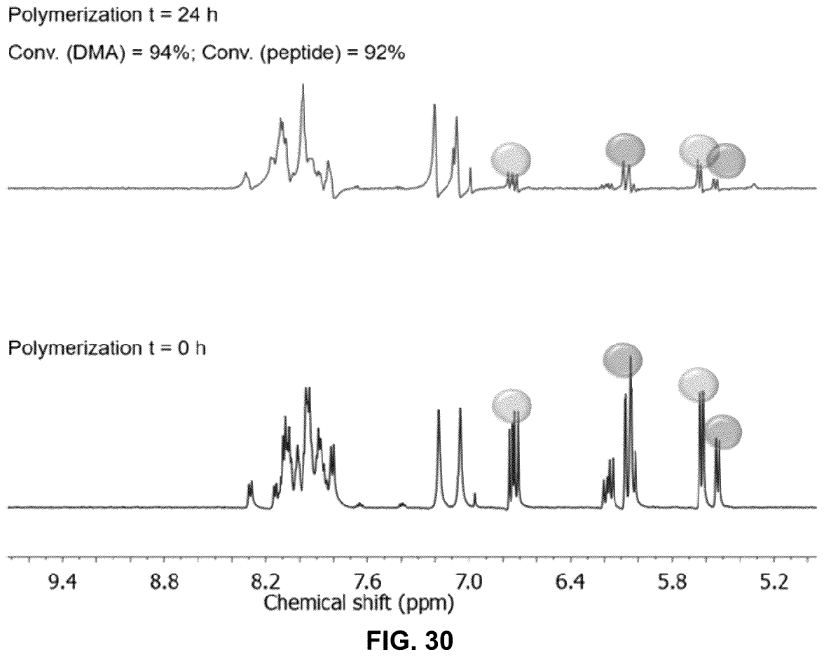

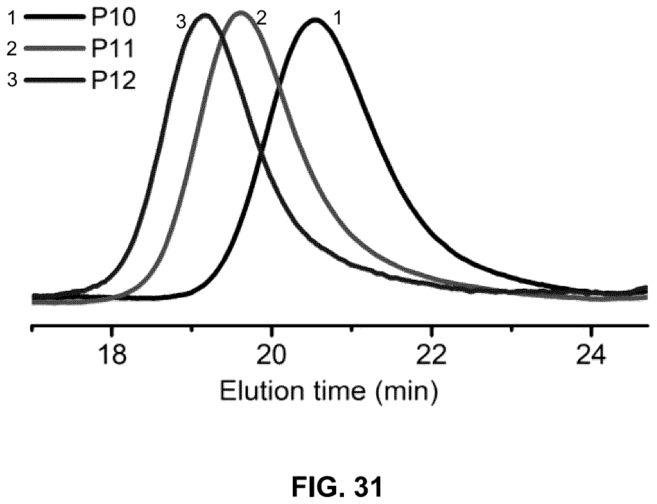

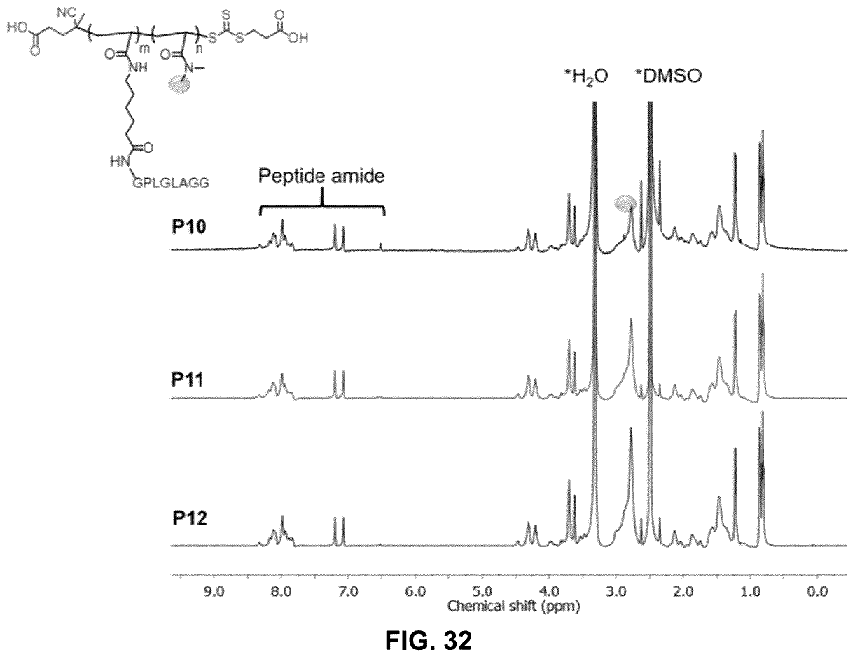

The ability to conduct polymerizations directly in water is of significant interest to the field of biomedical polymer materials, as it not only avoids the use of toxic organic solvents but also eliminates the time-consuming step of transferring the polymeric materials from the organic to aqueous phase. To explore the feasibility of aqueous photo-RDRP of peptide monomers, we examined the photo-RDRP of both enzyme-responsive peptide acrylamide (PepAm) and pro-apoptotic KLA peptide acrylamide (KLAAm) in water. Table 3 summarizes the polymerization results for PepAm and DMA, indicating dramatically higher monomer conversions in water compared to those obtained by photo-polymerizations in DMSO (Table 1). We hypothesize that this is due to the hydrogen bonding between the amide carbonyl groups with water molecules, leading to enhanced solubility of PepAm in aqueous solution.^{[24]} Polymers (P10-P12) were analyzed by GPC and NMR (FIGS. 30-32) and showed molecular weights that were in good agreement with theoretical values, confirming that photo-RDRP of PepAm was unaffected under aqueous conditions.

TABLE 3
Aqueous photo-RDRP of PepAm and DMA

| | Equiv. to CTA | | | Conv. (%) | | M_{n, theo} | M_{n, GPC} | |
|---|---|---|---|---|---|---|---|---|
| Entry | PepAm | DMA | [M]₀ | PepAm | DMA | (g/mol) | (g/mol) | Ð |
| ^{a}P10 | 50 | 50 | 0.36M | 60% | 64% | 27 685 | 26 100 | 1.06 |
| ^{a}P11 | 50 | 100 | 0.54M | 87% | 85% | 44 230 | 48 700 | 1.01 |
| ^{a}P12 | 50 | 150 | 0.72M | 92% | 94% | 51 388 | 55 400 | 1.04 |

Note:
In each polymerization, 200 μL of DMSO was used.
[M]/[CTA]/[EY]/[PMDETA] = X/1/0.05/1.
^{a}The polymerizations were triggered by blue LED.

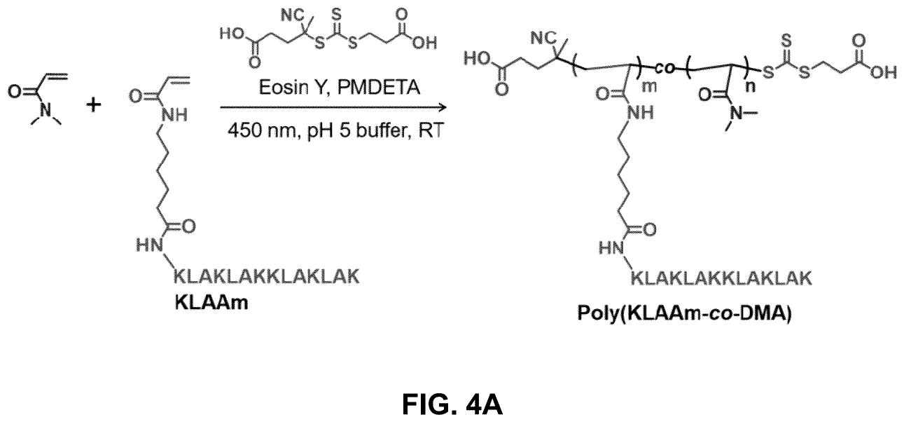

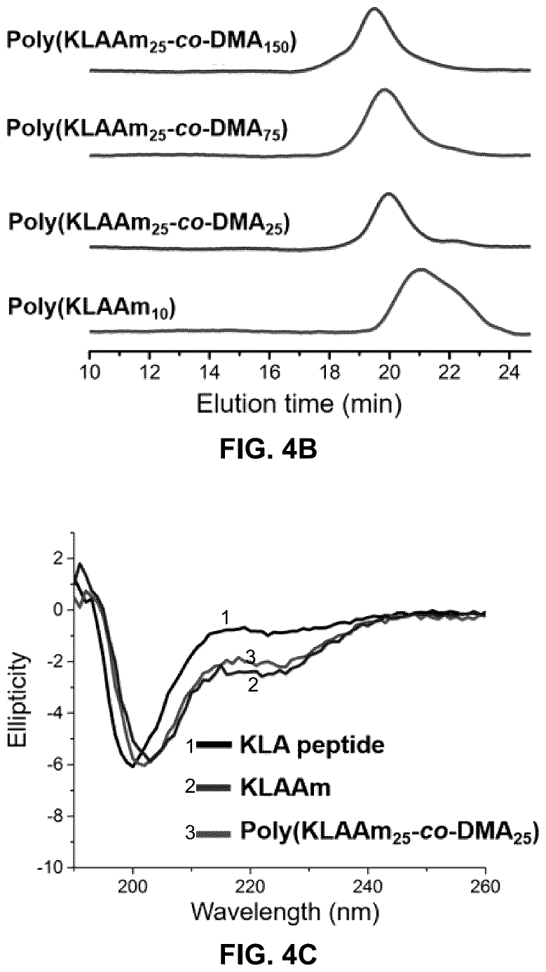

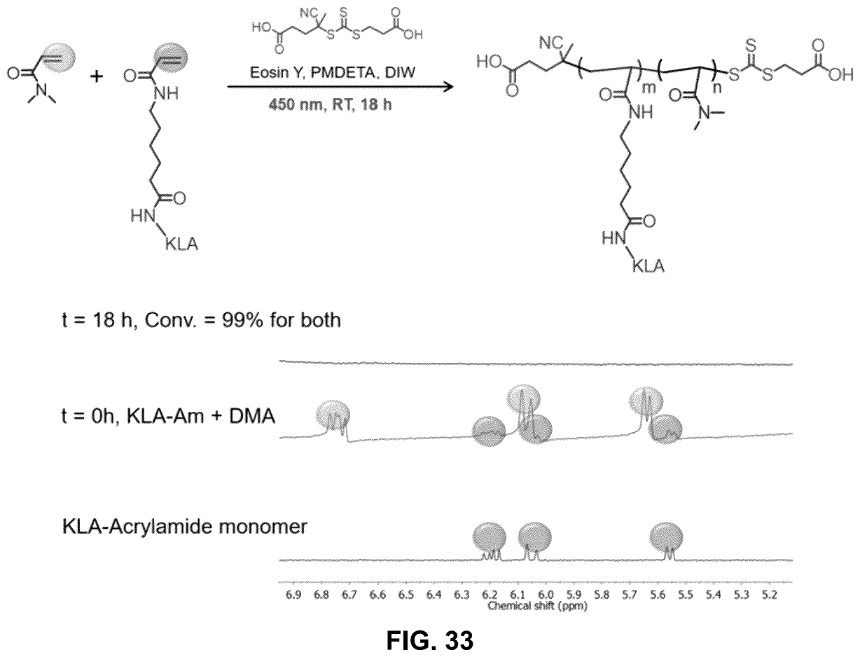

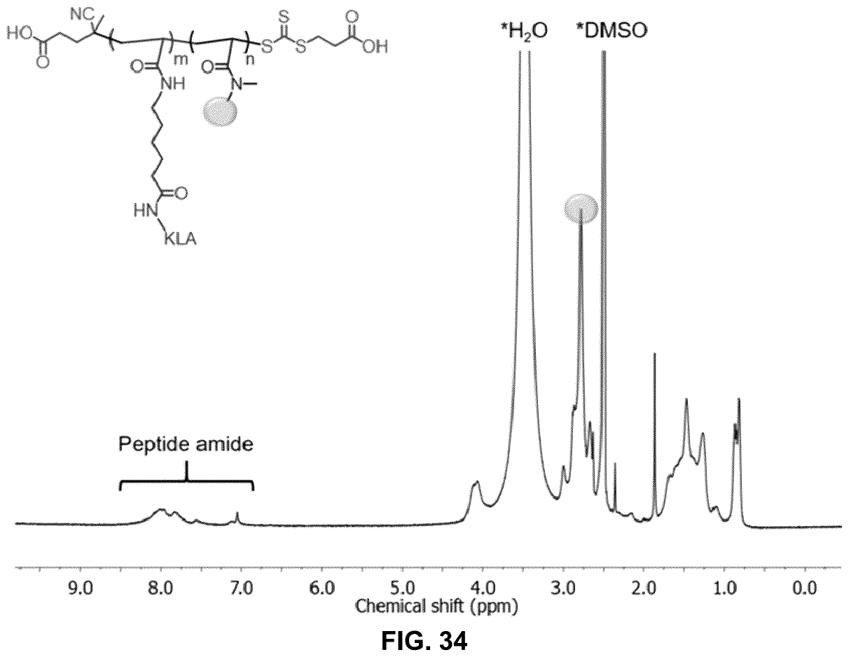

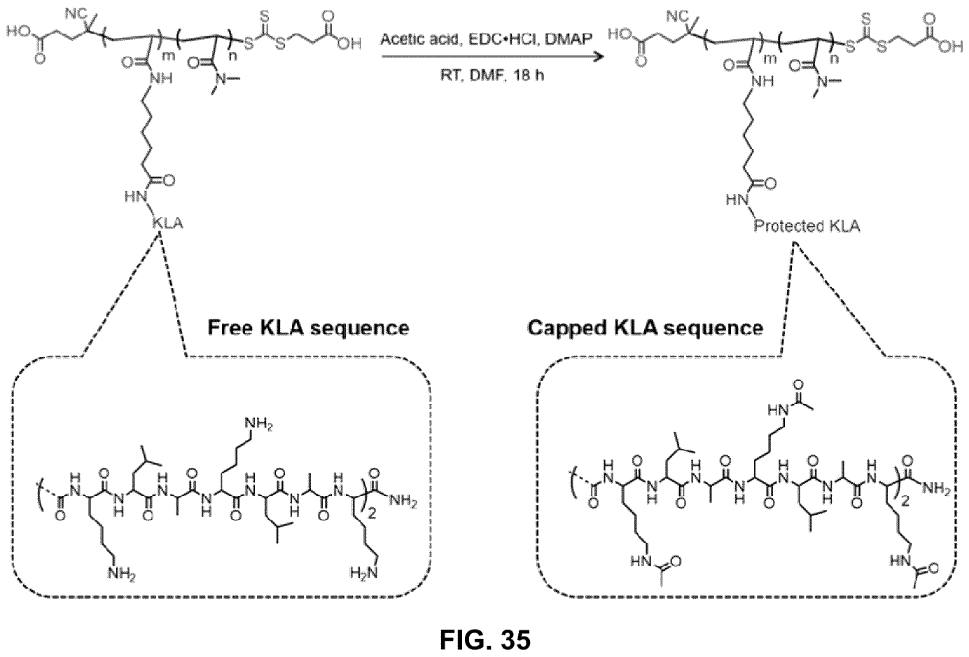

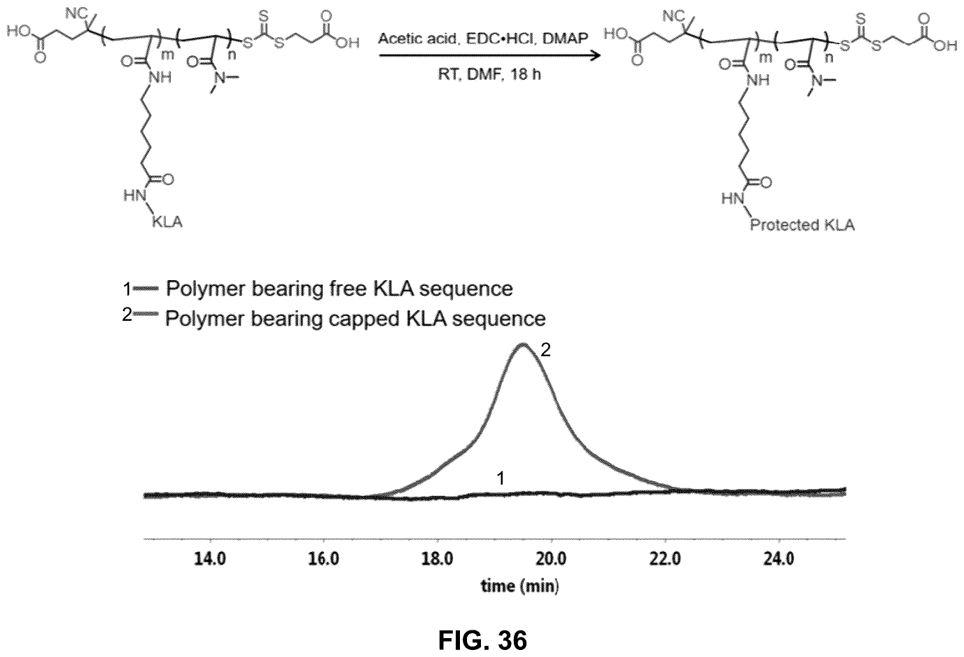

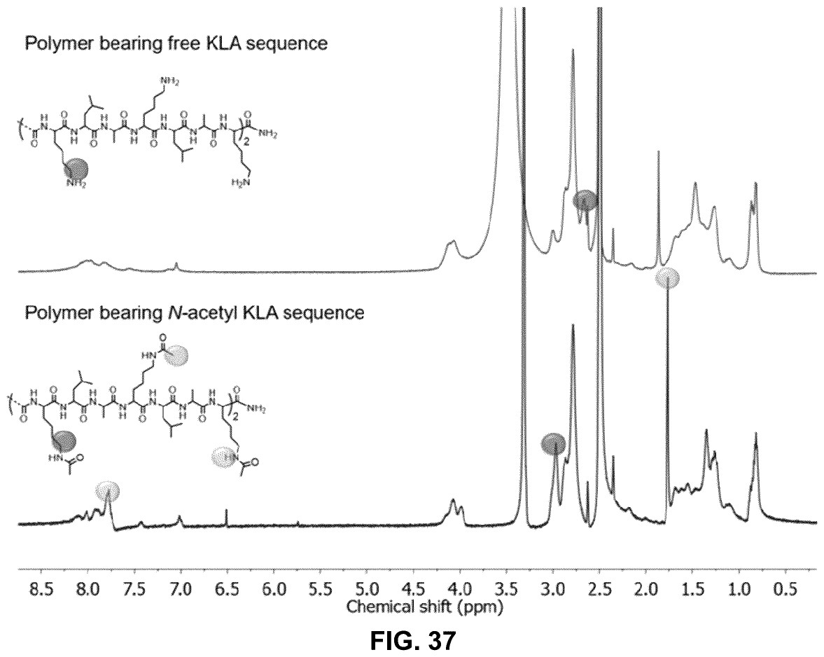

For the KLAAm monomer, which contains abundant amine groups, acetate buffer (pH 5) was utilized to fully protonate the amine groups, reducing their nucleophilicity (pKa=9) and thus precluding undesired aminolysis of the chain transfer agents (FIGS. 4A-4C). By adjusting the feed ratio of DMA to KLAAm, polypeptide brushes with various grafting densities of KLA side chains were prepared (Table 4, P13-P16). Based on NMR analysis, monomer conversions were quantitative for all random copolymerizations of DMA and KLAAm (FIGS. 33-34). However, the homopolymerization of KLAAm led to a modest monomer conversion (40%) possibly due to the steric bulk of the KLA peptide macromonomer. The narrow and symmetric GPC traces of KLA based polypeptide brushes indicates good control over the aqueous photo-RDRP of KLAAm (FIG. 4B, FIGS. 36-37). Furthermore, the secondary structure of KLA peptides and brush polymers were assessed by circular dichroism (CD) spectroscopy, which showed a mixture of α-helix and random coil. The CD spectra of the KLA monomer and resulting polymers are identical, suggesting the polymerization process does not alter the secondary structure of the peptide (FIG. 4C).

TABLE 4
Aqueous photo-RDRP of KLA-acrylamide (KLAAm) and DMA

| | Equiv. to CTA | | | Conv. (%) | | M_{n, theo} | M_{n, GPC} | |
|---|---|---|---|---|---|---|---|---|
| Entry | KLAAm | DMA | [M]₀ | KLAAm | DMA | (g/mol) | (g/mol) | Ð |
| ^{a}P13 | 25 | 0 | 0.09M | 40% | N/A | 17 207 | 24 100 | 1.09 |
| ^{a}P14 | 25 | 25 | 0.18M | 98% | 99% | 45 032 | 48 200 | 1.03 |
| ^{a}P15 | 25 | 75 | 0.36M | 99% | 99% | 49 982 | 53 600 | 1.08 |
| ^{a}P16 | 25 | 150 | 0.72M | 99% | 99% | 57 407 | 62 400 | 1.16 |

Note:
In each polymerization, 200 μL of acetate buffer (0.1M, pH 5) was used.
[M]/[CTA]/[EY]/[PMDETA] = X/1/0.05/1.
^{a}The polymerizations were triggered by blue LED.

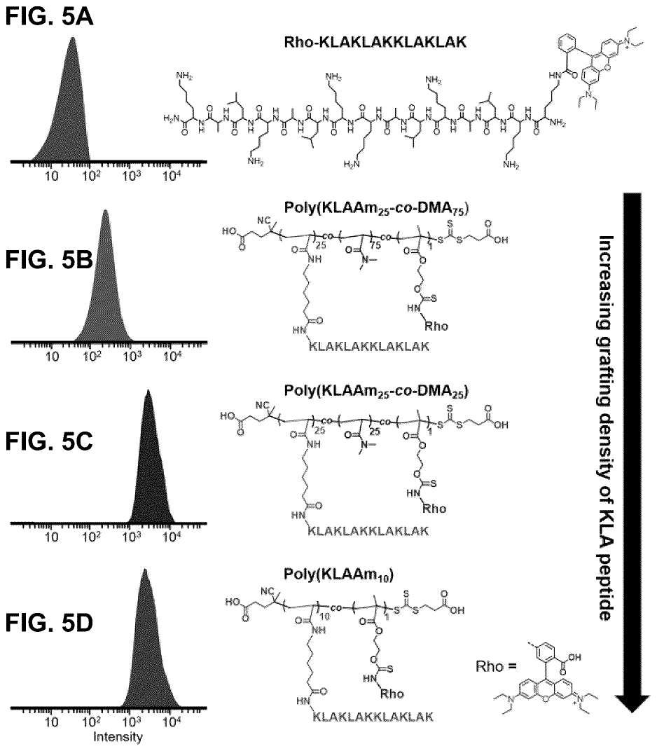

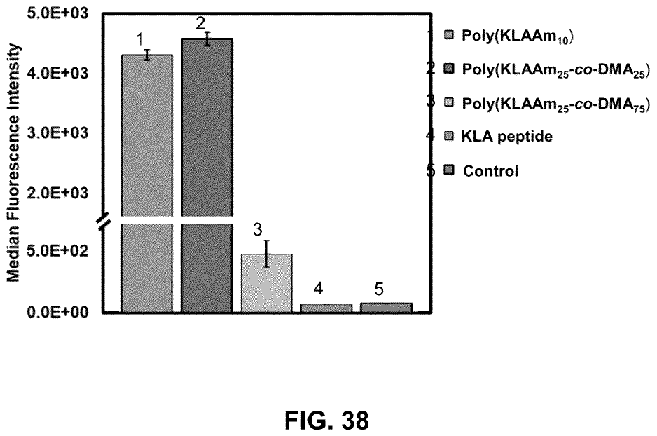

The KLA peptide sequence used in these studies is a known pro-apoptotic peptide which is capable of inducing cell apoptosis via disruption of mitochondrial membranes.^{[25]} It is typically fused with a cell-penetrating peptide because of its otherwise poor cellular uptake.^{[26]} The KLA based brush polymers, while lacking a cell-penetrating peptide moiety, collectively possess a number of cationic charges when polymerized, which could enhance the affinity to the negatively charged cell membrane and consequently promote delivery of the KLA based polymer brushes into cells.^{[16]} To elucidate the role of KLA grafting densities on the cellular uptake and cytotoxicity of KLA brush polymers, we conducted in vitro cell studies of different brush polymers (P13-P16, Table 4) in Hela cells (FIGS. 5-7). Flow cytometry was used to quantify the cell uptake efficiency of brush polymers. All KLA peptide brush polymers show significantly more cellular uptake compared to the free peptide (FIGS. 5A-5D). This observation is consistent with the multivalency of KLA peptides organized as polypeptide brushes, which enhanced the affinity and cell uptake of the KLA containing materials. In addition, cell internalization of densely grafted polypeptides including poly(KLAAm₂₅-CO-DMA₂₅) and poly(KLAAm₁₀) clearly outperformed more sparsely grafted polypeptide brushes such as poly(KLAAm₂₅-CO-DMA₇₅) (FIGS. 5A-5D and 38).

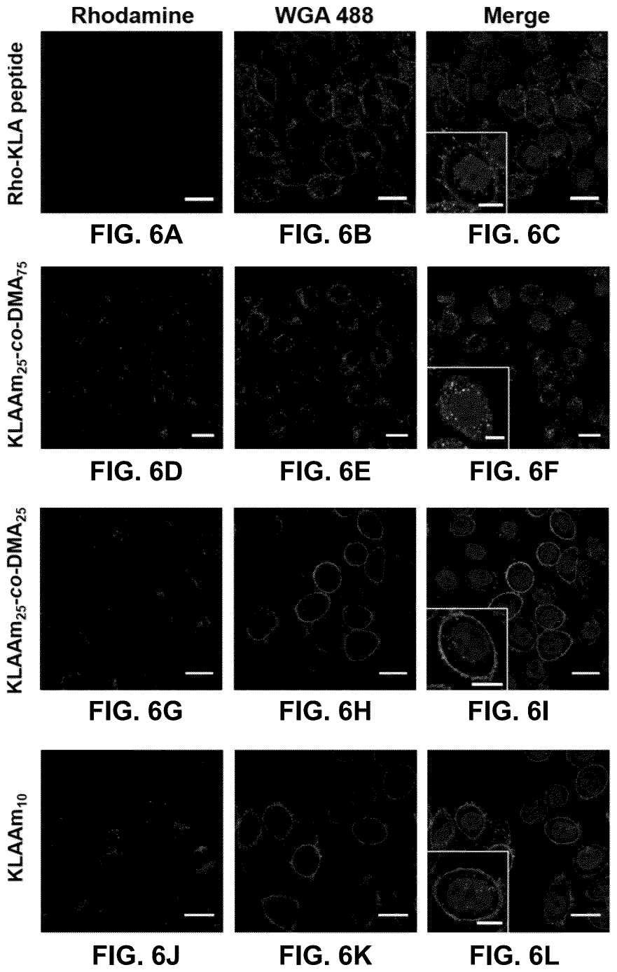

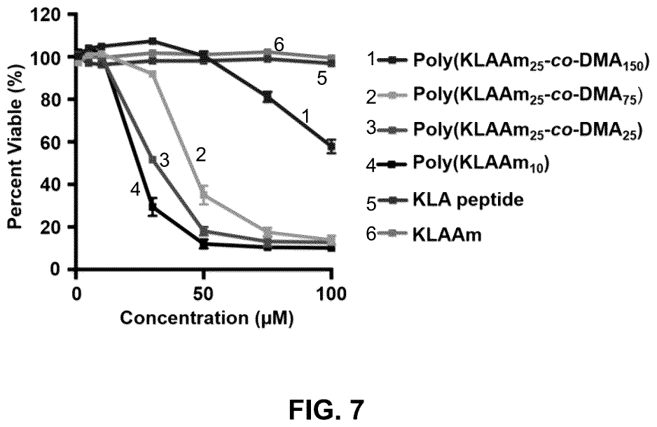

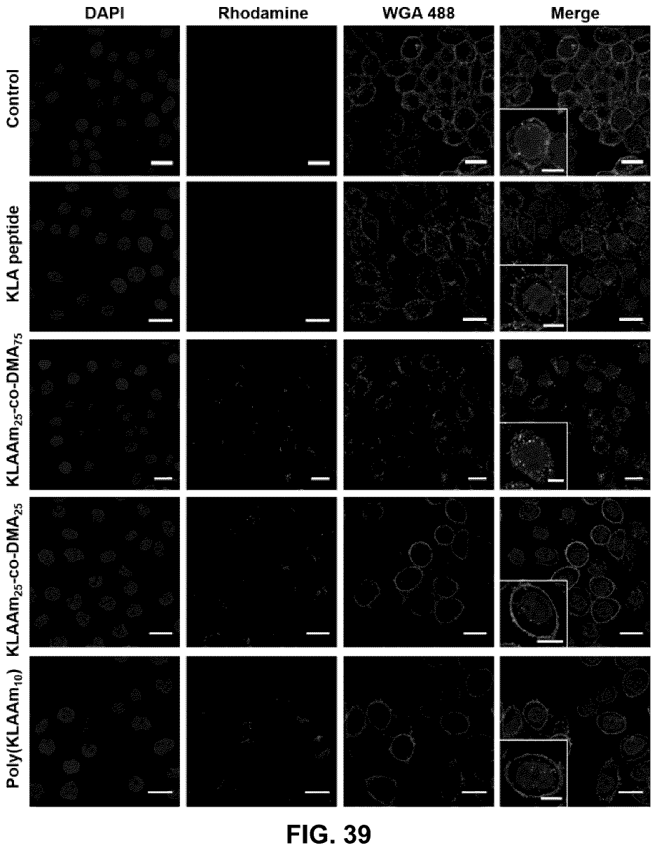

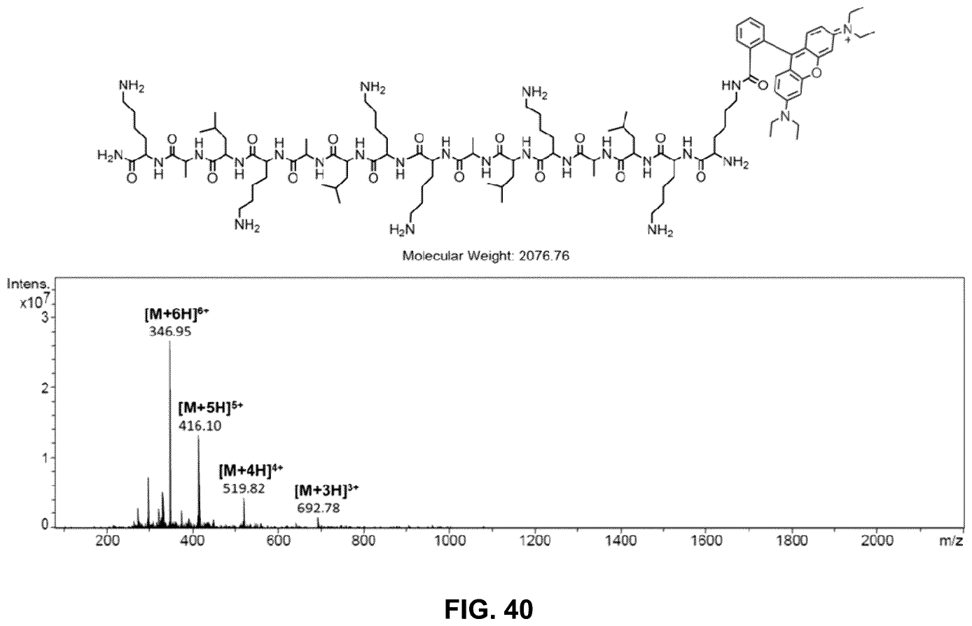

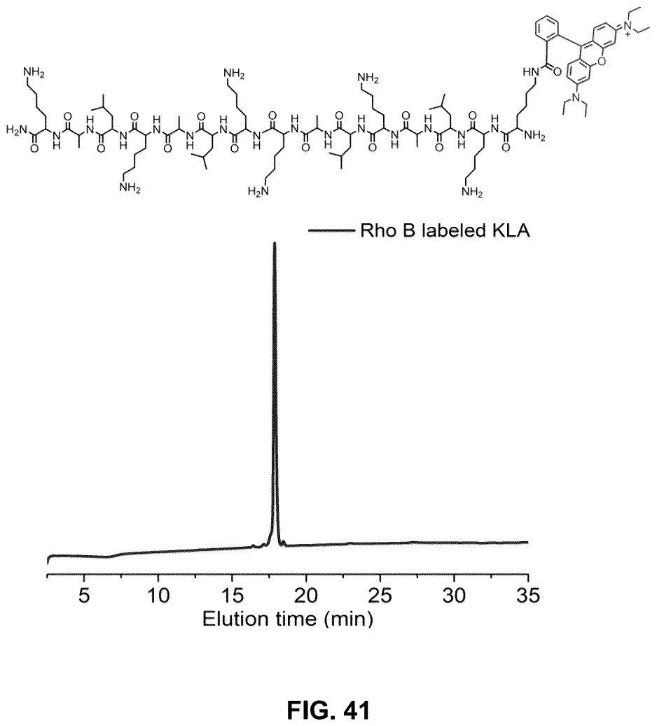

The cellular uptake behavior of KLA peptide and polymer brushes were further studied by confocal laser scanning microscopy (CLSM). Cells treated with rhodamine labeled KLA peptide showed no uptake even at a high concentration (50 μM with respect to peptide). On the other hand, the cellular uptake of all KLA peptide brush polymers was clearly visible at the same peptide concentration, as evidenced by the increase in rhodamine fluorescence in Hela cells (FIGS. 6A-6L and 39). Finally, cytotoxicity assays demonstrated that KLA based polymer brushes had significantly higher cytotoxicity than either the free KLA peptide or the KLAAm monomer (FIG. 7). Notably, the cytotoxicity of polypeptide brushes was dependent on the grafting density of KLA peptides. As the grafting density of KLA peptide increased and the polymer brushes became more compact, the half-maximum inhibitory concentration IC₅₀ values decreased. These cytotoxicity results are consistent with the observed cellular uptake behavior of KLA brush polymers, further demonstrating the role of grafting density on the material properties.

Conclusion: In summary, we present examples of photo-RDRP of peptide acrylamide monomers. This is a robust synthetic approach to prepare bioactive polypeptide brushes under mild conditions using visible light, in aqueous solution, and at room temperature. We envision that a wide variety of other functional peptide monomers such as therapeutic and cell-penetrating peptides will be compatible with this technique. Moreover, we demonstrated the important role that the architecture (i.e., grafting density) of peptide brush polymers has on function such as cell penetration and cytotoxicity towards cancer cells. Given the widespread interest in peptides as therapeutics and targeting moieties in biomedicine, we envision these mild synthetic procedures will open the door to entirely new peptide brush polymer biomaterials.

### EXPERIMENTAL EXAMPLES

Preparation of Peptide Vinyl Monomers via Solid-Phase Peptide Synthesis (SPPS): peptides were synthesized on Rink resins (0.67 mmol/g) using standard FMOC SPPS procedures on an AAPPTec Focus XC automated synthesizer. A typical SPPS procedure included deprotection of the N-terminal Fmoc group with 20% 4-methyl-piperidine in DMF (1×20 min, followed by 1×5 min), and 30 min amide couplings (twice) using 3.0 equiv. of the Fmoc-protected amino acid, 2.9 equiv. of HBTU and 6.0 equiv. of DIPEA. After that, peptide acrylamide monomers were prepared by amide coupling to Fmoc-6-aminohexanoic acid, followed by Fmoc deprotection and final amidation with acrylic acid (3 equiv.) in the presence of HBTU (2.9 equiv.), and DIPEA (6.0 equiv.). The crude peptide monomers were obtained by cleavage from the resins and further purified by preparative HPLC.

Aqueous photo-RDRP of peptide acrylamide monomers: In a typical aqueous photoinduced polymerization (P14), KLA peptide (KLAKLAKKLAKLAK) (SEQ ID NO: 3) acrylamide monomer (30 mg, 25 equiv.) and DMA (1.8 mg, 25 equiv.) were dissolved in 150 μL of acetate buffer (0.1 M, pH 5). Then 10 μL (1.0 equiv.) of water-soluble RAFT agent stock solution (2.2 mg in 100 μL of acetate buffer) was added into the reaction mixture. Following that, 10 μL (0.05 equiv.) of eosin Y disodium salt stock solution (2.5 mg in 1 mL of acetate buffer) and PMDETA (0.12 mg, 1.0 equiv.) were added. The solution was degassed by N₂ flow for 30 min and then placed into the photo-reactor (450 nm, 2.8 mW/cm²) for 24 h. Upon the polymerization, the polymer product was purified by dialysis into DIW, followed by lyophilization.

### References corresponding to Example 1A

[1] a) N. Corrigan, J. Yeow, P. Judzewitsch, J. T. Xu, C. Boyer, Angew. Chem. Int. Ed. 2019, 58, 5170-5189; Angew. Chem. 2019, 131, 5224-5243; b) J. T. Xu, K. Jung, A. Atme, S. Shanmugam, C. Boyer, J. Am. Chem. Soc. 2014, 136, 5508-5519; c) M. Chen, M. J. Zhong, J. A. Johnson, Chem. Rev. 2016, 116, 10167-10211; d) H. Sun, C. P. Kabb, M. B. Sims, B. S. Sumerlin, Prog. Polym. Sci. 2019, 89, 61-75; e) M. R. Molla, P. Rangadurai, L. Antony, S. Swaminathan, J. J. de Pablo, S. Thayumanavan, Nat. Chem. 2018, 10, 659-666; f) L. Yang, H. Sun, Y. Liu, W. J. Hou, Y. Yang, R. Cai, C. Cui, P. H. Zhang, X. S. Pan, X. W. Li, L. Li, B. S. Sumerlin, W. H. Tan, Angew. Chem. Int. Ed. 2018, 57, 17048-17052; Angew. Chem. 2018, 130, 17294-17298.
[2] J. Niu, D. J. Lunn, A. Pusuluri, J. I. Yoo, M. A. O'Malley, S. Mitragotri, H. T. Soh, C. J. Hawker, Nat. Chem. 2017, 9, 537-545.
[3] a) X. C. Pan, S. Lathwal, S. Mack, J. J. Yan, S. R. Das, K. Matyjaszewski, Angew. Chem. Int. Ed. 2017, 56, 2740-2743; Angew. Chem. 2017, 129, 2784-2787; b) X. C. Pan, C. Fang, M. Fantin, N. Malhotra, W. Y. So, L. A. Peteanu, A. A. Isse, A. Gennaro, P. Liu, K. Matyjaszewskit, J. Am. Chem. Soc. 2016, 138, 2411-2425.
[4] R. N. Carmean, T. E. Becker, M. B. Sims, B. S. Sumerlin, Chem 2017, 2, 93-101.
[5] J. T. Xu, S. Shanmugam, C. K. Fu, K. F. Aguey-Zinsou, C. Boyer, J. Am. Chem. Soc. 2016, 138, 3094-3106.
[6] J. B. Tan, H. Sun, M. G. Yu, B. S. Sumerlin, L. Zhang, ACS Macro Lett. 2015, 4, 1249-1253.
[7] K. A. Ogawa, A. E. Goetz, A. J. Boydston, J. Am. Chem. Soc. 2015, 137, 1400-1403.
[8] a) V. Kottisch, M. J. Supej, B. P. Fors, Angew. Chem. Int. Ed. 2018, 57, 8260-8264; Angew. Chem. 2018, 130, 8392-8396; b) Q. Michaudel, V. Kottisch, B. P. Fors, Angew. Chem. Int. Ed. 2017, 56, 9670-9679; Angew. Chem. 2017, 129, 9798-9808; c) V. Kottisch, Q. Michaudel, B. P. Fors, J. Am. Chem. Soc. 2016, 138, 15535-15538.
[9] a) F. Eisenreich, M. Kat han, A. Dallmann, S. P. Ihrig, T. Schwaar, B. M. Schmidt, S. Hecht, Nat. Catal. 2018, 1, 516-522; b) C. K. Fu, J. T. Xu, C. Boyer, Chem. Commun. 2016, 52, 7126-7129.
[10] G. Ng, J. Yeow, R. Chapman, N. Isahak, E. Wolyetang, J. J. Cooper-White, C. Boyer, Macromolecules 2018, 51, 7600-7607.
[11] Q. Z. Yang, J. Lalevee, J. Poly, Macromolecules 2016, 49, 7653-7666.
[12] S. Varlas, L. D. Blackman, H. E. Findlay, E. Reading, P. J. Booth, M. I. Gibson, R. K. O'Reilly, Macromolecules 2018, 51, 6190-6201.
[13] L. D. Blackman, S. Varlas, M. C. Arno, Z. H. Houston, N. L. Fletcher, K. J. Thurecht, M. Hasan, M. I. Gibson, R. K. O'Reilly, ACS Central Sci. 2018, 4, 718-723.
[14] M. R. Hill, R. N. Carmean, B. S. Sumerlin, Macromolecules 2015, 48, 5459-5469.
[15] A. P. Blum, J. K. Kammeyer, J. Yin, D. T. Crystal, A. M. Rush, M. K. Gilson, N. C. Gianneschi, J. Am. Chem. Soc. 2014, 136, 15422-15437.
[16] A. P. Blum, J. K. Kammeyer, N. C. Gianneschi, Chem. Sci. 2016, 7, 989-994.
[17] H. D. Maynard, S. Y. Okada, R. H. Grubbs, J. Am. Chem. Soc. 2001, 123, 1275-1279.
[18] L. Ayres, M. R. J. Vos, P. J. H. M. Adams, I. O. Shklyarevskiy, J. C. M. van Hest, Macromolecules 2003, 36, 5967-5973.
[19] L. Adamiak, M. A. Touve, C. L. M. LeGuyader, N. C. Gianneschi, ACS Nano 2017, 11, 9877-9888.
[20] R. M. Conrad, R. H. Grubbs, Angew. Chem. Int. Ed. 2009, 48, 8328-8330.
[21] B. D. Fairbanks, P. A. Gunatillake, L. Meagher, Adv. Drug Deliver. Rev. 2015, 91, 141-152.
[22] M. Cieslewicz, J. J. Tang, J. L. Yu, H. Cao, M. Zavaljevski, K. Motoyama, A. Lieber, E. W. Raines, S. H. Pun, Proc. Natl. Acad. Sci. USA 2013, 110, 15919-15924.
[23] K. E. B. Doncom, L. D. Blackman, D. B. Wright, M. I. Gibson, R. K. O'Reilly, Chem. Soc. Rev. 2017, 46, 4119-4134.
[24] A. Valdebenito, M. V. Encinas, Polym. Int. 2010, 59, 1246-1251.
[25] L. Agemy, D. Friedmann-Morvinski, V. R. Kotamraju, L. Roth, K. N. Sugahara, O. M. Girard, R. F. Mattrey, I. M. Verma, E. Ruoslahti, Proc. Natl. Acad. Sci. USA 2011, 108, 17450-17455.
[26] V. R. Fantin, M. J. Berardi, H. Babbe, M. V. Michelman, C. M. Manning, P. Leder, Cancer Res. 2005, 65, 6891-6900.

### Example 1B: Bioactive Peptide Brush Polymer Via Photoinduced Reversible-Deactivation Radical Polymerization-Supporting Information

### 1. Materials

All amino acids used to prepare peptides by solid phase peptide synthesis (SPPS) were obtained from AAPPTec, Chem-Impex, and NovaBiochem. N,N,N′,N″,N″-Pentamethyldiethylenetriamine (PMDETA, 99%), eosin Y disodium salt (dye content >85%), N-(3-dimethylaminopropyl)-N′-ethylcarbodiimide hydrochloride (EDC·HCl, 99%), 4-(dimethylamino)pyridine (DMAP, 98%)), 6-Fmoc-amino hexanoic acid (97%), and acetate buffer (0.1 M, pH 5) were purchased from Sigma Aldrich and used without purification. 2-(Dodecylthiocarbonothioylthio)-2-methylpropionic acid (DDMAT) was synthesized according to previous literature.¹ 4-((((2-Carboxyethyl)thio) carbonothioyl)thio)-4-cyanopentanoic acid (water-soluble RAFT agent, 95%) and 4-cyano-4-[(dodecylsulfanylthiocarbonyl) sulfanyl] pentanoic acid (CDTPA, 97%) was purchased from Combi-Blocks and used without further purification. Methacryloxyethyl thiocarbamoyl rhodamine B was purchased from Polysciences, Inc. Thermolysin was purchased from Promega. LED strip light (450 nm) was purchased from Amazon.

### 2. Methods

¹H Nuclear Magnetic Resonance (¹H NMR): ¹H NMR spectra were recorded on a Varian Inova spectrometer (500 MHz) in d₆-DMSO or CDCl₃. Chemical shifts are given in ppm downfield from tetramethylsilane TMS.

Analytical High-Performance Liquid Chromatography (HPLC): Analytical HPLC analysis of peptides was performed on a Jupiter 4μ Proteo 90 Å Phenomenex column (150×4.60 mm) using a Hitachi-Elite LaChrom L-2130 pump equipped with UV-Vis detector (Hitachi-Elite LaChrom L2420).

Preparative HPLC: Armen Glider CPC preparatory HPLC was used to purify the peptides. The solvent system consists of (A) 0.1% TFA in water and (B) 0.1% TFA in acetonitrile.

Electrospray Ionization Mass Spectrometry (ESI-MS): ESI-MS spectra of peptides were collected using a Bruker Amazon-SL spectrometer configured with an ESI source in both negative and positive ionization mode.

Transmission Electron Microscope (TEM): Twenty microliters of sample were applied onto a 400 mesh carbon grids (Ted Pella, INC.). The grids were observed on a Hitachi HT 7700 microscope operating at 120 kV. The images were recorded with a slow-scan charge-coupled device (CCD) camera (Veleta 2k×2k).

Gel Permeation Chromatography (GPC): GPC measurements were performed on a set of Phenomenex Phenogel 5μ, 1K-75K, 300×7.80 mm in series with a Phenomex Phenogel 5u, 10K-1000K, 300×7.80 mm columns with HPLC grade solvents as eluents: dimethylformamide (DMF) with 0.05M of LiBr at 60 °C. Detection consisted of a Wyatt Optilab T-rEX refractive index detector operating at 658 nm and a Wyatt DAWN® HELEOS® II light scattering detector operating at 659 nm. Absolute molecular weights and polydispersities were calculated using the Wyatt ASTRA software with dn/dc values determined by assuming 100% mass recovery during GPC analysis.

Dynamic Light Scattering (DLS): DLS analysis was conducted at room temperature on a Zetasizer Nano-ZS (Malvern). The laser for DLS was at a wavelength of 633 nm.

Circular Dichroism Spectrophotometer (CD): CD spectra were measured using a Jasco J-815 spectrometer and each sample was measured from 190 to 260 nm with a slit width of 1 nm, scanning at 1 nm intervals with a 1s integration time. Measurements were taken 3x at 25 °C and then averaged to give the spectra. Notably, the peptide and polymer were dissolved in DIW to a concentration of 100 μM (with respect to peptide concentration).

Atomic Force Microscope (AFM): Samples were prepared by pipetting 50 μl of 10× dilution in water onto 1 cm² freshly cleaved mica and incubated at room temperature for 1 minute before blot drying by holding the edge of the mica onto lint free tissue. AFM images were acquired using a Bruker Dimension FastScan AFM using Fastscan A tips and analyzed with Nanoscope V1.9 software. Images were acquired with a scan rate of 3.6 Hz at 512 pixels by 512 pixels resolution. Images were plane flattened in XY simultaneously and then flattened using a 0 nm threshold.

Confocal Laser Scanning Microscopy (CLSM): Imaging was accomplished using LEICA SP5 II laser scanning confocal microscope with a 63× oil immersion objective at 1.5× optical zoom. All the images were Z-stack images. Slice thickness was 0.26 μm with a scan size of 1024×1024 pixels and a scan speed of 400 Hz. The cell nuclei (stained with DAPI) was accomplished using a 405 nm laser with a 15% laser power. The cell membrane (stained with Wheat Germ Agglutinin, Alexa Fluor 488 Conjugate) was accomplished using a 488 nm laser with a 12% laser power. Cell imaging for Rhodamine fluorescence was accomplished using a 543 nm laser with an 8% laser power.

Flow Cytometry: The cell uptake study was analyzed via flow cytometry using a BD FacsAria Ilu 4-Laser flow cytometer (Becton Dickinson Inc., USA). Mean fluorescence intensity and PE-A-histogram data was prepared for presentation using FlowJo v10.

### 3. Experimental

### 3.1 Preparation of Peptide Monomers Via Solid-Phase Peptide Synthesis (SPPS)

Peptides were synthesized on Rink resin (0.67 mmol/g) using standard FMOC SPPS procedures on an AAPPTec Focus XC automated synthesizer. A typical SPPS procedure included deprotection of the N-terminal Fmoc group with 20% 4-methyl-piperidine in DMF (1×20 min, followed by 1×5 min), and 30 min amide couplings (twice) using 3.0 equiv. of the Fmoc-protected amino acid, 2.9 equiv. of HBTU and 6 equiv. of DIPEA. After that, peptide monomers were prepared by amide coupling to Fmoc-6-aminohexanoic acid, followed by Fmoc deprotection and final amidation with acrylic acid (3 equiv.) in the presence of HBTU (2.9 equiv.), and DIPEA (6 equiv.).

### 3.2 Photo-Polymerization in DMSO

In a typical organic phase photo-induced polymerization (P1), peptide (GPLGLAGG) (SEQ ID NO:5) acrylamide monomer (30 mg, 50 equiv.) and DMA (3.7 mg, 50 equiv.) were dissolved in 150 μL of DMSO. Then 10 μL (1.0 equiv.) of DDMAT stock solution (2.7 mg in 100 μL of DMSO) was added into the reaction mixture. Following that, 10 μL (0.05 equiv.) of eosin Y disodium salt stock solution (2.6 mg in 1 mL of DMSO) and PMDETA (0.13 mg, 1.0 equiv.) were added. The solution was degassed by N₂ flow for 30 min and then placed into the photo-reactor (450 nm, 2.8 mW/cm²) for 24 h. After polymerization, the polymer product was purified by dialysis into DIW, followed by lyophilization.

### 3.3 Photo-Polymerization in Aqueous Solution

In a typical aqueous photo-induced polymerization (P14), KLA peptide (KLAKLAKKLAKLAK) (SEQ ID NO:3) acrylamide monomer (30 mg, 25 equiv.) and DMA (1.8 mg, 25 equiv.) were dissolved in 150 μL of acetate buffer (0.1 M, pH 5). Then 10 μL (1.0 equiv.) of water-soluble RAFT agent stock solution (2.2 mg in 100 μL of acetate buffer) was added into the reaction mixture. Following that, 10 μL (0.05 equiv.) of eosin Y disodium salt stock solution (2.5 mg in 1 mL of acetate buffer) and PMDETA (0.12 mg, 1.0 equiv.) were added. The solution was degassed by N₂ flow for 30 min and then placed into the photo-reactor (450 nm, 2.8 mW/cm²) for 24 h. After polymerization, the polymer product was purified by dialysis into DIW, followed by lyophilization.

### 3.4 Preparation of Rhodamine-Labeled Polymers

In all the cases of preparing rhodamine-labeled polymers, one equiv. of rhodamine B to RAFT agent was used, ensuring that on average one dye was attached to each polymer chain. In a typical polymerization (rhodamine-labeled P14), KLA peptide (KLAKLAKKLAKLAK) (SEQ ID NO:3) acrylamide monomer (30 mg, 25 equiv.), DMA (1.8 mg, 25 equiv.), and methacryloxyethyl thiocarbamoyl rhodamine B (0.48 mg, 1.0 equiv.) were dissolved in 150 μL of acetate buffer (0.1 M, pH 5). Then 10 μL (1.0 equiv.) of water-soluble RAFT agent stock solution (2.2 mg in 100 μL of acetate buffer) was added into the reaction mixture. Following that, 10 μL (0.05 equiv.) of eosin Y disodium salt stock solution (2.5 mg in 1 mL of acetate buffer) and PMDETA (0.12 mg, 1.0 equiv.) were added. The solution was degassed by N₂ flow for 30 min and then placed into the photo-reactor (450 nm, 2.8 mW/cm²) for 24 h. After polymerization, the polymer product was purified by dialysis into DIW, followed by lyophilization.

3.5 N-Acetylation of poly(KLAAm-co-DMA)

In a typical procedure, poly(KLAAm-co-DMA) (1.0 equiv. with respect to free amines) was dissolved in DMF and then treated with 50 equiv. of acetic acid in the presence of 50 equiv. of EDC·HCl and 5 equiv. of 4-DMAP. The reaction mixture was stirred for 12 hours, followed by dialysis into DIW wand lyophilization.

### 3.6 Thermolysin-Induced Cleavage Experiments

For enzyme-triggered cleavage experiments, the molar ratio of thermolysin to peptide was set to 1:300. Moreover, the temperature was set to 55 °C to achieve the optimal activity of thermolysin. For example, poly(PepAm₂₁-CO-DMA₂₄) (P4, 1 mg, 0.87 μmol with respect to peptides, 300 equiv.) was dissolved in 1 ml of DPBS solution. Then thermolysin (0.1 mg, 2.9 nmol, 1 equiv.) was added into the polymer solution which was stirred in a preheated oil bath at 55 °C. In the case of control experiments which involved using deactivated thermolysin, EDTA (100 equiv. to thermolysin) was utilized to capture the zinc and calcium ions, resulting in denaturing of thermolysin.

### 3.7 Cell Culture

Hela cells were purchased from ATCC. Cells were cultured at 37 °C under 5% CO₂ in phenol-red containing Dulbecco's Modified Eagle Medium (DMEM; Gibco Life Tech., cat. #11960-044) supplemented with 10% fetal bovine serum (Omega Scientific, cat. #11140-050), sodium pyruvate (Gibco Life Tech., cat. #35050-061), L-glutamine (Gibco Life Tech., cat. #35050-061), and the antibiotics penicillin/streptomycin (Corning Cellgro, cat. #30-002-C₁). Cells were grown in T75 culture flasks and subcultured at ~75-80% confluency.

### 3.8 Cell Viability Assay

The cytotoxicity of materials was assessed using the CellTilter-Blue assay. Hela cells were plated at a density of 5000 cells per well in a 96 well plate 18 hours prior to treatment. Materials were dissolved in DPBS at the desired concentration and added to the wells along with a 10% DMSO positive control. Cells were incubated for 72 hours at 37 °C. Note that the concentration of all the materials is with respect to the peptide concentration to ensure that all peptides and polymers are fairly compared with respect to their therapeutic components. The media was removed and 80 μL of new media without phenol red was added followed by adding 20 μL of CellTilter-Blue reagent. The cells were incubated for 3 hours a t 37 °C. The fluorescence was measured at 560 nm excitation and 590 nm emission wavelength.

### 3.9 Confocal Laser Scanning Microscopy for Uptake in Hela Cells

Hela cells were plated in a 4-chamber 35 mm round glass-bottom dishes at a density of 50,000 per well. Cells were incubated for 24 hours in a 5% CO₂ atmosphere at 37 °C. 500 μL of KLA peptide, Poly(KLAAm₂₅-CO-DMA₇₅), Poly(KLAAm₂₅-CO-DMA₂₅), and Poly(KLAAm₁₀) (0.25 UM with respect to rhodamine for each material) in 10% FBS DMEM media without phenol red were incubated with the cells for 24 hours, respectively. After washing with DPBS to remove the residual peptides and polymers, 500 μL of Wheat Germ Agglutinin (5 μg/mL) conjugated with Alexa Fluor 488 was added to each well, then fixed with a 4% paraformaldehyde solution for 15 min at room temperature. The cells were washed with DPBS and stained by DAPI for 20 min at room temperature.

3.10 Flow Cytometry for Uptake Ability in Hela cells

For cellular uptake measurements, Hela cells were plated in 12-well plates at a density of 1,000,000 per well and incubated for 24 hours in a 5% CO₂ atmosphere at 37 °C. 500 UL of 0.25 μM (with respect to rhodamine) KLA peptide, poly(KLAAm₂₅-CO-DMA₇₅), poly(KLAAm₂₅-CO-DMA₂₅), and poly(KLAAm₁₀) in 10% FBS DMEM media without phenol red were incubated with the cells for 24 hours respectively. After triple washing with DPBS, 500 μL of 0.25% Trypsin-EDTA was added to each well for 10 mi n at 37 °C. Cells were fixed with a 4% paraformaldehyde solution for 15 min at room temperature.

### References Corresponding to Example 1B

1. Xu, J. T.; Shanmugam, S.; Fu, C. K.; Aguey-Zinsou, K. F.; Boyer, C., Selective Photoactivation: From a Single Unit Monomer Insertion Reaction to Controlled Polymer Architectures. J. Am. Chem. Soc. 2016, 138 (9), 3094-3106.

### Example 2A: Proapoptotic Peptide Brush Polymer Nanoparticles via Photoinitiated Polymerization-Induced Self-Assembly

Abstract: Herein we report photoinitiated polymerization-induced self-assembly (photo-PISA) of spherical micelles consisting of proapoptotic peptide-polymer amphiphiles. The one-pot synthetic approach yielded micellar nanoparticles at high concentrations and at scale (150 mg/mL) with tunable peptide loadings up to 48 wt. %. The size of the micellar nanoparticles was tuned by varying the lengths of hydrophobic and hydrophilic building blocks. Importantly, the peptide-functionalized nanoparticles imbued the peptides, such as proapoptotic “KLA” peptides (amino acid sequence: KLAKLAKKLAKLAK) (SEQ ID NO:3), with two key properties otherwise not inherent to the sequence: 1) proteolytic resistance compared to the oligopeptide alone; 2) significantly enhanced cell uptake permeability by multivalent display of KLA peptide brushes. The result was demonstrated improved apoptosis efficiency in HeLa cells. These results highlight the potential of photo-PISA in the large-scale synthesis of functional, proteolytically resistant peptide-polymer conjugates for intracellular delivery.

Introduction: Synthetic peptides are powerful therapeutics and chemical biology tools because of their biocompatibility, straightforward synthesis, predictable metabolism, and high degree of modularity in molecular design.^{[1]} However, these advantages are typically compromised by natural processes prevalent in cells and tissues that have evolved to degrade them.^{[2]} Traditional approaches for protecting active peptides from enzymatic digestion capitalize on chemical modification of the peptide.^{[3]} These approaches include cyclization,^{[3a-b]} lipidation,^{[3c]} conjugation of PEG,^{[3d-e]} introduction of unnatural amino acids,^{[3f]} peptide backbone modification (e.g., N-methylation),^{[3g]} and capping of N- or C-terminus,^{[3h]} among others.^{[3i]} Consequently, modified peptides are rendered inaccessible to, or unrecognizable by the active site of protease. Nevertheless, the bioactivity or function of modified peptides can be reduced as a result of the alteration of chemical identity and connectivity in amino acids. Moreover, penetration of peptides into cells is typically inefficient unless some special care is taken to engage cell surfaces selectively or through the use of cell penetrating sequences.^{[4]}

Beyond modification of the sequence itself, the three-dimensional spatial arrangement of peptides not only can improve the stability of peptides, but also enhance their biological activities such as cell binding and penetration via multivalent effects.^{[5]} Examples include peptide-coated inorganic nanoparticles,^{[6]} peptide-shell polymer nanoparticles,^{[7]} and peptide brush polymers,^{[7c, 8]} all of which display multiple strands of peptides on a scaffold. However, the synthesis of these materials is not scalable, hindering continued development of these systems to larger animal models and clinical translations.^{[7d]} Standard solvent-switch strategies to access self-assembled nanostructures of peptide-polymer amphiphiles are typically conducted in dilute solution (<1 wt. %).^{[7c]}

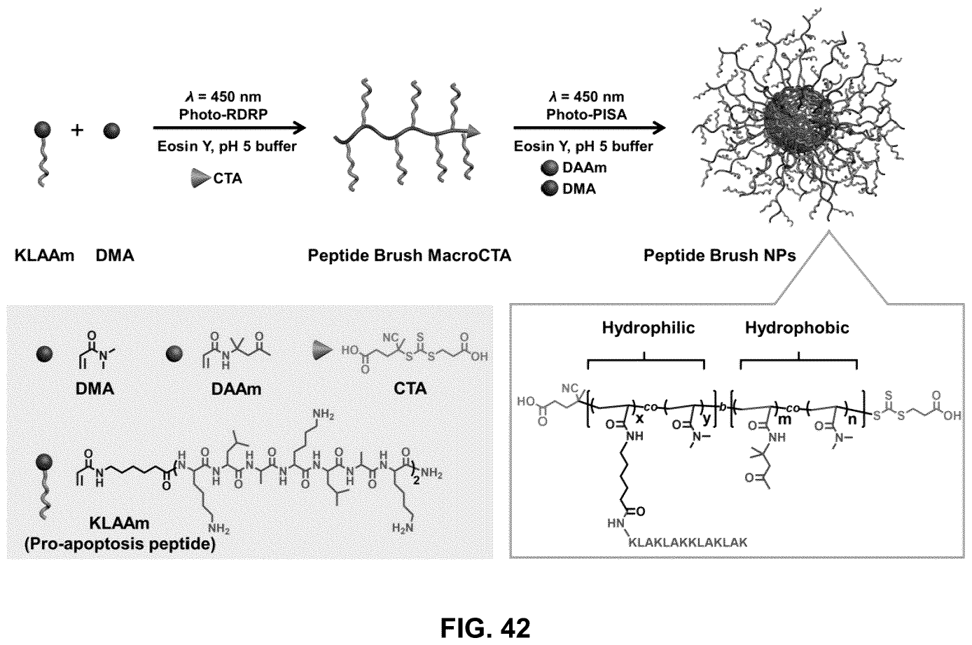

Polymerization-induced self-assembly (PISA) has emerged as a scalable synthetic route to soft nanomaterials at high solids content (up to 50 wt. %).^{[9]} Particularly, photoinitiated PISA (photo-PISA) is promising for the incorporation of biological molecules into nanoparticles, because the reaction can be performed under mild conditions characterized by ambient temperatures, aqueous environments, and metal-free protocols.^{[10]} We reasoned that photo-PISA could be used as a powerful tool for the large-scale synthesis of polymer nanoparticles that exhibit multiple peptides in the hydrophilic shell (FIG. 42). Herein, we present a one-pot photo-PISA approach for the preparation of nanoparticles that carry apoptotic peptides with tunable size (36-105 nm in diameter) and loading of peptides ranging from 20 to 48 wt. %. High concentrations of peptide brush polymer nanoparticles of up to 150 mg/mL were achieved because of the nature of PISA. Importantly, both proteolytical resistance and bioactivity, including cell penetration and apoptotic efficiency, were significantly higher for the peptide brush polymer nanoparticles compared to their linear peptide analogue.

### Results and Discussion

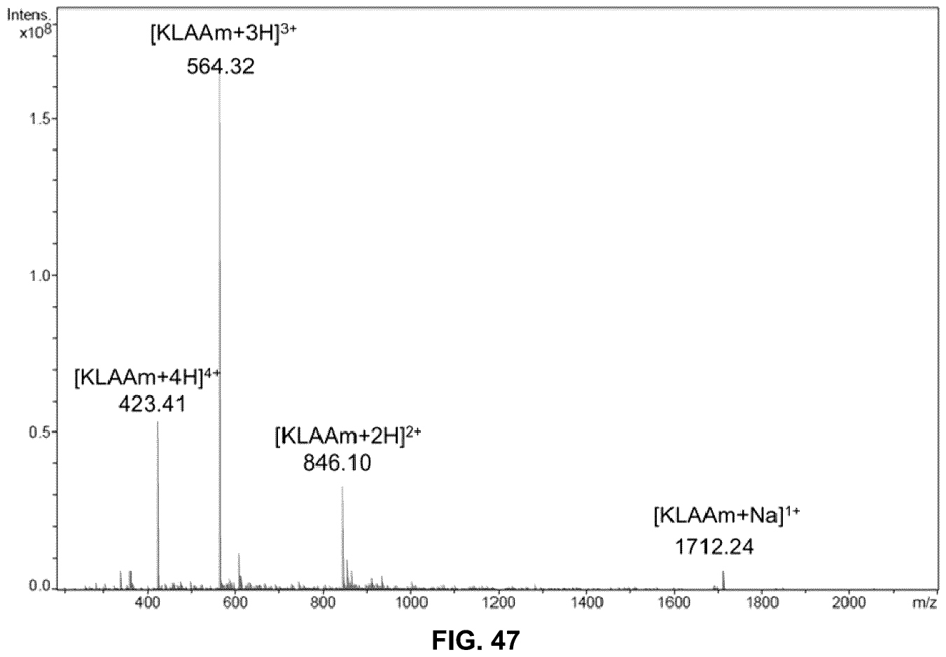

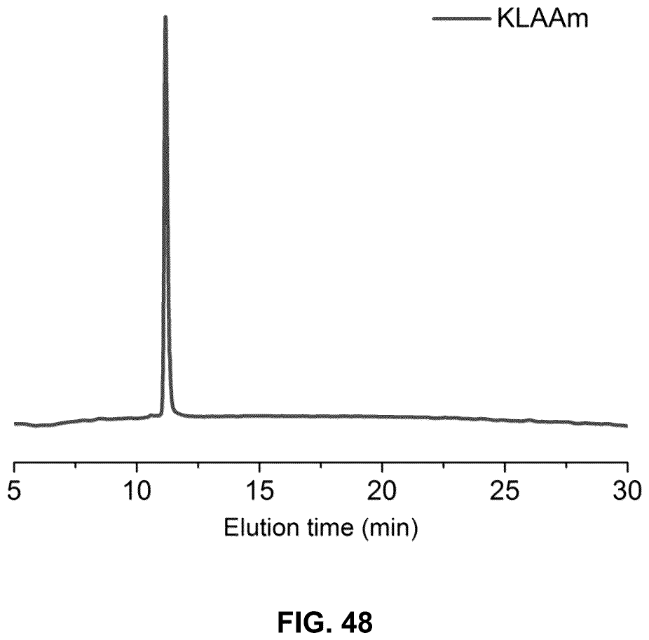

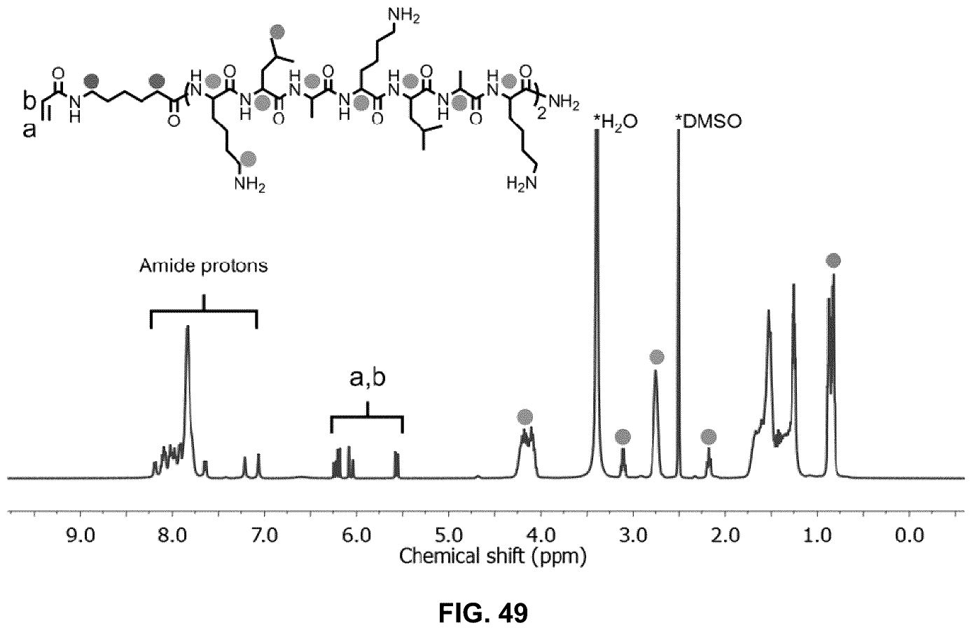

With the goal of preparing peptide brush polymer nanoparticles, we began our exploration by designing a peptide acrylamide monomer that features the amino acid sequence of KLAKLAKKLAKLAK (i.e., KLA peptide acrylamide) (SEQ ID NO:3). This peptide sequence is well known for inducing rapid apoptosis of cancer cells via disruption of the mitochondrial membrane.^{[11]} The monomer was prepared via the Fmoc solid-phase peptide synthesis procedure.^{[7c]} High performance liquid chromatography (HPLC), NMR spectroscopy, and electrospray ionization mass spectrometry (ESI-MS) verified the identity and purity of the monomer (FIGS. 47-49).

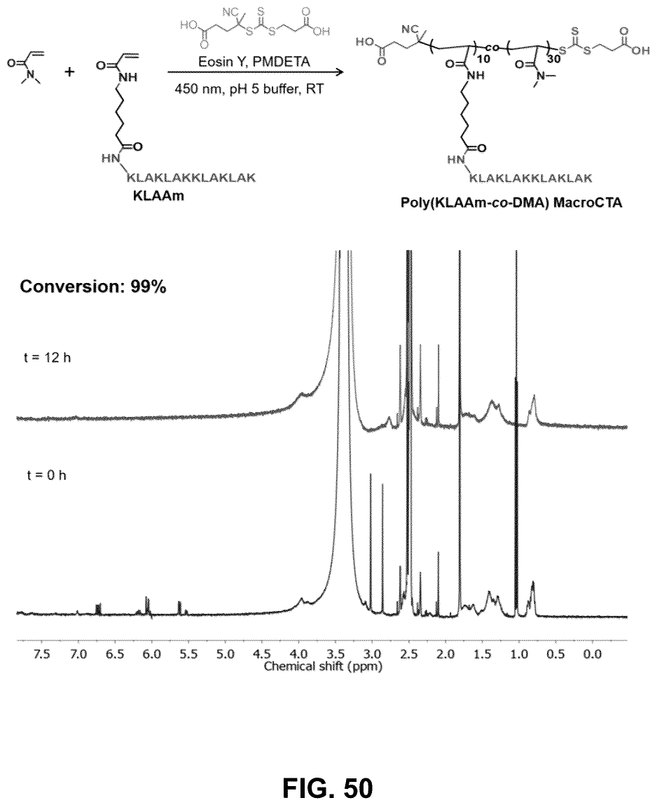

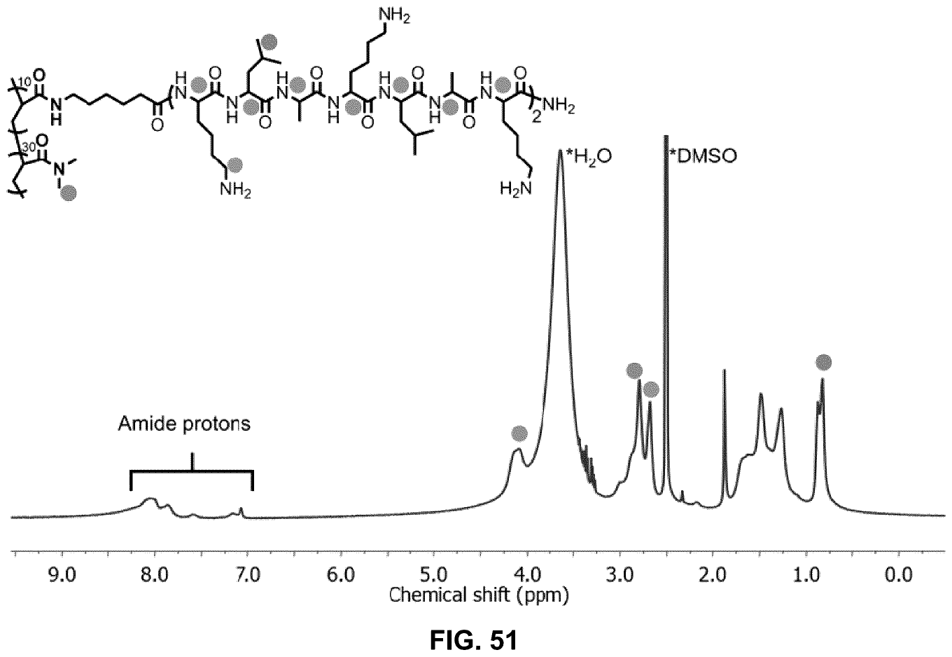

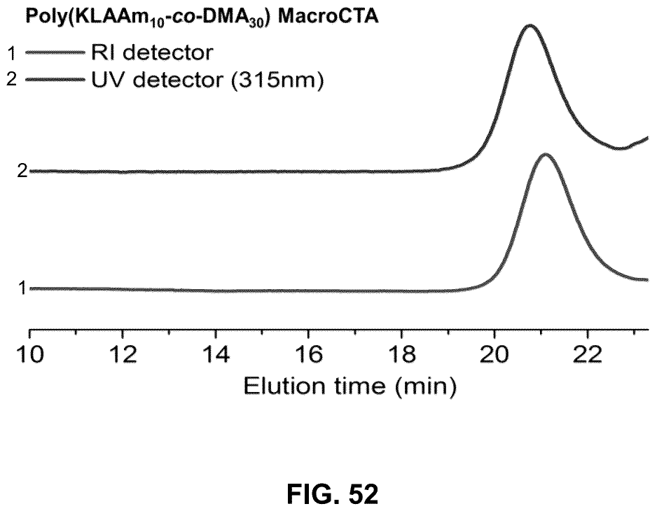

Photoinduced reversible-deactivation radical polymerization (Photo-RDRP)^{[10a,12]} was used to prepare the macromolecular chain transfer agents (macroCTAs) (FIG. 42). To suppress the nucleophilicity of primary amines that could cause aminolysis of the CTA, an acidic buffer (pH 5.0) was used as the solvent for polymerization. Notably, copolymerization of KLA peptide acrylamide monomer (KLAAm) and a comonomer N, N-dimethylacrylamide (DMA) was conducted in the presence of a biocompatible organic photocatalyst (i.e., eosin Y) at room temperature under visible light irradiation (λ=450 nm). The feed ratio of KLAAm and DMA was varied to tune the loading and graft density of peptides along the hydrophilic polymer chain. The monomers were fully consumed after photo-polymerization for 12 h (FIGS. 50-51). Since the photo-polymerization was conducted at room temperature, we postulated that side reactions such as hydrolysis of terminal trithiocarbonate would be minimized, thus leading to high end-group fidelity. Indeed, gel permeation chromatography (GPC) analysis with integrated UV detection confirmed our hypothesis by revealing that the GPC trace of the macroCTA exhibited the characteristic trithiocarbonate absorption at 315 nm (FIG. 52).

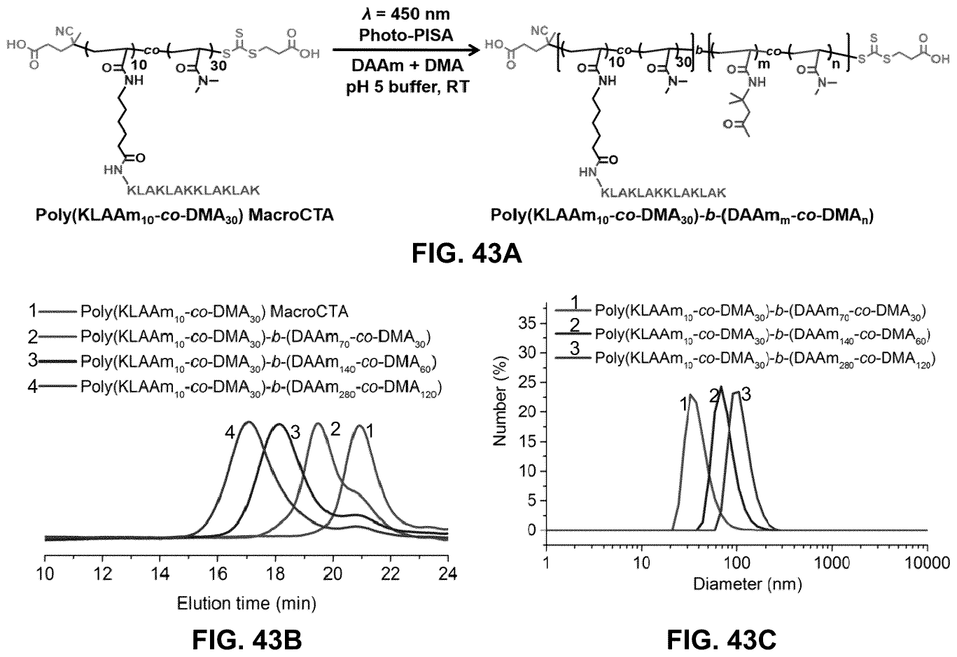

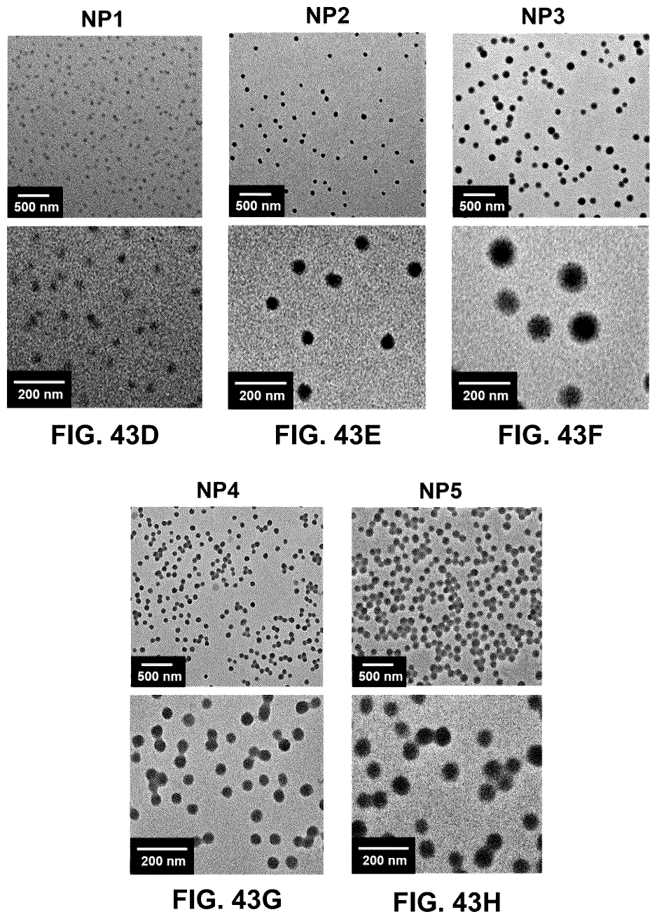

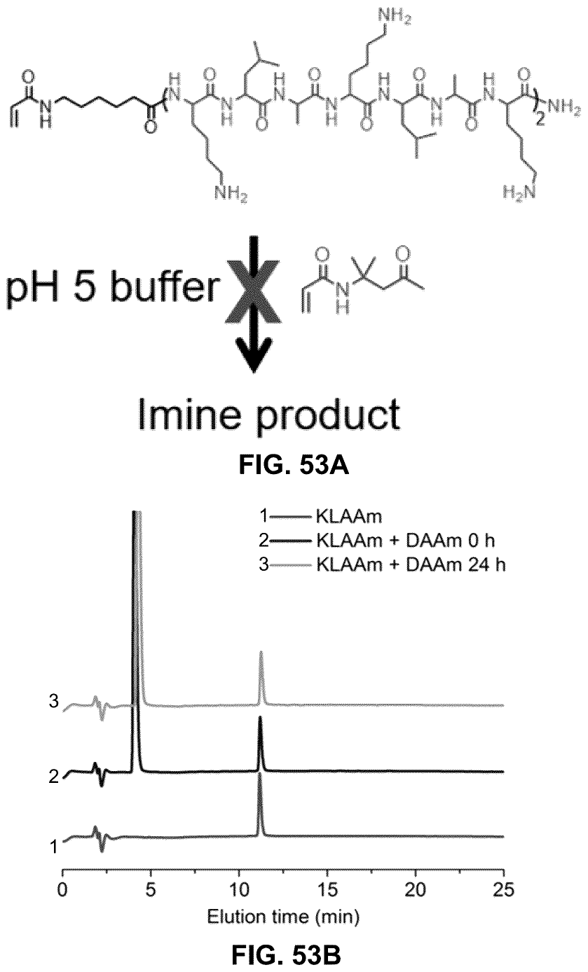

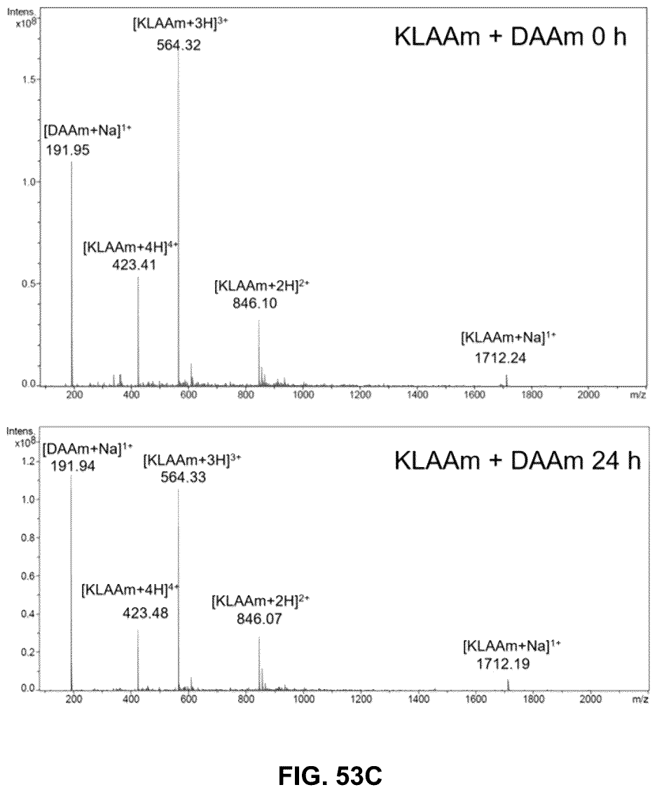

Next, we aimed to perform photo-PISA by chain extension of the macroCTA with a combination of diacetone acrylamide (DAAm) and DMA, which have been shown as readily tunable core-forming monomers in PISA processes (FIGS. 43A-43H).^{[9f,14]} The macroCTA contains primary amines which could potentially form imines with the ketone-containing DAAm. In view of this, we conducted an experiment that involved incubation of KLAAm and DAAm in acidic buffer (pH 5.0). As indicated by HPLC and ESI-MS (FIGS. 53A-53C), no imine products were detected even after 24 h. Therefore, we considered any undesired imine formation during the photo-PISA process would be negligible.

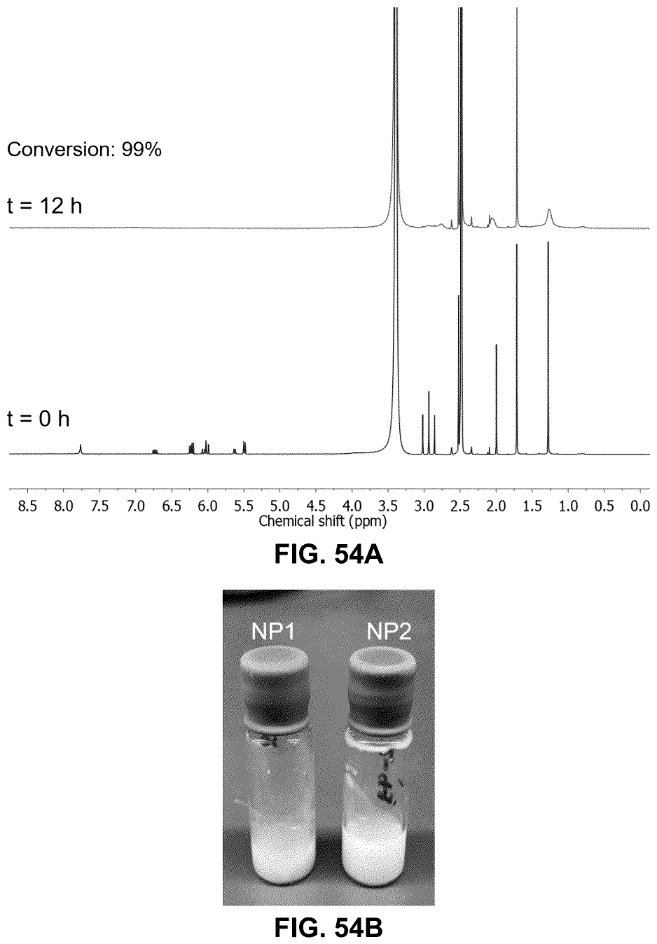

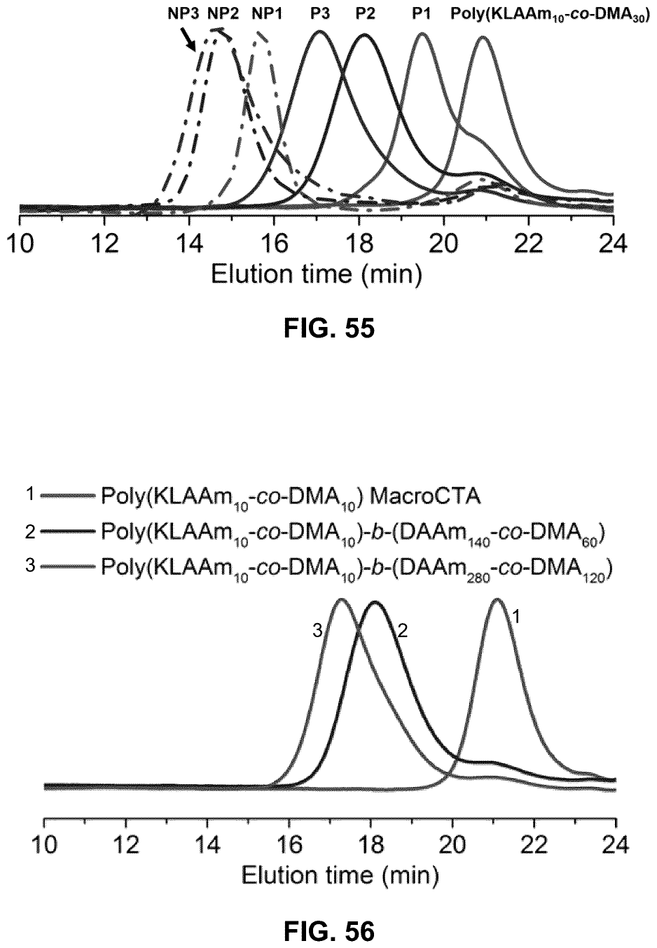

Photo-PISA was performed under the identical condition used in the macroCTA synthesis (i.e., eosin Y and acidic buffer). In light of this, the one-pot synthesis of nanoparticles was achieved without isolating the macroCTA from the buffer solution in which it was synthesized. The efficiency of photo-PISA was revealed via NMR spectroscopy analysis that indicated quantitative conversion of monomers after 12 h (FIGS. 54A-54B). Moreover, GPC traces of block copolymers have clearly shifted to higher molecular weight regions with narrow MW distribution, indicative of successful chain extensions (Table 5, Table 6, FIGS. 2A-2B, and FIGS. 55-56). We note that the GPC signal of residual macroCTA remained in all block copolymers (FIG. 2B). However, the extent of residual macroCTA was significantly decreased as the targeted degree of polymerization (DP) increased. The modularity of peptide brush polymer nanoparticles was examined by tuning variables including compositions in hydrophilic macroCTA and hydrophobic polymer core. Hence, five peptide brush polymer nanoparticles with different sizes and loadings of peptide were achieved (Table 5).

TABLE 5
Peptide brush polymer nanoparticles via photo-PISA at solids content of 15 wt. %.

| | | | | | Peptide | | | | |
|---|---|---|---|---|---|---|---|---|---|
| | Shell-forming | | Core-forming | | Loading^{a} | M_{n, theo}^{b} | M_{n, MALS}^{c} | | Dₕ^{d} |
| Entry | KLAAm | DMA | DAAm | DMA | (wt. %) | (g/mol) | (g/mol) | Ð^{c} | (nm) |
| NP1 | 10 | 30 | 70 | 30 | 48 | 34 980 | 41 700 | 1.29 | 36 |
| NP2 | 10 | 30 | 140 | 60 | 33 | 51 160 | 57 300 | 1.24 | 62 |
| NP3 | 10 | 30 | 280 | 120 | 21 | 80 640 | 98 600 | 1.17 | 105 |
| NP4 | 10 | 10 | 140 | 60 | 38 | 49 180 | 54 900 | 1.03 | 64 |
| NP5 | 10 | 10 | 280 | 120 | 24 | 78 660 | 92 150 | 1.15 | 92 |

^{a}Peptide loading was calculated by feed ratios.
^{b}M_{n, theo} = ΣDP of monomers × MW of monomers + MW of CTA.
^{c}Number-average molecular weights of polymers were determined by GPC-MALS.
^{d}Hydrodynamic diameters of NPs were determined by DLS.

TABLE 6
Characterizations of peptide brush polymer nanoparticles.

| | Unimer | Nanoparticle | Aggregation | | | Surface grafting | |
|---|---|---|---|---|---|---|---|
| | M_{n, MALS} | M_{n, MALS} | number | Rₕ | R_{core} | density | Zeta potential |
| ID | (kg/mol)^{a} | (kg/mol)^{a} | (N_{agg})^{b} | (nm)^{c} | (nm)^{d} | (Chains per nm²)^{e} | (mV) |
| NP1 | 41.7 | 4874 | 117 | 18 | 13 | 0.055 | 47 |
| NP2 | 57.3 | 49040 | 856 | 31 | 28 | 0.087 | 56 |
| NP3 | 98.6 | 152000 | 1542 | 52 | 46 | 0.058 | 48 |

^{a}Molecular weights of unimers and core-crosslinked nanoparticles were determined by GPC-MALS.
^{b}N_{agg} = M_{n, MALS} of NP/M_{n, MALS} of unimer.
^{c}Hydrodynamic radius (Rₕ) of nanoparticles was determined by DLS.
^{d}R_{core} of nanoparticles was estimated by TEM analysis.
^{e}Surface grafting density = N_{agg}/4π R_{core}²

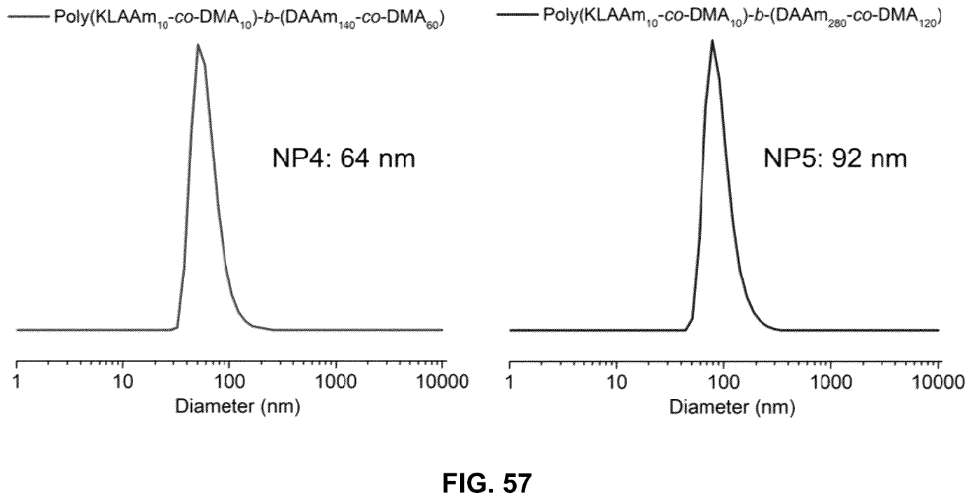

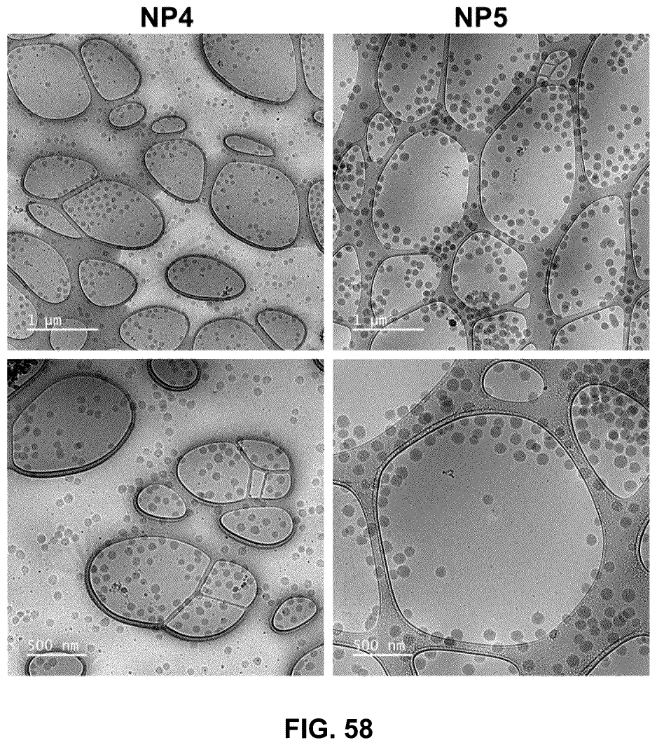

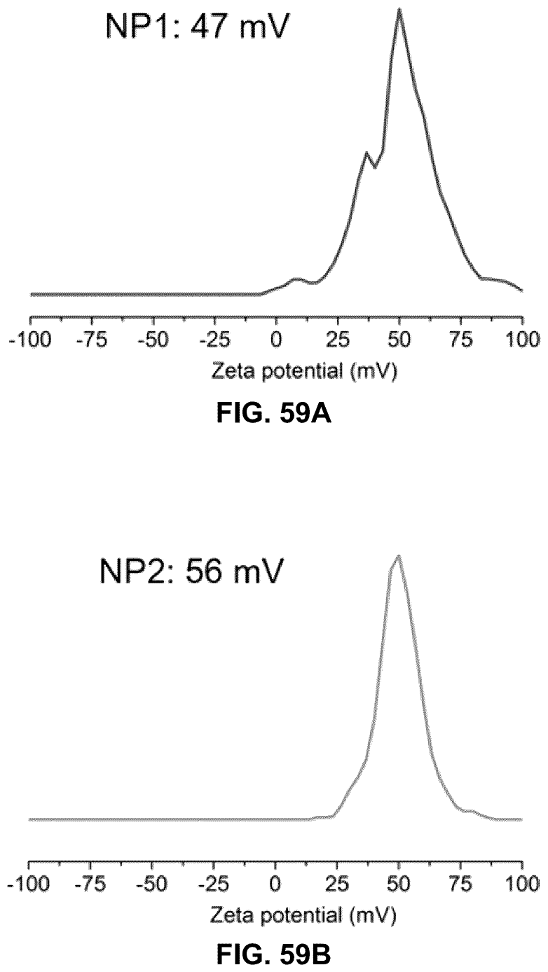

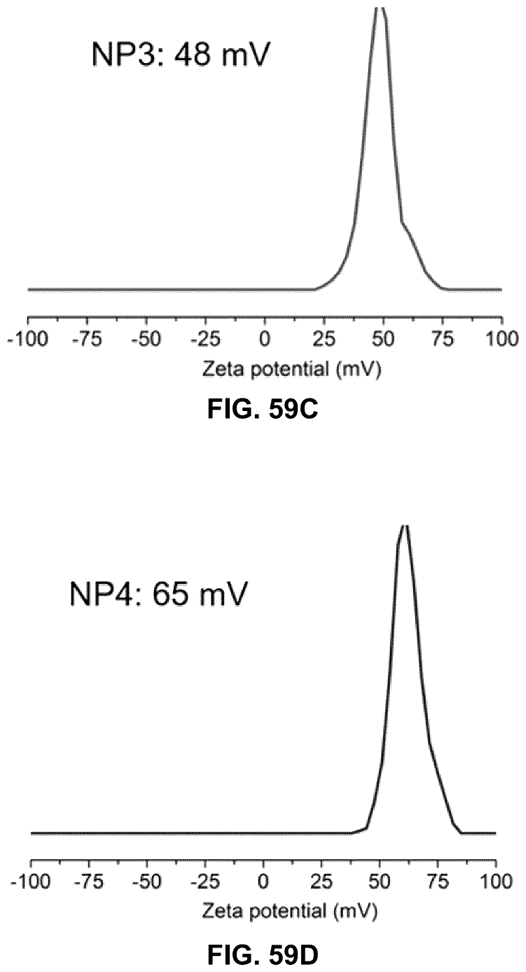

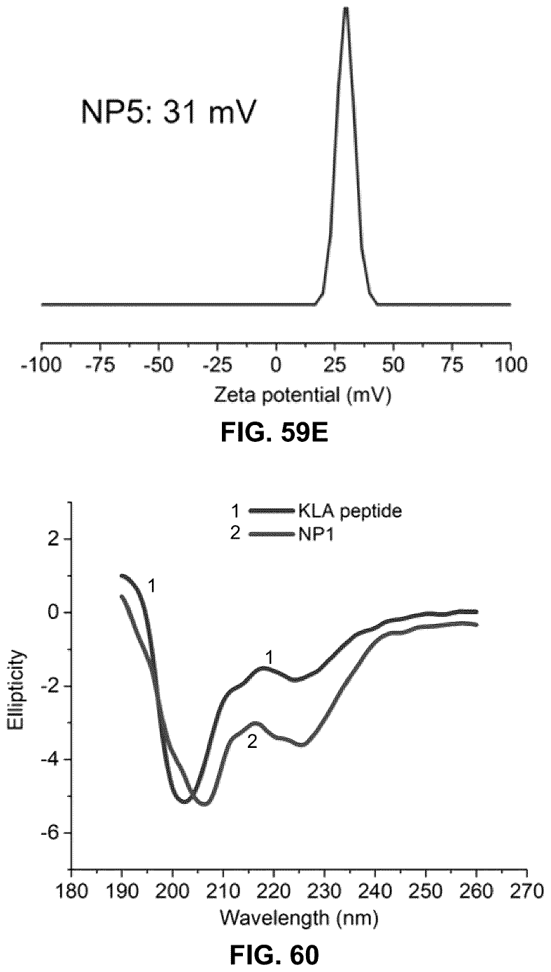

The hydrodynamic diameters of peptide brush polymer nanoparticles were determined by dynamic light scattering (DLS), which suggested a trend of increasing size as the length of the hydrophobic chain increased (FIG. 43C and FIG. 57). Transmission electron microscopy (TEM) further revealed the shape and dry-state size of peptide brush polymer nanoparticles (FIGS. 43D-43H). According to the TEM micrographs, all the peptide brush polymer nanoparticles exhibit spherical morphologies and uniform size distributions. In addition, the nanoparticle size increased as the degree of polymerization of the hydrophobic core increased. This is in a good agreement with DLS and cryogenic transmission electron microscopy analysis (FIG. 58). Notably, only spherical morphology of nanoparticles was observed even at high DPs of core-forming monomers. This can be attributed to the high surface curvature which stems from the positively charged peptide brush shell. The net charge of the nanoparticles was further assessed (FIGS. 59A-59E). Zeta potentials of those nanoparticles were positive, ranging from 31 mV to 65 mV because KLA peptide brush polymer nanoparticles have an abundant number of free amines. Furthermore, the secondary structures of KLA peptide and nanoparticles were evaluated by circular dichroism spectroscopy, which exhibited consistent patterns with a mixture of α-helix and random coil conformations (FIG. 60).

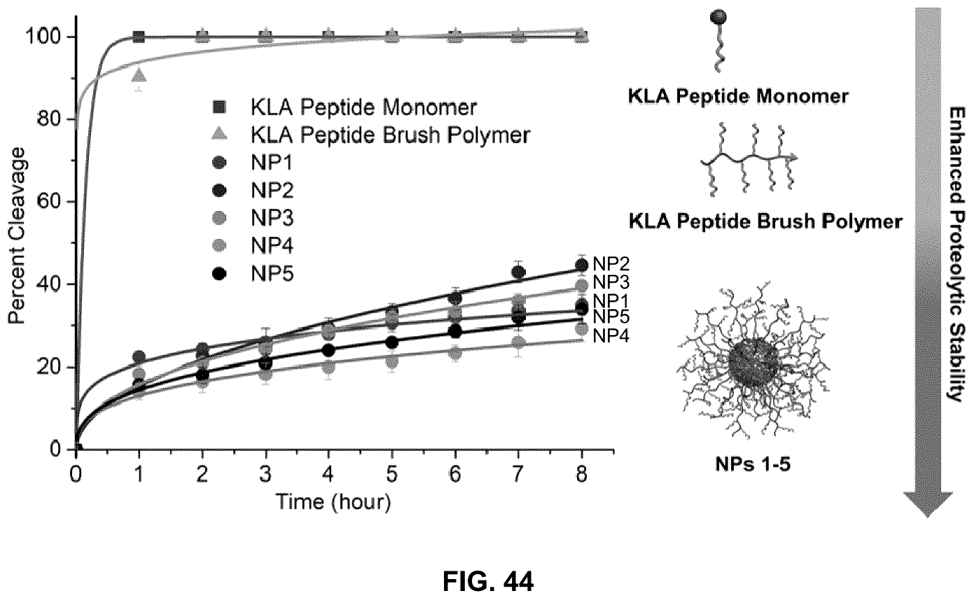

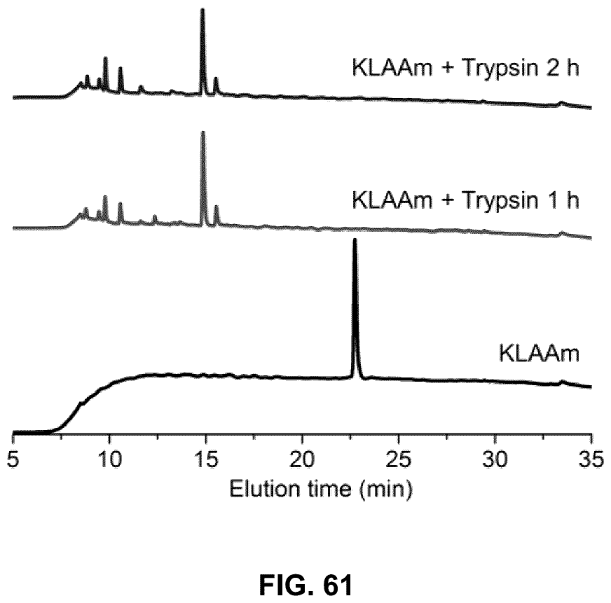

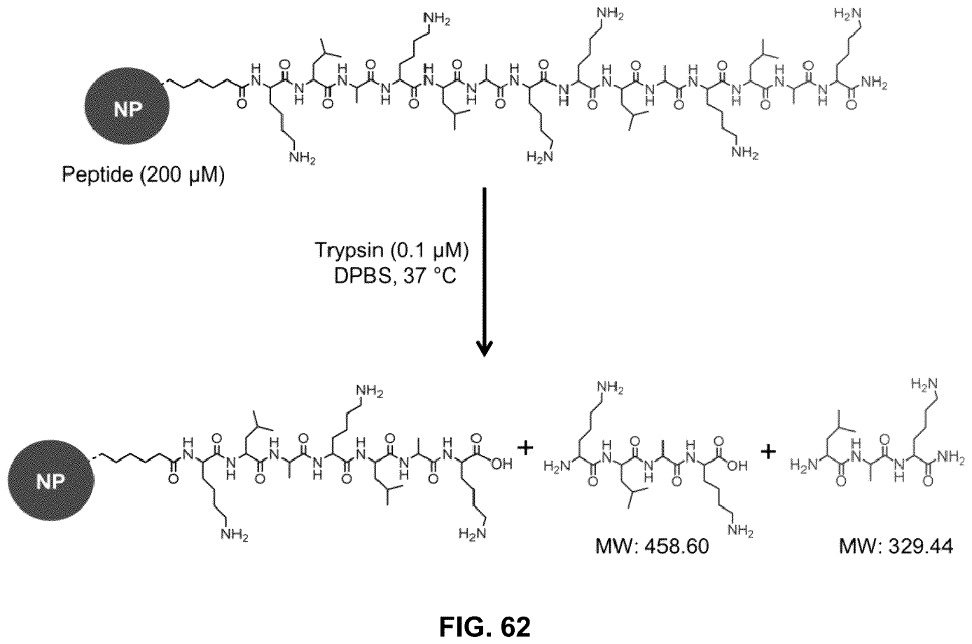

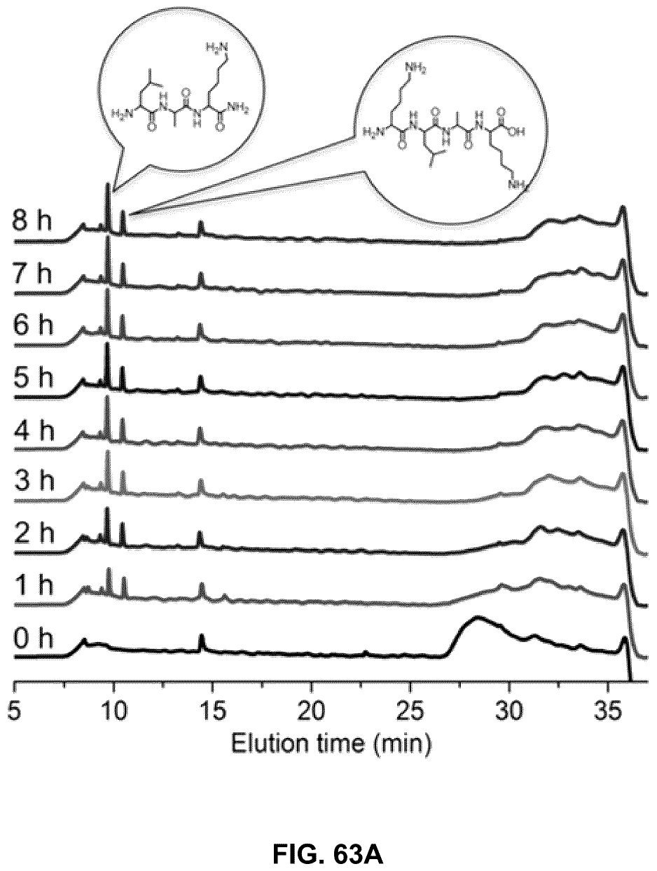

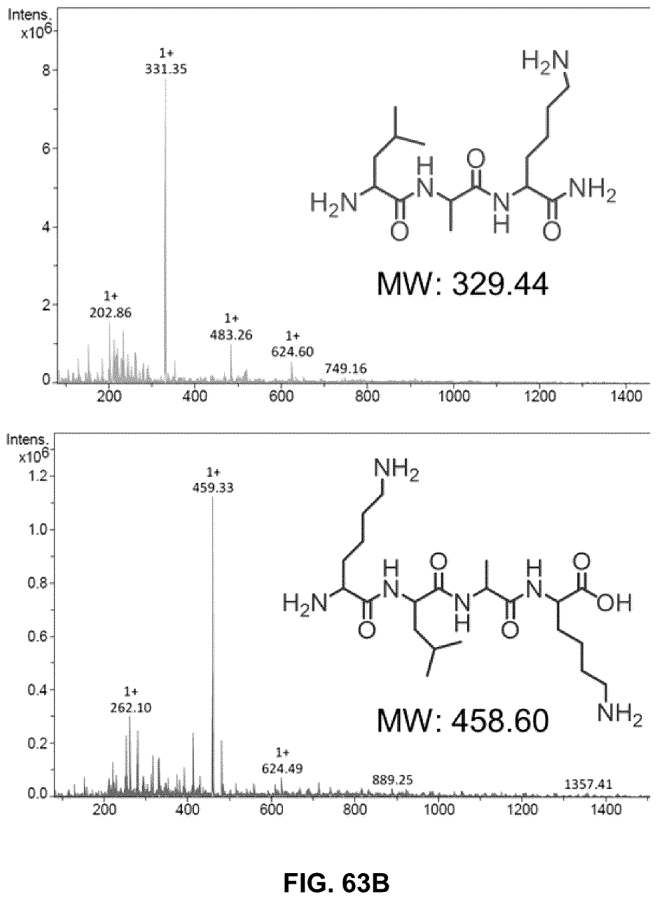

Despite the promise of proapoptotic KLA peptide as anti-cancer therapeutics, the anti-cancer efficacy of free KLA peptide is significantly impeded by its low proteolytic stability as well as poor cell uptake efficiency. Since peptide brush polymer nanoparticles possess a high-density display of KLA peptides on the nanoparticle surface, we reasoned that the stability of the peptides would potentially be enhanced due to steric hindrance limiting access of the peptides to the active sites of proteases.^{[7c,17]} In view of this, we examined the proteolytic resistance of KLA-containing materials of three kinds; 1) peptide, 2) peptide brush polymer, and 3) peptide brush polymer nanoparticles. For this test, trypsin was used as a potent proteinase typically found in the digestive system and freely capable of cleaving the KLA peptide (FIG. 44). The concentration of trypsin was set to 0.1 μM, notably much higher than the level of trypsin in serum. According to HPLC analysis, the KLA peptide underwent fast degradation, reaching 100% cleavage within 1 h (FIG. 61). Similarly, rapid degradation was observed for poly(KLAAm₁₀-co-DMA₁₀), for which more than 90% of the side-chain peptides were cleaved within 1 h. On the other hand, in the case of KLA brush polymer nanoparticles, more than 70% of KLA peptide survived during the first hour of cleavage (FIG. 44, and FIGS. 62-63). This result confirmed that a high-density array of peptides on nanoparticle surface can endow the dangling peptides with enhanced proteolytic stability.

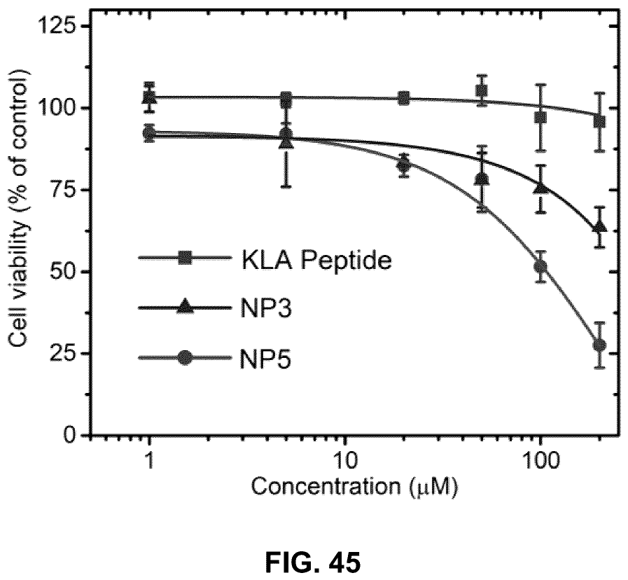

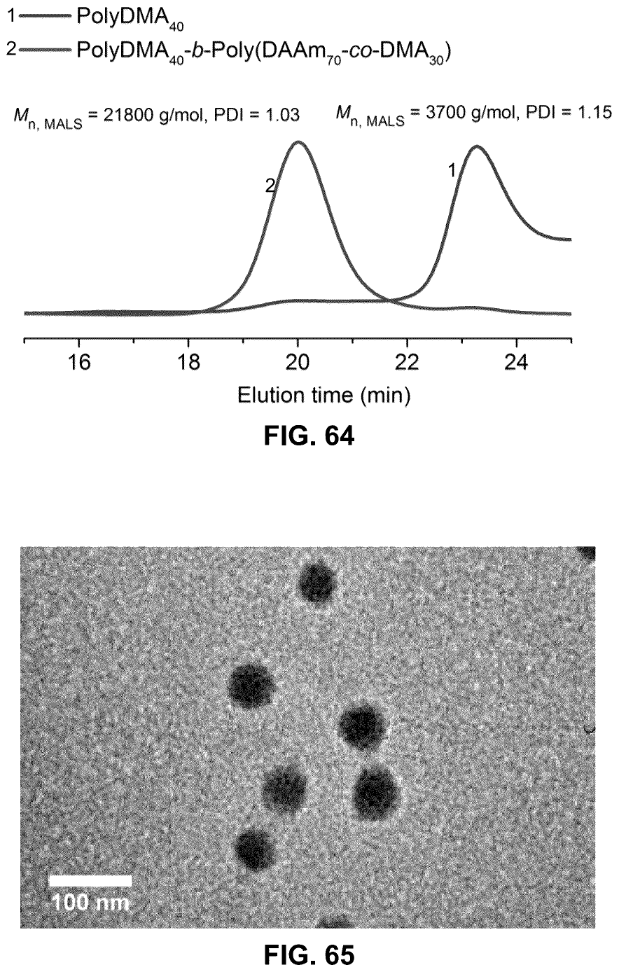

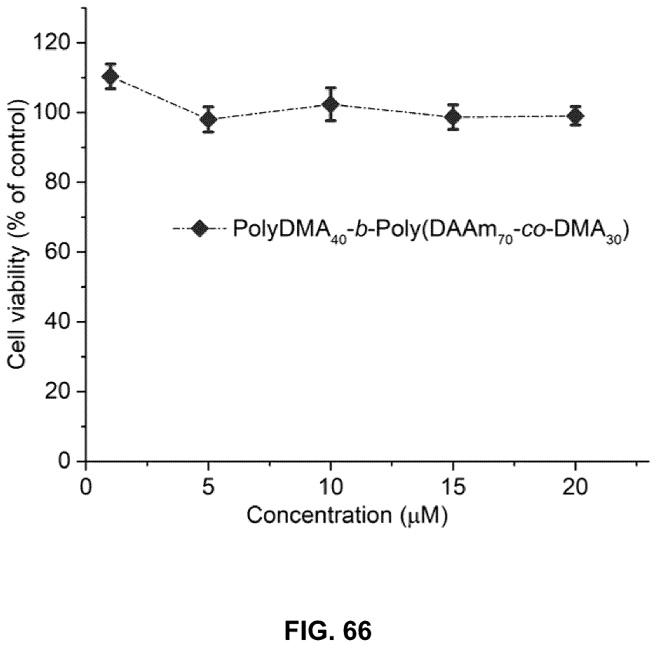

The cytotoxicity of KLA peptide brush nanoparticles and KLA peptide was examined in vitro with human cervical cancer (HeLa) cells (FIG. 45). Two nanoparticles including NP3 and NP5 were chosen to compare in the cell studies because of their similar sizes but different grafting density of peptides on the hydrophilic chain. According to the cell viability assay, NP3 and NP5 demonstrated dose-dependent cytotoxicity in Hela cells, whereas no toxicity was observed for free KLA peptide even at a high concentration of 200 μM. To unequivocally credit the toxicity of KLA brush nanoparticles to the proapoptotic peptides on the nanoparticles, the cytotoxicity of a spherical polymer nanoparticle without carrying the peptides (i.e., polyDMA₄₀-b-poly(DAAm₇₀-co-DMA₃₀)) was further evaluated (FIGS. 64-66). Cell viability assay revealed a high viability (>90%) of Hela cells in the presence of peptide-free polymer micelles under the investigated concentrations, confirming the cytocompatibility of the polymer nanocarrier.

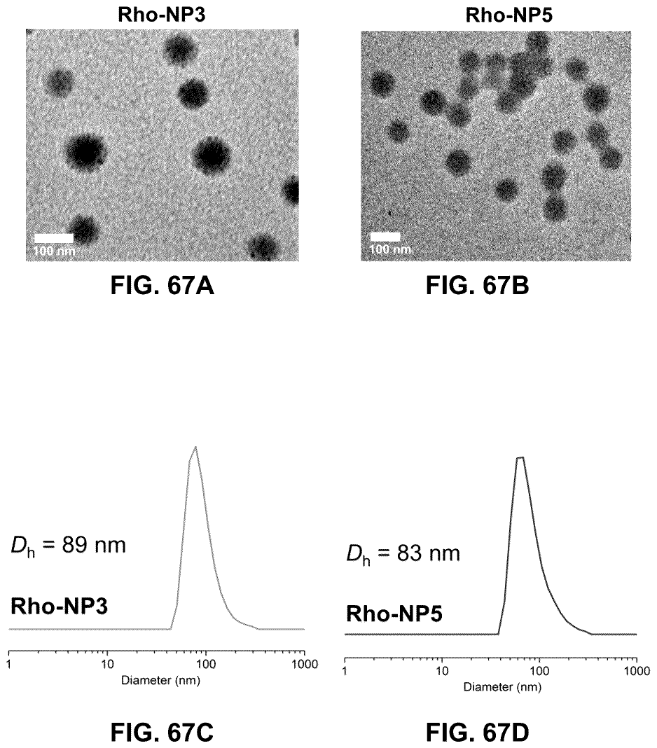

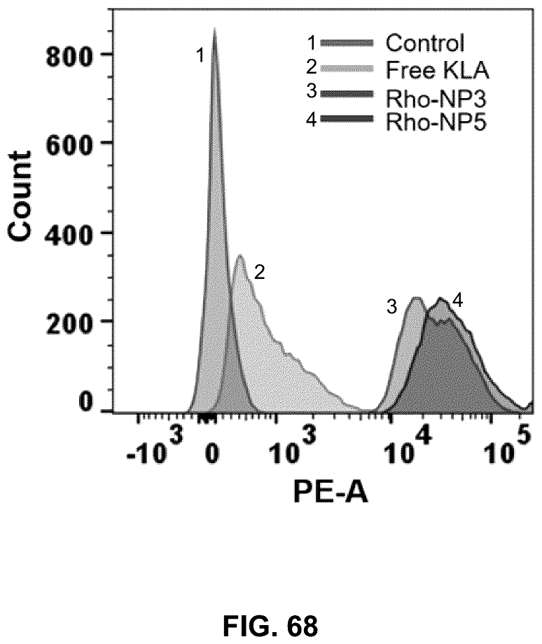

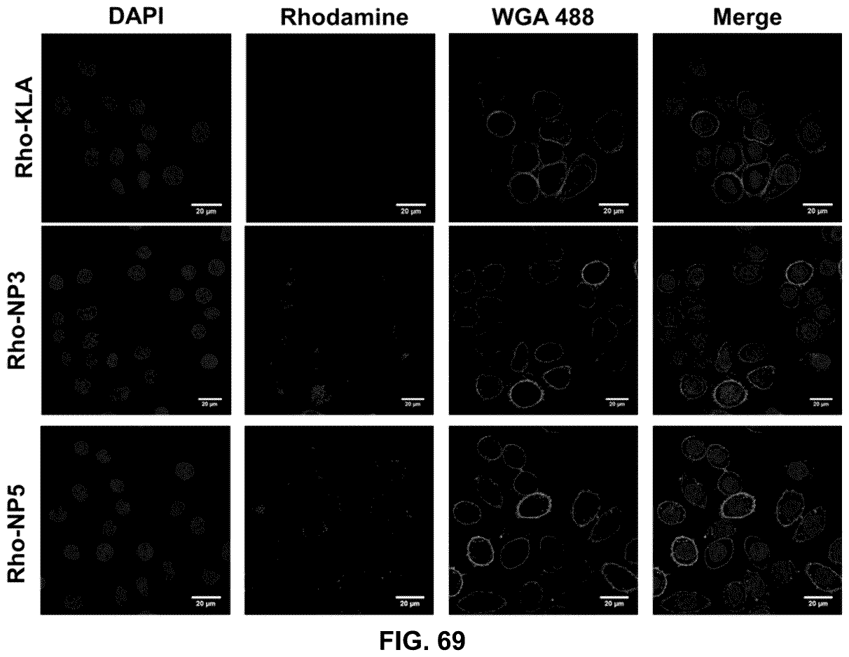

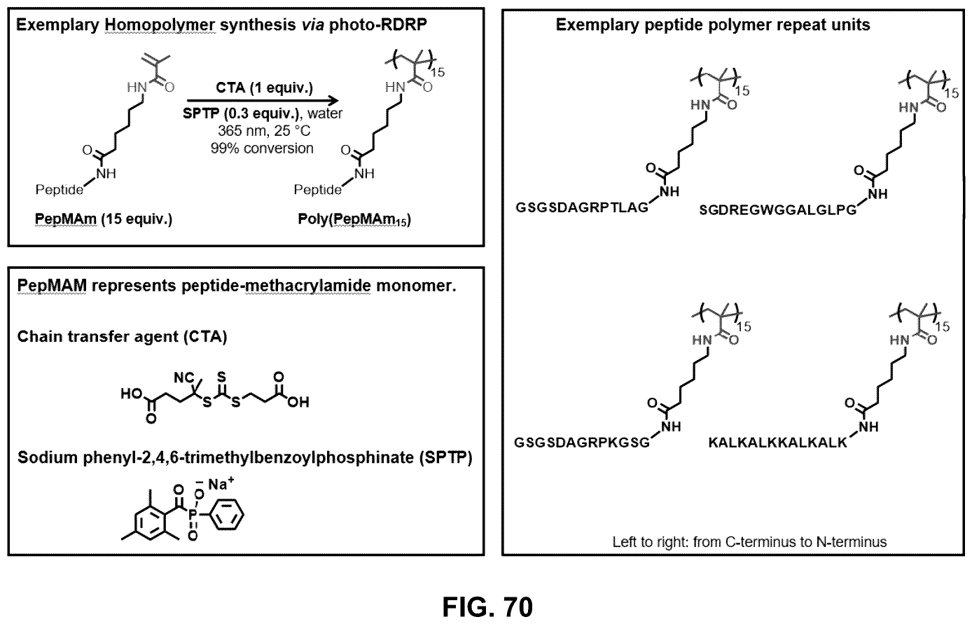

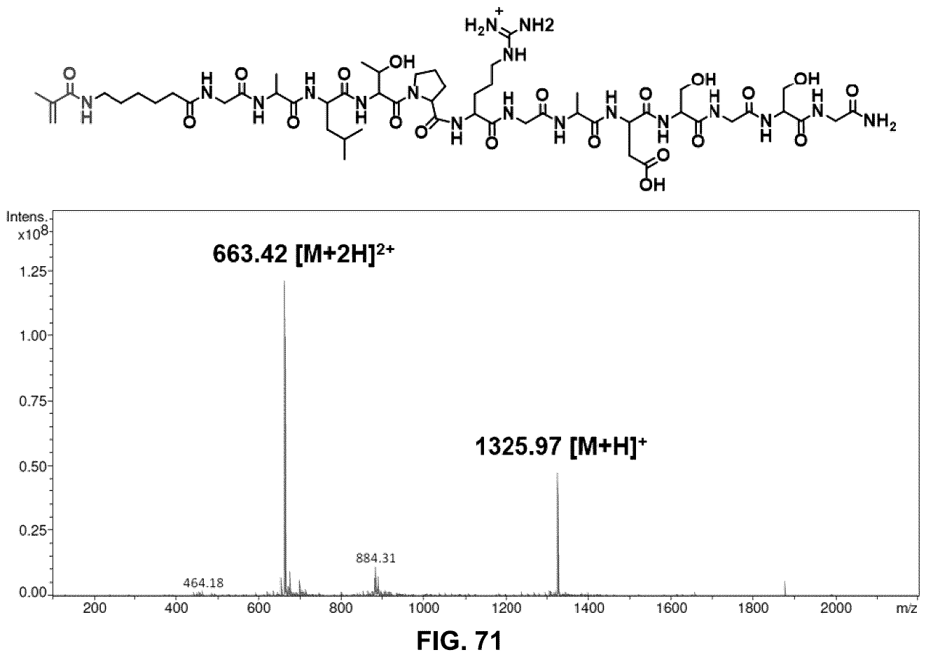

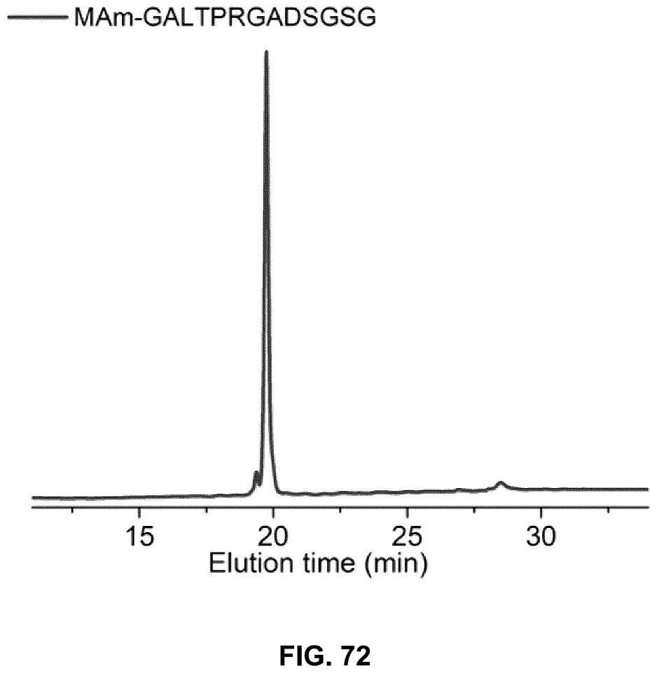

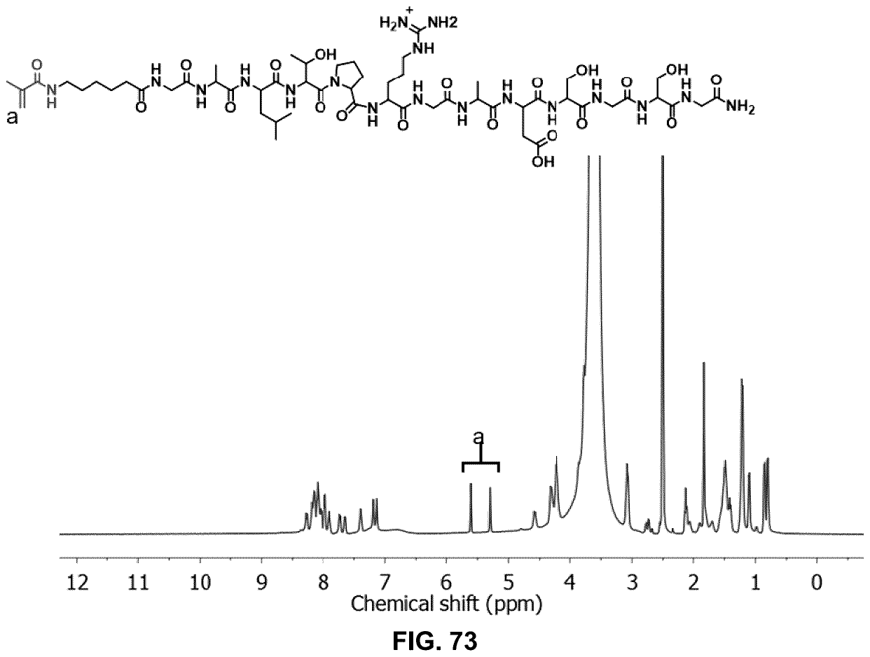

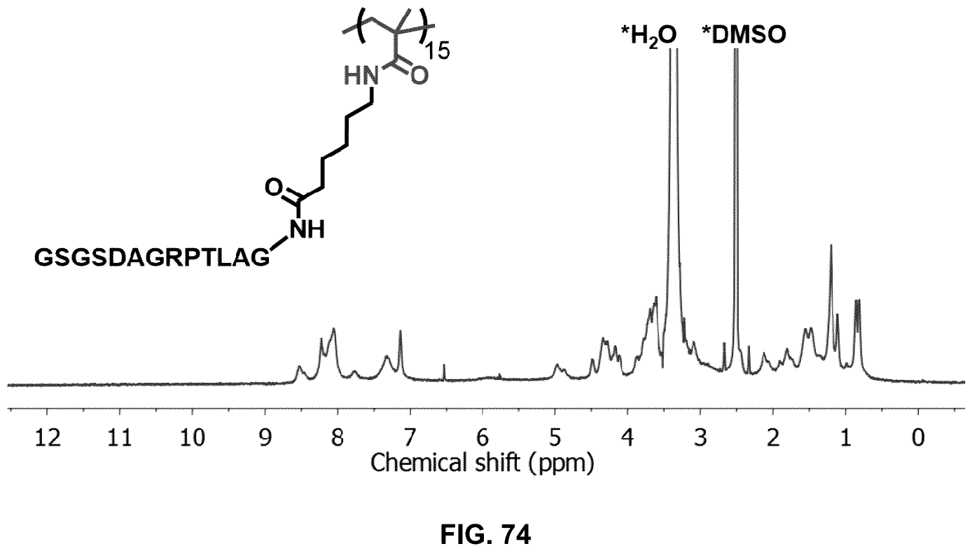

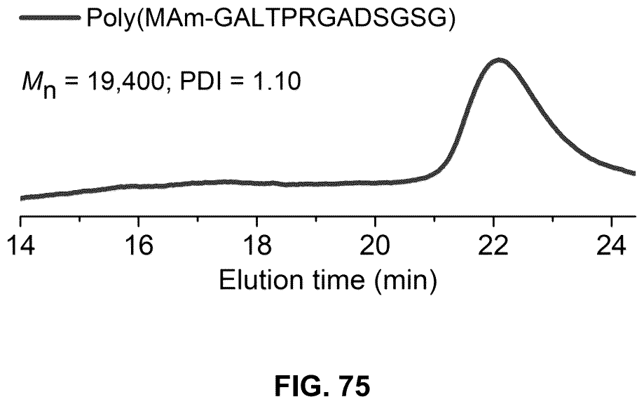

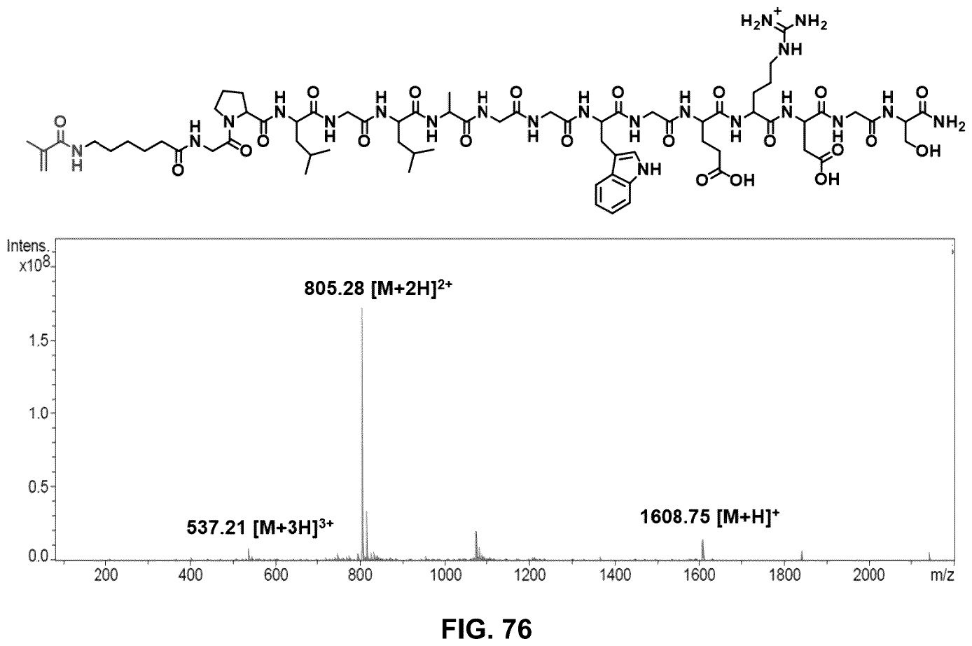

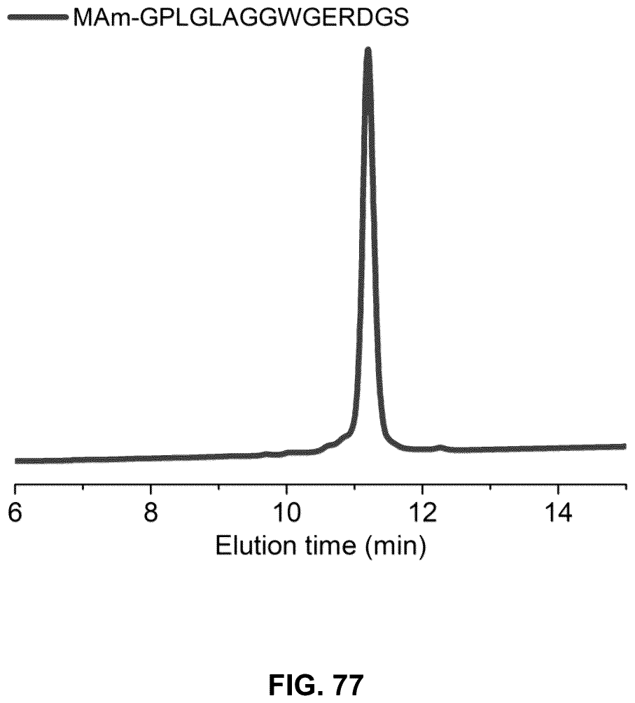

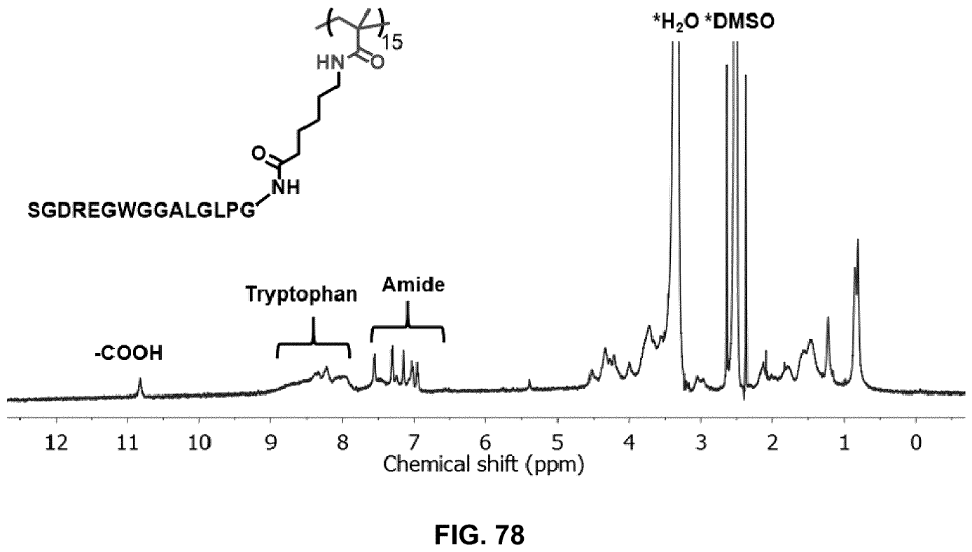

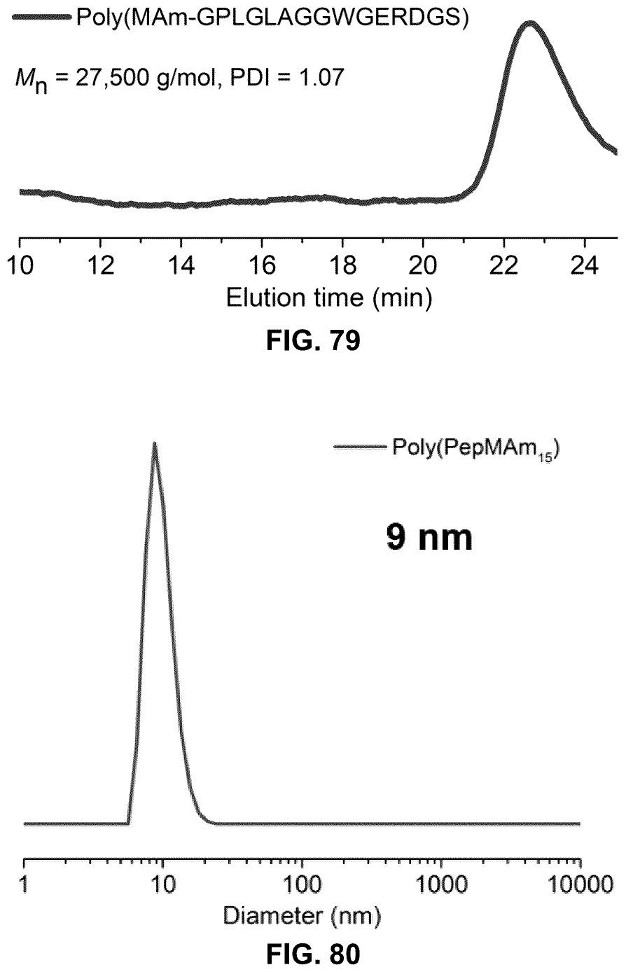

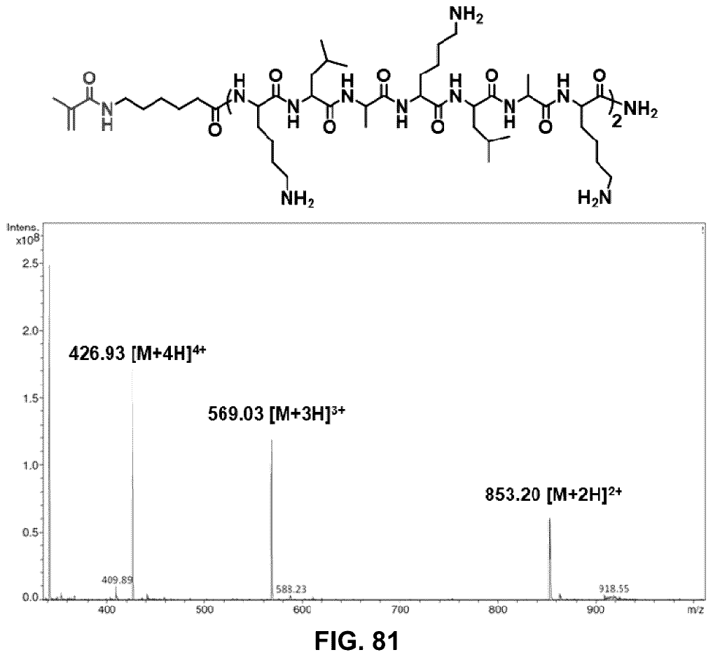

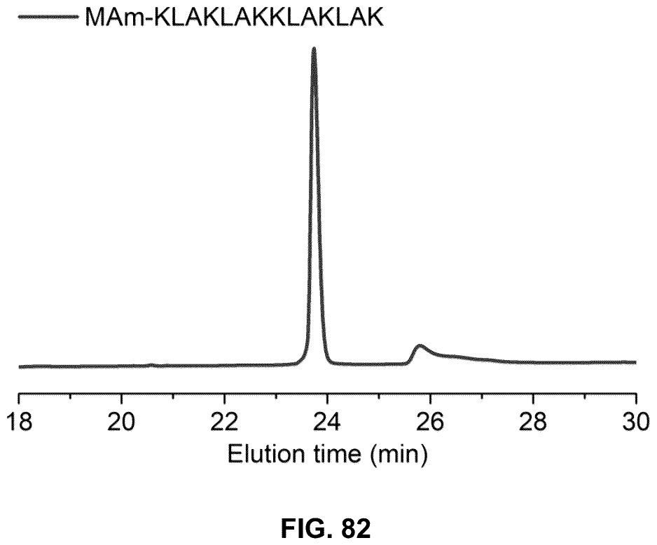

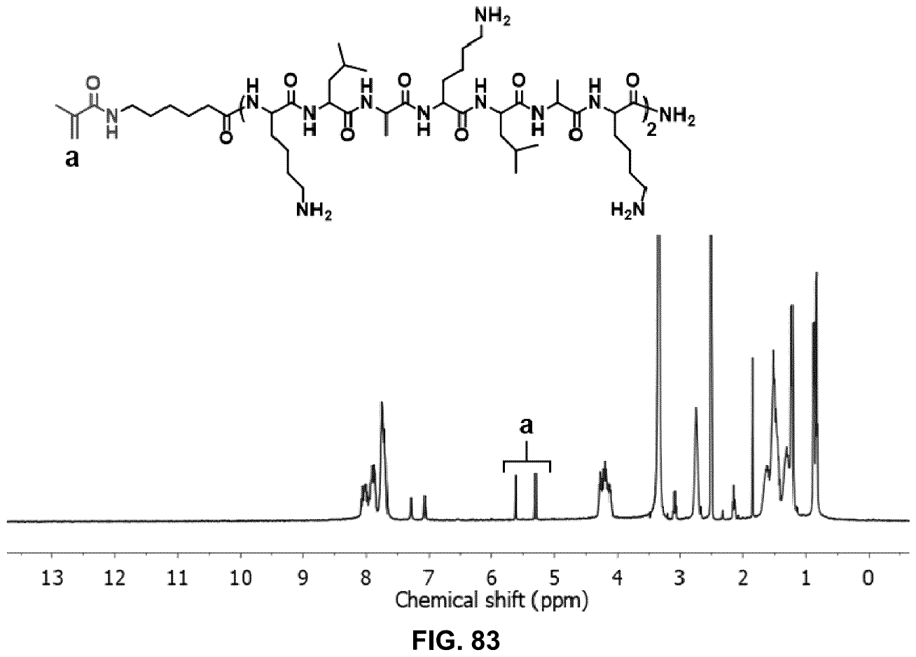

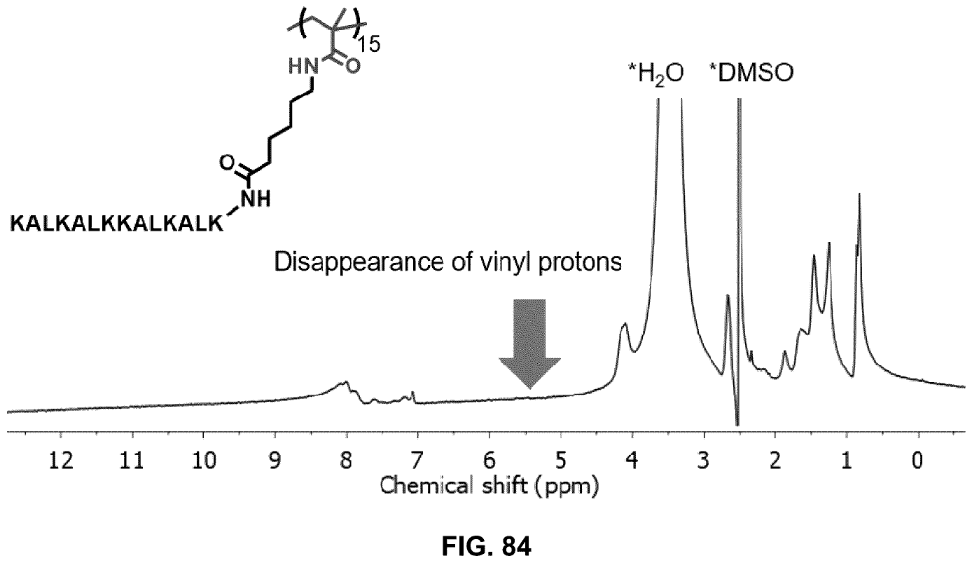

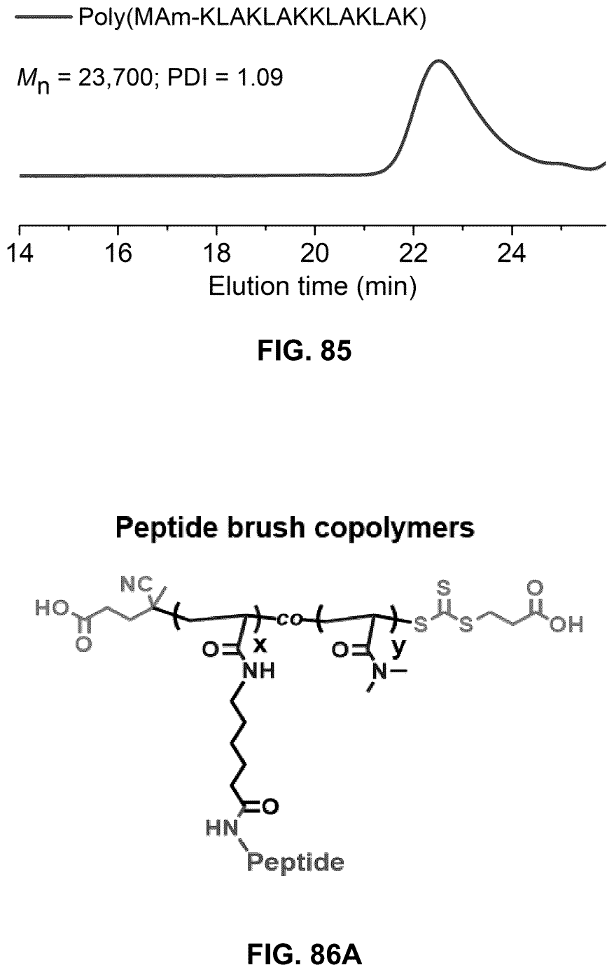

Notably, the toxicity of NP5 was significantly higher than NP3, potentially the result of the higher graft density of the KLA peptide in the hydrophilic shell on NP5 leading to enhanced multivalent interactions. In addition, the cell uptake efficiency of rhodamine B-labeled KLA peptide and NPs was investigated (FIGS. 67-69). Flow cytometry and confocal laser scanning microscopy clearly demonstrated the significantly enhanced cell uptake of the NPs over free KLA peptide.

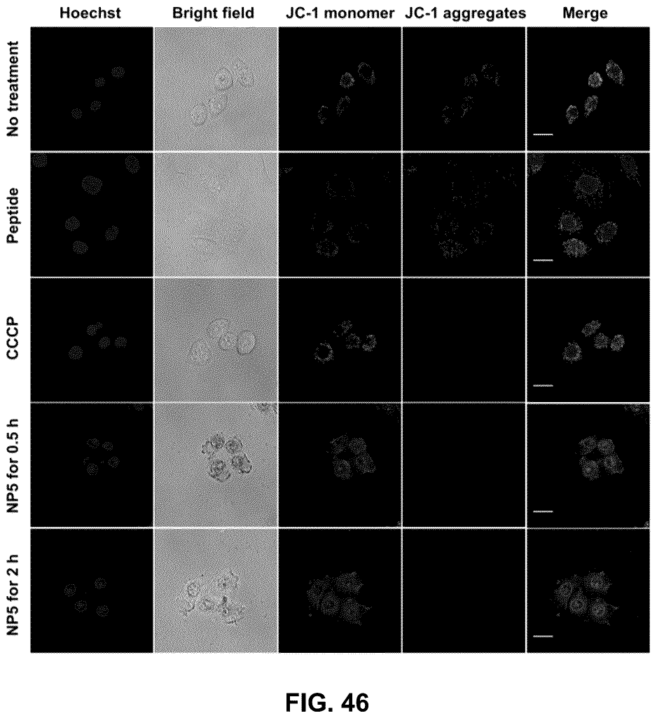

Finally, to discern the mechanism of cell death, we studied the mitochondrial membrane potential using the turn-on JC-1 probe. Mitochondria are central regulators of cellular energy and metabolism, and have the essential function of ATP synthesis by maintaining a membrane potential gradient. The JC-1 probe is green-fluorescent carbocyanine that forms red-shifted J-aggregates upon accumulation in mitochondria and has very narrow red fluorescence.^{[5b,7b]} Therefore, confocal laser scanning microscopy was utilized to compare the green and red fluorescence at the same excitation wavelength at 488 nm (FIG. 46). Hela cells incubated with free KLA peptide showed strong red fluorescence, similar to cells treated only with media, indicative of healthy mitochondria. As a comparison, almost no red emission from JC-1 J-aggregates was observed in cells treated with KLA brush polymer nanoparticles (NP5) even after 30 min incubation, confirming efficient depolarization of the mitochondria. The behavior of NP5 was similar to the commercial mitochondrial membrane potential disruptor, carbonyl cyanide 3-chlorophenylhydrazone (CCCP).

### CONCLUSION

In summary, we developed a scalable and highly modular photo-PISA approach to functional peptides displayed as hydrophilic brushes on polymeric amphiphiles packed to form micellar nanoparticles. This is a robust approach to access nanoparticles with a high-density display of peptides, tunable particle size, tunable peptide loading, and at scale (150 mg/mL). This method for packaging peptides was demonstrated with a proof-of-concept proapoptotic peptide. These results clearly demonstrate the promise of exploiting NPs with high peptide grafting densities to achieve enhanced proteolytic stability, cellular internalization, and cytotoxicity in comparison with free apoptotic peptides. We envision that many other functional peptides such as cell-penetrating and therapeutic peptides would be compatible with the photo-PISA approach to polymer brush amphiphile self-assemblies.

### References Corresponding to Example 2A

[1] B. Yu, D. Hwang, H. Jeon, H. Kim, Y. Lee, H. Keum, J. Kim, D. Y. Lee, Y. Kim, J. Chung, S. Jon, Angew. Chem. Int. Ed. 2019, 58, 2005-2010.
[2] D. J. Drucker, Nat. Rev. Drug Discov. 2020, 19, 277-289.
[3] a) T. Navaratna, L. Atangcho, M. Mahajan, V. Subramanian, M. Case, A. Min, D. Tresnak, G. M. Thurber, J. Am. Chem. Soc. 2020, 142, 1882-1894; b) M. A. Kasper, M. Glanz, A. Oder, P. Schmieder, J. P. von Kries, C. P. R. Hackenberger, Chem Sci 2019, 10, 6322-6329; c) R. Menacho-Melgar, J. S. Decker, J. N. Hennigan, M. D. Lynch, J. Control Release 2019, 295, 1-12; d) M. J. Roberts, M. D. Bentley, J. M. Harris, Adv. Drug Deliv. Rev. 2012, 64, 116-127; e) M. J. Webber, E. A. Appel, B. Vinciguerra, A. B. Cortinas, L. S. Thapa, S. Jhunjhunwala, L. Isaacs, R. Langer, D. G. Anderson, Proc. Natl. Acad. Sci. USA 2016, 113, 14189-14194; f) L. Gentilucci, R. De Marco, L. Cerisoli, Curr. Pharm. Design 2010, 16, 3185-3203; g) J. Chatterjee, C. Gilon, A. Hoffman, H. Kessler, Accounts Chem. Res. 2008, 41, 1331-1342; h) H. Itoh, K. Miura, K. Kamiya, T. Yamashita, M. Inoue, Angew. Chem. Int. Ed. 2020, 59, 4564-4571; i) Y. T. Wu, F. Villa, J. Maman, Y. H. Lau, L. Dobnikar, A. C. Simon, K. Labib, D. R. Spring, L. Pellegrini, Angew. Chem. Int. Ed. 2017, 56, 12866-12872.
[4] G. Guidotti, L. Brambilla, D. Rossi, Trends Pharmacol. Sci. 2017, 38, 406-424.
[5] a) J. Pokorski, D. C. Church, Angew. Chem. Int. Ed. 2020; b) A. P. Blum, J. K. Kammeyer, N. C. Gianneschi, Chem. Sci. 2016, 7, 989-994; c) A. P. Blum, J. K. Kammeyer, J. Yin, D. T. Crystal, A. M. Rush, M. K. Gilson, N. C. Gianneschi, J. Am. Chem. Soc. 2014, 136, 15422-15437.
[6] a) T. Suma, J. W. Cui, M. Mullner, S. W. Fu, J. Tran, K. F. Noi, Y. Ju, F. Caruso, J. Am. Chem. Soc. 2017, 139, 4009-4018; b) C. D. Spicer, C. Jumeaux, B. Gupta, M. M. Stevens, Chem. Soc. Rev. 2018, 47, 3574-3620; c) H. L. Chee, C. R. R. Gan, M. Ng, L. Low, D. G. Fernig, K. K. Bhakoo, D. Paramelle, ACS Nano 2018, 12, 6480-649.
[7] a) L. Adamiak, M. A. Touve, C. L. M. LeGuyader, N. C. Gianneschi, ACS Nano 2017, 11, 9877-9888; b) D. B. Cheng, X. H. Zhang, Y. J. Gao, L. Ji, D. Y. Hou, Z. Q. Wang, W. H. Xu, Z. Y. Qiao, H. Wang, J. Am. Chem. Soc. 2019, 141, 7235-7239; c) C. E. Callmann, M. P. Thompson, N. C. Gianneschi, Accounts Chem. Res. 2020, 53, 400-413; d) S. Hua, M. B. C. de Matos, J. M. Metselaar, G. Storm, Front Pharmacol. 2018, 9, 790.
[8] H. Sun, W. Choi, N. Zang, C. Battistella, M. P. Thompson, W. Cao, X. Zhou, C. Forman, N. C. Gianneschi, Angew. Chem. Int. Ed. 2019, 58, 17359-17364.
[9] a) C. Grazon, P. Salas-Ambrosio, E. Ibarboure, A. Buol, E. Garanger, M. W. Grinstaff, S. Lecommandoux, C. Bonduelle, Angew. Chem. Int. Ed. 2020, 59, 622-626; b) J. C. Foster, S. Varlas, L. D. Blackman, L. A. Arkinstall, R. K. O'Reilly, Angew. Chem. Int. Ed. 2018, 57, 10672-10676; c) N. J. W. Penfold, J. Yeow, C. Boyer, S. P. Armes, ACS Macro Lett 2019, 8, 1029-1054; d) S. L. Canning, G. N. Smith, S. P. Armes, Macromolecules 2016, 49, 1985-2001; e) A. M. Oliver, J. Gwyther, C. E. Boott, S. Davis, S. Pearce, I. Manners, J. Am. Chem. Soc. 2018, 140, 18104-18114; f) C. A. Figg, R. N. Carmean, K. C. Bentz, S. Mukherjee, D. A. Savin, B. S. Sumerlin, Macromolecules 2017, 50, 935-943; g) D. B. Wright, M. A. Touve, M. P. Thompson, N. C. Gianneschi, ACS Macro Lett 2018, 7, 401-405; h) M. A. Touve, C. A. Figg, D. B. Wright, C. Park, J. Cantlon, B. S. Sumerlin, N. C. Gianneschi, ACS Central Sci. 2018, 4, 543-547; i) C. A. Figg, A. Simula, K. A. Gebre, B. S. Tucker, D. M. Haddleton, B. S. Sumerlin, Chem. Sci. 2015, 6, 1230-1236; j) D. Le, M. Dilger, V. Pertici, S. Diabate, D. Gigmes, C. Weiss, G. Delaittre, Angew. Chem. In. Ed. 2019, 58, 4725-4731.
[10] a) J. Yeow, C. Boyer, Adv. Sci. 2017, 4, 1700137; b) L. D. Blackman, S. Varlas, M. C. Arno, Z. H. Houston, N. L. Fletcher, K. J. Thurecht, M. Hasan, M. I. Gibson, R. K. O'Reilly, ACS Central Sci. 2018, 4, 718-723; c) J. B. Tan, H. Sun, M. G. Yu, B. S. Sumerlin, L. Zhang, ACS Macro Lett 2015, 4, 1249-1253; d) N. Corrigan, J. Yeow, P. Judzewitsch, J. T. Xu, C. Boyer, Angew. Chem. Int. Ed. 2019, 58, 5170-5189.
[11] M. Cieslewicz, J. J. Tang, J. L. Yu, H. Cao, M. Zavaljevski, K. Motoyama, A. Lieber, E. W. Raines, S. H. Pun, Proc. Natl. Acad. Sci. USA 2013, 110, 15919-15924.
[12] a) J. T. Xu, K. Jung, A. Atme, S. Shanmugam, C. Boyer, J. Am. Chem. Soc. 2014, 136, 5508-5519.; b) M. Chen, M. J. Zhong, J. A. Johnson, Chem. Rev. 2016, 116, 10167-10211.
[13] a) D. B. Thomas, B. S. Sumerlin, A. B. Lowe, C. L. McCormick, Macromolecules 2003, 36, 1436-1439; b) D. B. Thomas, A. J. Convertine, R. D. Hester, A. B. Lowe, C. L. McCormick, Macromolecules 2004, 37, 1735-1741.
[14] a) W. Zhou, Q. W. Qu, Y. Y. Xu, Z. S. An, ACS Macro Lett 2015, 4, 495-499; b) T. Luckerath, K. Koynov, S. Loescher, C. J. Whitfield, L. Nuhn, A. Walther, C. Barner-Kowollik, D. Y. W. Ng, T. Weil, Angew. Chem. Int. Ed. 2020, doi: 10.1002/anie.201916177.
[15] M. P. Chien, A. M. Rush, M. P. Thompson, N. C. Gianneschi, Angew. Chem. Int. Ed. 2010, 49, 5076-5080.
[16] a) J. Lee, E. T. Oh, H. Lee, J. Kim, H. G. Kim, H. J. Park, C. Kim, Bioconjug. Chem. 2020, 31, 43-50; b) D. J. Toft, T. J. Moyer, S. M. Standley, Y. Ruff, A. Ugolkov, S. I. Stupp, V. L. Cryns, ACS Nano 2012, 6, 7956-7965.
[17] a) F. Jia, X. G. Lu, X. Y. Tan, D. L. Wang, X. Y. Cao, K. Zhang, Angew. Chem. Int. Ed. 2017, 56, 1239-1243; b) F. Jia, X. G. Lu, D. L. Wang, X. Y. Cao, X. Y. Tan, H. Lu, K. Zhang, J. Am. Chem. Soc. 2017, 139, 10605-10608.
[18] B. A. Ji, C. D. Logsdon, Gastroenterology 2011, 141, 1972-1975.
[19] J. M. G. Artigas, M. R. A. Faure, M. E. Garcia, A. M. B. Gimeno, Postgrad. Med. J. 1981, 57, 219-222.
[20] M. Giacomello, A. Pyakurel, C. Glytsou, L. Scorrano, Nat. Rev. Mol. Cell Bio. 2020, 21, 202-224.

### Example 2B: Proapoptotic Peptide Brush Polymer Nanoparticles via Photoinitiated Polymerization-Induced Self-Assembly-Supporting Information

### 1. Materials

All amino acids used to prepare peptides by solid phase peptide synthesis (SPPS) were obtained from AAPPTec, Chem-Impex, and NovaBiochem. N,N,N′,N″,N″-Pentamethyldiethylenetriamine (PMDETA, 99%), eosin Y disodium salt (dye content >85%), 6-Fmoc-amino hexanoic acid (97%), O,O′-1,3-Propanediylbishydroxyamine·2HCl (crosslinker, >99%), and acetate buffer (0.1 M, pH 5) were purchased from Sigma Aldrich and used without purification. Diacetone acrylamide (DAAm, 99%) was purchased from Sigma Aldrich and purified by crystallization twice from ethyl acetate and once from hexane before use.¹ 4-((((2-Carboxyethyl)thio) carbonothioyl)thio)-4-cyanopentanoic acid (water-soluble RAFT agent, 95%) was purchased from Combi-Blocks and used without further purification. Methacryloxyethyl thiocarbamoyl rhodamine B was purchased from Polysciences, Inc. Rhodamine B labeled KLA peptide (Rho-KLA) was synthesized according to our previous report.² Trypsin was purchased from Sigma. LED strip light (450 nm) was purchased from Amazon. CellTiter-Blue® was purchased from Promega Corporation. Dulbecco's Phosphate Buffered Saline (without Ca²⁺, Mg²⁺) was purchased from Corning Cellgro. Dulbecco's modified Eagle medium (DMEM), fetal bovine serum (FBS), Hoechst 33342, and JC-1 probe were purchased from ThermoFisher Scientific.

### 2. Methods

¹H Nuclear Magnetic Resonance (¹H NMR): ¹H NMR spectra were recorded on a Varian Inova spectrometer (500 MHz) in DMSO-d₆. Chemical shifts are given in ppm downfield from tetramethylsilane TMS.

Analytical High-Performance Liquid Chromatography (HPLC): Analytical HPLC analysis of peptides was performed on a Jupiter 4μ Proteo 90 Å Phenomenex column (150×4.60 mm) using a Hitachi-Elite LaChrom L-2130 pump equipped with UV-Vis detector (Hitachi-Elite LaChrom L2420).

Preparative HPLC: Armen Glider CPC preparatory HPLC was used to purify the peptides. The solvent system consists of (A) 0.1% TFA in water and (B) 0.1% TFA in acetonitrile.

Electrospray Ionization Mass Spectrometry (ESI-MS): ESI-MS spectra of peptides were collected using a Bruker Amazon-SL spectrometer configured with an ESI source in both negative and positive ionization mode.

Dry-State Transmission Electron Microscopy (TEM): Twenty microliters of samples were applied onto a 400 mesh carbon grids (Ted Pella, INC.). The grids were observed on a Hitachi HT 7700 microscope operating at 120 kV. The images were recorded with a slow-scan charge-coupled device (CCD) camera (Veleta 2k×2k).

Cryogenic Transmission Electron Microscopy (Cryo-TEM): Five microliters of sample were applied onto a 200 mesh lacey carbon grid (Ted Pella, INC.) that had been glow discharged for 30 seconds. The samples were manually blotted and vitrified in ethane before imaging on a JEOL 1230 microscope operating at 100 kV. Images were acquired on a One View CCD camera.

Gel Permeation Chromatography (GPC): GPC measurements were performed on a set of Phenomenex Phenogel 5μ, 1K-75K, 300× 7.80 mm in series with a Phenomex Phenogel 5u, 10K-1000K, 300× 7.80 mm columns with HPLC grade solvents as eluents: dimethylformamide (DMF) with 0.05M of Li Br at 60 °C. Detection consisted of a Hitachi UV-Vis Detector L-2420, a Wyatt Optilab T-rEX refractive index detector operating at 658 nm and a Wyatt DAWN® HELEOS® II light scattering detector operating at 659 nm. Absolute molecular weights and polydispersities were calculated using the Wyatt ASTRA software with dn/dc values determined by assuming 100% mass recovery during GPC analysis.

Dynamic Light Scattering (DLS) and Zeta Potential Analysis: DLS and zeta potential analyses were conducted at room temperature on a Zetasizer Nano-ZS (Malvern). The laser for DLS was at a wavelength of 633 nm.

Circular Dichroism Spectrophotometry (CD): CD spectra were measured using a Jasco J815 spectrometer and each sample was measured from 190 to 260 nm with a slit width of 1 nm, scanning at 1 nm intervals with a 1s integration time. Measurements were taken 3x at 25 °C and then averaged to give the spectra. Notably, the peptide and polymer were dissolved in deionized water to a concentration of 100 UM (with respect to peptide concentration).

Flow Cytometry: The cell uptake study was analyzed via flow cytometry using a BD FacsAria Ilu 4-Laser flow cytometer (Becton Dickinson Inc., USA). Mean fluorescence intensity and PE-A-histogram data was prepared for presentation using FlowJo v10.

Confocal Laser Scanning Microscopy (CLSM): Imaging was accomplished using LEICA SP5 II laser scanning confocal microscope with a 63x oil immersion objective at 1.5× optical zoom. All the images were Z-stack images. Slice thickness was 0.26 μm with a scan size of 1024×1024 pixels and a scan speed of 400 Hz. The cell nuclei (stained with DAPI) was accomplished using a 405 nm laser with a 15% laser power. The cell membrane (stained with Wheat Germ Agglutinin, Alexa Fluor 488 Conjugate) was accomplished using a 488 nm laser with a 12% laser power. Cell imaging for Rhodamine fluorescence was accomplished using a 543 nm laser with an 8% laser power.

Fluorescence Measurement: CellTiter-Blue® fluorescence measurements were recorded using a Perkin Elmer EnSpire multimode Plate Reader.

### 3. Experimental

### 3.1 Preparation of KLA Peptide Monomer via Solid-Phase Peptide Synthesis (SPPS)

KLA peptide acrylamide was synthesized on Rink resin (0.67 mmol/g) using standard FMOC SPPS procedures on an AAPPTec Focus XC automated synthesizer. A typical SPPS procedure included deprotection of the N-terminal Fmoc group with 20% 4-methyl-piperidine in DMF (1×20 min, followed by 1×5 min), and 30 min amide couplings (twice) using 3.0 equiv. of the Fmoc-protected amino acid, 2.9 equiv. of HBTU and 6 equiv. of DIPEA. After that, peptide monomers were prepared by amide coupling to Fmoc-6-aminohexanoic acid, followed by Fmoc deprotection and final amidation with acrylic acid (3 equiv.) in the presence of HBTU (2.9 equiv.), and DIPEA (6 equiv.).

### 3.2 Synthesis of MacroCTA Via PET-RAFT Polymerization

In a typical aqueous photo-induced RAFT polymerization for making poly(KLAAm₁₀-co-DMA₃₀) macroCTA, KLA peptide acrylamide monomer (30 mg, 10 equiv.) and DMA (4.7 mg, 30 equiv.) were dissolved in 150 μL of acetate buffer (0.1 M, pH 5). Then, water-soluble RAFT agent (0.55 mg, 1.0 equiv.) was added into the reaction mixture. Following that, 10 μL (0.063 mg, 0.05 equiv.) of eosin Y disodium salt stock solution (6.3 mg in 1 mL of acetate buffer) and PMDETA (0.41 mg, 1.0 equiv.) were added. The solution was degassed by N₂ flow for 30 min and then placed into the photo-reactor (450 nm, 2.8 mW/cm²) for 12 h. After polymerization, the macroCTA solution was directly used for photo-PISA (vide infra) without purification.

### 3.3 Photoinitiated Polymerization-Induced Self-Assembly (Photo-PISA)

In a typical photo-PISA protocol for preparing NP1, DAAm (21 mg, 70 equiv.) and DMA (4.7 mg, 30 equiv.) were added into the macroCTA solution which was made in section 3.2 (vide supra). Next, 180 μL of acetate buffer (0.1 M, pH 5) was added to achieve a solids content of 15 wt. %. The solution was degassed by N₂ flow for 30 min and then placed into the photo-reactor (450 nm, 2.8 mW/cm²) for 12 h. After PISA, the NPs were further purified via dialysis into deionized water.

### 3.4 Preparation of Rhodamine B-Labeled NPs

In all the cases of preparing rhodamine-labeled NPs, one equiv. of rhodamine B to RAFT agent was used, ensuring that on average one dye was attached to each polymer chain. In a typical synthesis of rhodamine B-labeled NP3, KLA peptide acrylamide monomer (30 mg, 10 equiv.), DMA (4.7 mg, 30 equiv.), and methacryloxyethyl thiocarbamoyl rhodamine B (1.4 mg, 1.0 equiv.) were dissolved in 150 μL of acetate buffer (0.1 M, pH 5). Then water-soluble RAFT agent (0.55 mg, 1.0 equiv.) was added into the reaction mixture. Following that, 10 μL (0.05 equiv.) of eosin Y disodium salt stock solution (6.3 mg in 1 mL of acetate buffer) and PMDETA (0.41 mg, 1.0 equiv.) were added. The solution was degassed by N₂ flow for 30 min and then placed into the photo-reactor (450 nm, 2.8 mW/cm²) for 12 h. After polymerization, the macroCTA solution was directly used in next step (photo-PISA) to prepare rhodamine B-labeled NP3. DAAm (84 mg, 280 equiv.) and DMA (19 mg, 120 equiv.) were added into the macroCTA solution. Next, 630 L of acetate buffer (0.1 M, pH 5) was added to achieve a solids content of 15 wt. %. The solution was degassed by N₂ flow for 30 min and then placed into the photo-reactor (450 nm, 2.8 mW/cm²) for 12 h. After PISA, the rhodamine B labeled NPs were further purified via dialysis in deionized water.

3.5 Synthesis of PolyDMA₄₀-b-Poly(DAAm₇₀-co-DMA₃₀)

DMA (12.6 mg, 40 equiv.) were dissolved in 300 μL of acetate buffer (0.1 M, pH 5). Then, water-soluble RAFT agent (1.1 mg, 1.0 equiv.) was added into the reaction mixture. Following that, 20 μL (0.126 mg, 0.05 equiv.) of eosin Y disodium salt stock solution (6.3 mg in 1 mL of acetate buffer) and PMDETA (0.82 mg, 1.0 equiv.) were added. The solution was degassed by N₂ flow for 30 min and then placed into the photo-reactor (450 nm, 2.8 mW/cm²) for 12 h. After polymerization, DAAm (42 mg, 70 equiv.) and DMA (9.4 mg, 30 equiv.) were added into the macroCTA solution. Next, 75 UL of acetate buffer (0.1 M, pH 5) was added to achieve a solids content of 15 wt. %. The solution was degassed by N₂ flow for 30 min and then placed into the photo-reactor (450 nm, 2.8 mW/cm²) for 12 h. After PISA, the NPs were further purified via dialysis into deionized water.

### 3.6 Trypsin-Induced Cleavage Experiments

For protease-triggered cleavage experiments, the molar ratio of trypsin to peptide was set to 1:2000. Moreover, the temperature was set to 37 °C to match the body temperature. For example, poly [(KLAAm₁₀-C₀-DMA₃₀)-b-(DAAm₇₀-co-DMA₃₀)] (NP1, 0.9 mg, 0.2 μmol with respect to peptides, 2000 equiv.) was dissolved in 1 ml of DPBS solution. Then trypsin (0.023 mg, 0.1 nmol, 1 equiv.) was added into the polymer solution which was stirred in a preheated oil b ath at 37 °C. During the cleavage, aliquots were taken for HPLC analysis at predetermined time points.

### 3.7 Cell Viability Assay

Hela cells were grown in Dulbecco's modified Eagle medium (DMEM) supplemented with 10% fetal bovine serum (FBS), 1% penicillin-streptomycin. Cells were maintained at 37 °C and 5% CO₂ with a relative humidity of 95%. Hela cells were plated in 96-well plates at a density of 5000 per well and then left to attach for 24 h. Subsequently, the cells were treated with the polymers of various concentrations for the desired time followed by washing 3 times with PBS. Then CellTiter-Blue® at 10% (v/v) in complete media was added to each well and incubated for 2 h to allow the live cells to convert resazurin to fluorescent resorufin. The fluorescent signal (Excitation wavelength: 560 nm; Emission wavelength: 600 nm) was then analyzed by a plate reader. Three replicates were performed for each independent sample. 10% DMSO was used as a positive control and untreated cells in complete medium as a negative control. Viability is reported as a percentage of untreated cells.

### 3.8 Confocal Laser Scanning Microscopy for Uptake in Hela Cells

HeLa cells were plated in a 4-chamber 35 mm round glass-bottom dishes at a density of 50,000 per well. Cells were incubated for 24 hours in a 5% CO₂ atmosp here at 37 °C. 500 μL of Rho-KLA peptide, Rho-NP3, and Rho-NP5 (0.25 UM with respect to rhodamine for each material) in 10% FBS DMEM media without phenol red were incubated with the cells for 24 hours, respectively. After washing with DPBS to remove the residual peptides and nanoparticles, 500 μL of Wheat Germ Agglutinin (5 μg/mL) conjugated with Alexa Fluor 488 was added to each well, then fixed with a 4% paraformaldehyde solution for 15 min at room temperature. The cells were washed with DPBS and stained by DAPI for 20 min at room temperature.

### 3.9 Flow Cytometry for Uptake Ability in Hela Cells

For cellular uptake measurements, Hela cells were plated in 12-well plates at a density of 1,000,000 per well and incubated for 24 hours in a 5% CO₂ atmos phere at 37 °C. 500 μL of 0.25 UM (with respect to rhodamine) Rho-KLA peptide, Rho-NP3, and Rho-NP5 in 10% FBS DMEM media without phenol red were incubated with the cells for 24 hours respectively. After triple washing with DPBS, 500 μL of 0.25% Trypsin-EDTA was added to each well for 10 min at 37 °C. Cells were fixed with a 4% paraformaldehyde solution for 15 min at room temperature.

### 3.10 Mitochondria Membrane Potential of Hela Cells

Hela cells were plated in 4-well, glass bottom dishes at 30,000 cells per well in 500 μL medium. Hela cells were seeded for 24 h before treatment with nanoparticle suspensions for 24 or 72 h. The cells were stained with 2 UM of JC-1 at 37 °C for 15 minutes, washed with PBS to remove any membrane-bound, non-internalized fluorophores, and returned to complete medium. For the positive control group, the cells were incubated with 50 M CCCP and 2 μM of JC-1 probe in DPBS solution simultaneously for 15 min. Before the confocal observation, 1 drop of Hoechst 33342 dye was added to stain the nuclei. Confocal microscope was employed to observe the fluorescence of JC-1 monomer, J-aggregates and Hoechst dye.

### References Corresponding to Example 2B

1. Figg, C. A.; Carmean, R. N.; Bentz, K. C.; Mukherjee, S.; Savin, D. A.; Sumerlin, B. S., Tuning Hydrophobicity To Program Block Copolymer Assemblies from the Inside Out. Macromolecules 2017, 50 (3), 935-943.
2. Sun, H.; Choi, W.; Zang, N.; Battistella, C.; Thompson, M. P.; Cao, W.; Zhou, X.; Forman, C.; Gianneschi, N. C., Bioactive Peptide Brush Polymers via Photoinduced Reversible-Deactivation Radical Polymerization. Angew. Chem. Int. Ed. 2019, 58 (48), 17359-17364.

### Example 3: A Scalable Method For Preparing Peptide-Shell Polymer Nanoparticles Via Photoinitiated Polymerization-Induced Self-Assembly

Synthetic oligopeptides represent a class of powerful therapeutics because of their biocompatibility, straightforward synthesis, predictable metabolism, and high degree of modularity in molecular design. However, these advantages are typically compromised by natural processes prevalent in cells and tissues that have evolved to degrade them. Moreover, cell internization of peptides is typically inefficient, often requiring selective cell surface interactions through the use of cell penetrating sequences. These inherent downsides of oligopeptides as drugs have tremendously hampered their translations into clinic use. To tackle these challenges, we herein demonstrate a scalable, one-pot approach to peptide-based brush polymer amphiphile assemblies. For this, we employed one-pot photoinitiated polymerization-induced self-assembly (photo-PISA) to access spheric nanoparticles. The resulting materials are characterized by a high-density display of apoptotic peptides (amino acid sequence: KLAKLAKKLAKLAK) (SEQ ID NO:3) in the hydrophilic shell. Emergent properties include both proteolytical resistance and bioactivity, including cell penetration and apoptotic efficiency. All of these features were significantly higher for the peptide brush polymer nanoparticles compared to their linear peptide analogues. These results demonstrate the promise of exploiting polymer nanoparticles with high peptide grafting densities to achieve enhanced proteolytic stability and cytotoxicity in comparison with free apoptotic peptides.

Applications include peptide delivery systems for treating human diseases, high performance adhesive materials, and anti-fouling coatings.

Advantages: In tradition, solvent-switch strategies are used to access self-assembled nanostructures of peptide-polymer amphiphiles. However, the scale or the concentration of the products is limited to less than 20 mg/mL by these classic methods. In our invention, we solved this problem by using polymerization-induced self-assembly approach, which led to the at scale production of high concentrations of peptide brush polymer nanoparticles up to 150 mg/mL. This scale is very important for the translation of these promising nanomedicine into large animal models and eventually clinic trial. Oligopeptides are very unstable in the presense of protease which are everywhere in vivo. In our invention, peptide brush polymer nanoparticles adopt the three-dimensional spatial arrangement of peptides. This not only can improve the proteolytic stability of peptides, but also can enhance their biological activities such as cell binding and penetration via multivalent effects.

Herein we report photoinitiated polymerization-induced self-assembly (photo-PISA) of spherical micelles consisting of proapoptotic peptide-polymer amphiphiles. The one-pot synthetic approach yielded micellar nanoparticles at high concentrations and at scale (150 mg/mL) with tunable peptide loadings up to 48 wt. %. The size of the micellar nanoparticles was tuned by varying the lengths of hydrophobic and hydrophilic building blocks. Importantly, the peptide-functionalized nanoparticles imbued peptides, such as the proapoptotic “KLA” peptides (amino acid sequence: KLAKLAKKLAKLAK) (SEQ ID NO: 3), with two key properties otherwise not inherent to the sequence: 1) proteolytic resistance compared to the oligopeptide alone; 2) significantly enhanced cell uptake permeability by multivalent display of KLA peptide brushes. The result was demonstrated improved apoptosis efficiency in Hela cells. These results highlight the potential of photo-PISA in the large-scale synthesis of functional, proteolytically resistant peptide-polymer conjugates for intracellular delivery. We envision that many other functional peptides such as cell-penetrating, anti-fouling, and therapeutic peptides would be compatible with the photo-PISA approach to polymer brush amphiphile self-assemblies.

Included herein is a new peptide delivery system which can significantly enhance the life-time (stability), cell penetration, and efficacy of peptide therapeutics. Currently, pharmaceutical industry can achieve enhanced stability of peptides by using strategies such as PEGlyzation, lipidation, and cyclization, among others. However, these methods compromise the bioactivity or function of modified peptides as a result of the alteration of chemical identity and connectivity in amino acids.

### References Corresponding to Example 3

1. Wright, D. B.; Proetto, M. T.; Touve, M. A.; Gianneschi, N. C., Ring-opening metathesis polymerization-induced self-assembly (ROMPISA) of a cisplatin analogue for high drug-loaded nanoparticles. Polym. Chem. 2019, 10 (23), 2996-3000.
2. Blackman, L. D.; Varlas, S.; Arno, M. C.; Houston, Z. H.; Fletcher, N. L.; Thurecht, K. J.; Hasan, M.; Gibson, M. I.; O'Reilly, R. K., Confinement of Therapeutic Enzymes in Selectively Permeable Polymer Vesicles by Polymerization-Induced Self-Assembly (PISA) Reduces Antibody Binding and Proteolytic Susceptibility. ACS Cent. Sci. 2018, 4 (6), 718-723.
3. Liu, X.; Sun, M.; Sun, J.; Hu, J.; Wang, Z.; Guo, J.; Gao, W., Polymerization Induced Self-Assembly of a Site-Specific Interferon α-Block Copolymer Conjugate into Micelles with Remarkably Enhanced Pharmacology. J. Am. Chem. Soc. 2018, 140 (33), 10435-10438.
4. Le, D.; Wagner, F.; Takamiya, M.; Hsiao, I.; Gil Alvaradejo, G.; Strahle, U.; Weiss, C.; Delaittre, G., Straightforward access to biocompatible poly(2-oxazoline)-coated nanomaterials by polymerization-induced self-assembly. Chem. Commun, 2019, 55, 3741-3744.

### Example 4: Exemplary Methods and Descriptions of Homopolymers Via Photo-RDRP

Typical protocol of making peptide brush homopolymers via photo-RDRP: Synthesis of poly(MAm-KLAKLAKKLAKLAK) (SEQ ID NO:3): In a typical protocol, the peptide methacrylamide monomer MAm-KLAKLAKKLAKLAK (SEQ ID NO:3) (31 mg, 15 equiv.) was dissolved in 180 μL of sodium acetate buffer (0.1 M, pH=5). Thereafter, 10 μL (1.0 equiv.) of water-soluble CTA stock solution (3.8 mg in 100 μL of DMSO) was added into the reaction mixture. Following that, the photoinitiator sodium phenyl-2,4,6-trimethylbenzoylphosphinate (SPTP, 0.12 mg, 0.3 equiv.) was added into the solution by injecting 10 μL of SPTP stock solution (1.2 mg in 0.1 mL of acetate buffer) were added. The solution was degassed by N₂ flow for 20 min and then placed into the photo-reactor (365 nm) for 18 h. After the polymerization, the polymer product was purified by dialysis into DIW, followed by lyophilization.

In some literature, peptide brush homopolymers made by traditional or thermally-initiated RDRP methods are limited to short and simple peptides such as MARGD and VPGVG which consist of only five amino acids. 1-2 The RDRP based synthesis of peptide brush homopolymers with long and complex peptide sequence still remain unexplored. Leveraging photo-RDRP, we have now invented a synthetic approach to peptide brush homopolymers consisting of long peptide sequences (e.g., up to 15 amino acids). This approach is versatile to a library of long and complicated peptide sequences, including but not limited to GPLGLAGGWGER (SEQ ID NO: 12), GALTPRGADSGSG (SEQ ID NO:2), GSGKEFGADSGSG (SEQ ID NO:4), and KLAKLAKKLAKLAK (SEQ ID NO:3). In addition, the monomer conversions were quantitative (>99%) in all cases, indicative of robustness of this approach.

### REFERENCES CORRESPONDING TO EXAMPLE 4

1. Thang et al. Polym. Chem., 2018, 9, 1780-1786.
2. Cameron et al. Macromolecules 2007, 40, 17, 6094-6099.

### Example 5: Photo-RDRP Versus Thermally Initiated RAFT for Peptide Brush Polymers

“Graft through” reversible deactivation radical polymerization (RDRP) of methacrylic macromonomers such as long and bulky peptide methacrylamide is synthetically challenging because the repulsion between bulky side chains would lead to a reduced enthalpy of polymerization (ΔH) due to C—C bond stretching and bond-angle deformation in the vinyl polymer backbone.¹ The decreased ΔH would result in a smaller gain in free energy of polymerization (ΔG) and thus generate competition between polymerization and depolymerization. In view of this, graft through RDRP of peptide macromonomers would suffer from a rather high equilibrium monomer concentration ([M]_{eq}) at which the polymerization rate is equal to depolymerization rate.² To mitigate the issue of high equilibrium concentration and achieve a high monomer conversion, we contemplate strategies including (i) increase the initial monomer concentration; (ii) increase the pressure; (iii) use a poor solvent for the side chain; and (iv) lower the reaction temperature.^{1,3}

Traditional or thermally initiated reversible addition-fragmentation transfer (RAFT) polymerizations are typically performed at temperatures above 50 °C and have been proven successful for controlled polymerization of commercially available monomers such as methyl methacrylate and dimethylacrylamides which have small molecular weights.⁴ While the high temperatures would lead to high equilibrium monomer concentrations, one can still achieve high monomer conversions of those small molecular monomers by increasing the initial monomer concentrations. However, this strategy cannot be applied for the RAFT polymerization of vinyl macromonomer such as long and complex peptide monomers (e.g., having ≥5 amino acid groups, having ≥6 amino acid groups, having ≥7 amino acid groups, having ≥8 amino acid groups, having ≥9 amino acid groups, having ≥10 amino acid groups, having ≥11 amino acid groups, or having ≥12 amino acid groups) because of the upper solubility limit of the bulky peptide macromonomer. For example, the maximum concentration of KLA peptide acrylamide monomer (amino acid sequence: KLAKLAKKLAKLAK) (SEQ ID NO: 3) is about 100 mM in water.⁵ This is tremendously lower than the upper solubility limits of small molecular monomers (e.g., dimethylacrylamide: 9700 mM in bulk).

In the photo-RAFT methods we develop and disclose throughout this application for the synthesis of peptide brush homopolymers, room temperature can be used and thus significantly decrease the equilibrium monomer concentration (Van't hoff equation, see below), favoring the polymerization. Therefore, in methods disclosed herein, a large fraction of monomers can be polymerized before reaching the equilibrium, leading to a high monomer conversion. This is inaccessible by thermally-initiated RAFT polymerizations.

Van't hoff equation:

$${ln [ M ]}_{e q} = \frac{Δ H}{R T} - \frac{{S}^{0}}{R}$$

### References Corresponding to Example 5

1. Matyjaszewski et al. ACS Macro Lett. 2020, 9, 1303-1309.
2. Gramlich et al. Polym. Chem. 2018, 9, 2328-2335.
3. (a) Janata et al. Macromolecules 2014, 47 (21), 7311-7320; (b) Ivin et al. J. Polym. Sci., Part A: Polym. Chem. 2000, 38 (12), 2137-2146.
4. Perrier et al. Macromolecules 2017, 50, 19, 7433-7447.
5. Gianneschi et al. Angew. Chem. Int. Ed. 2019, 58, 17359-17364.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every polymer, composition, formulation, and method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1.-86. (canceled)

87. A peptide brush polymer comprising:
at least 5 peptide-containing repeating units; wherein each peptide-containing repeating unit comprises a poly(meth)acrylamide or poly(meth)acrylate polymer backbone group directly or indirectly covalently linked to a polymer side chain group comprising a peptide moiety;
wherein:
the peptide brush polymer is characterized by a degree of polymerization of at least 10 and a peptide graft density of 50% to 100%; and
each peptide moiety has at least 10 amino acid groups.

88. The peptide brush polymer of claim 87, wherein each peptide moiety independently comprises a sequence having at least 80% sequence homology with SEQ ID NO: 1 (GPLGLAGGWGERDGS), SEQ ID NO:2 (GALTPRGADSGSG), SEQ ID NO:3 (KLAKLAKKLAKLAK), SEQ ID NO:4 (GSGKEFGADSGSG), SEQ ID NO:5 (GPLGLAGG), SEQ ID NO:6 (HVLVMSATKKKK), SEQ ID NO:7 (GGGCYFQNCPKG) (Terlipressin), SEQ ID NO: 8 (DRVYIHPF) (Angiotensin 2), SEQ ID NO:9 (AQYQDKLAR) (DA1), SEQ ID NO: 10 (GVi(allo)SQIRP) (ABT898), SEQ ID NO: 11 (KVPRNQDWL) (gp100), or SEQ ID NO: 12 (GPLGLAGGWGER).

89. The peptide brush polymer of claim 87, wherein the peptide brush polymer is characterized by formula FX6A or FX6B:
Q¹-[B¹]ₘ-Q² (FX6A); or
Q¹-[B¹]^{m}−/−[B²]ₙ-Q² (FX6B); wherein:
each B¹ is independently a peptide-containing polymer block;
each B² is independently a peptide-free polymer block;
m is an integer greater than or equal to 1;
n is 0 or an integer greater than or equal to 1;
the symbol “/” indicates that the units separated thereby are covalently linked randomly or in any order; and
each of Q¹ and Q² is independently a polymer terminating group.

90. The peptide brush polymer of claim 89, wherein each B¹ is characterized by the formula (FX7): 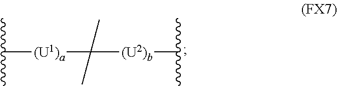
wherein:
each U¹ is independently a peptide-containing repeating unit;
each U² is independently a peptide-free repeating unit;
a is an integer selected from the range of 2 to 100;
b is 0 or an integer selected from the range of 2 to 100; and
the symbol “/” indicates that the units separated thereby are covalently linked randomly or in any order.

91. The peptide brush polymer of claim 90, wherein each U¹ is independently characterized by the formula FX8A or FX8B and each U², if present, is independently characterized by the formula FX9A or FX9B: 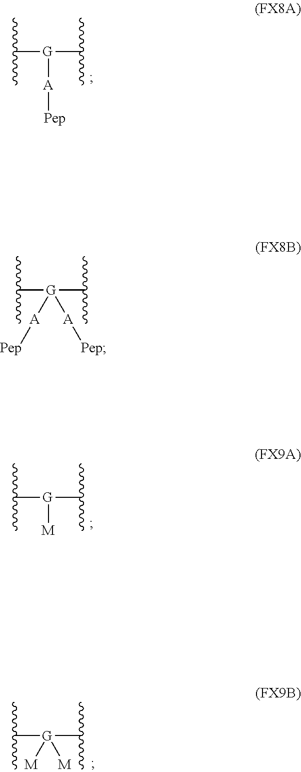
wherein:
each G is independently a polymer backbone group;
each A is independently a covalent anchor group;
each Pep is independently a peptide moiety; and
each M is independently an alkyl group, an alkenylene group, an arylene group, an alkoxy group, an acyl group, a carboxyl group, an aliphatic group, an amide group, an aryl group, an amine group, an ether group, a ketone group, an ester group, or combinations thereof.

92. The peptide brush polymer of claim 91, wherein each G is independently characterized by formula FX10A, FX10B, FX10C, FX10D, FX10E, or FX10F: 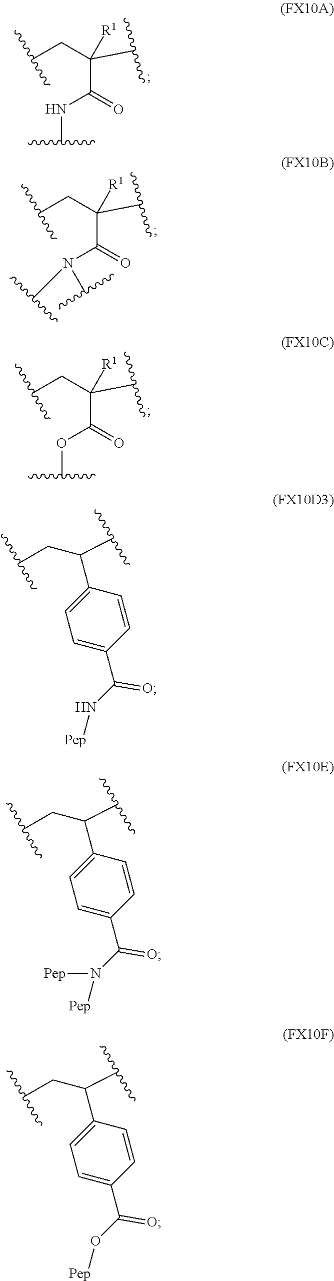
wherein:
R¹ is a hydrogen or a methyl group.

93. The peptide brush polymer of claim 91, wherein each A is independently selected from the group consisting of a single bond, an oxygen, and one or more substituted or substituted groups having an 5 alkyl group, an alkenylene group, an arylene group, an alkoxy group, an acyl group, a carboxyl group, an aliphatic group, an amide group, an aryl group, an amine group, an ether group, a ketone group, an ester group, a triazole group, a diazole group, or a pyrazole group.

94. The peptide brush polymer of claim 93, wherein each A is independently characterized by formula FX3A, FX3B, or FX3C; 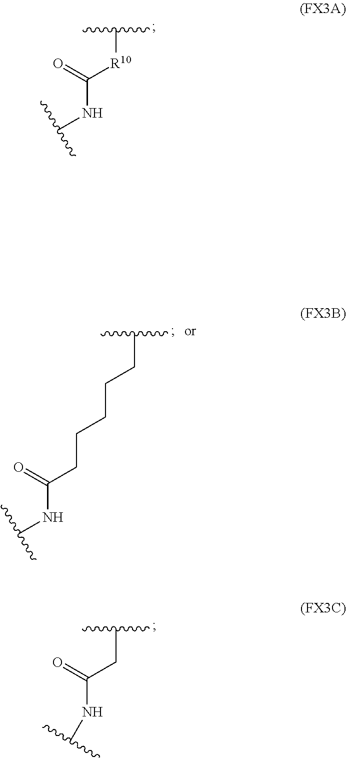
wherein: R¹⁰ is a substituted or unsubstituted C₁-C₁₀ alkyl.

95. The peptide brush polymer of claim 91, wherein the peptide brush polymer is characterized by formula FX11A or FX11B: 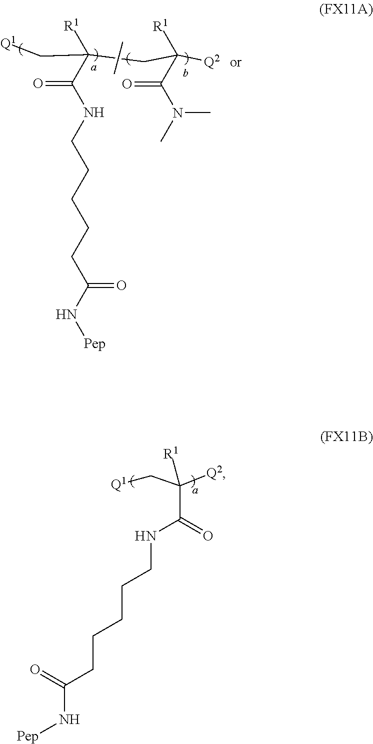
wherein R¹ is a hydrogen or a methyl group.

96. The peptide brush polymer of claim 90, wherein each of Q¹ and Q² is independently a hydrogen or characterized by formula FX14A or FX14B:

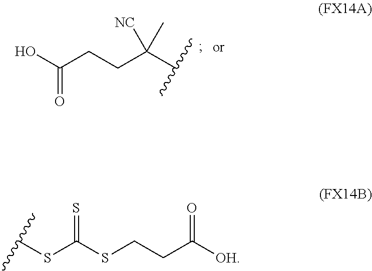

97. The peptide brush polymer of claim 91, wherein the peptide brush polymer is characterized by formula FX12:

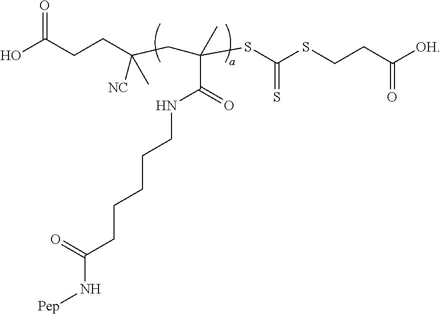

98. The peptide brush polymer of claim 89, wherein each peptide-containing polymer block is hydrophilic.

99. The peptide brush polymer of claim 89, wherein each peptide-free polymer block is hydrophobic.

100. The peptide brush polymer of claim 89, wherein each peptide-free polymer block comprises poly(methyl methacrylate) or poly(n-butyl acrylate).

101. The peptide brush polymer of claim 87, wherein the peptide brush polymer is characterized by a degree of polymerization of at least 15 and a peptide graft density of 90% to 100%.

102. The peptide brush polymer of claim 87, wherein the peptide brush polymer is in the form of a micelle or nanoparticle.

103. The peptide brush polymer of claim 87, wherein the peptide brush polymer is water-soluble.

104. A pharmaceutical composition comprising the peptide brush polymer according to claim 87.

105. The pharmaceutical composition of claim 104, wherein the pharmaceutical composition comprises an aqueous solution and wherein the peptide brush polymer is in the form of a micelle or nanoparticle in the aqueous solution.

106. A method of administering an effective amount of the peptide brush polymer according to claim 87 or a pharmaceutical composition comprising the same to a subject to treat or manage a condition.


SEQUENCE LISTING (12 sequences)

SEQ ID NO: 1 - protein, 15 aa, synthetic construct
  REGION 1..15: note = Synthetic construct
GPLGLAGGWGERDGS

SEQ ID NO: 2 - protein, 13 aa, synthetic construct
  REGION 1..13: note = Synthetic construct
GALTPRGADSGSG

SEQ ID NO: 3 - protein, 14 aa, synthetic construct
  REGION 1..14: note = Synthetic construct
KLAKLAKKLAKLAK

SEQ ID NO: 4 - protein, 13 aa, synthetic construct
  REGION 1..13: note = Synthetic construct
GSGKEFGADSGSG

SEQ ID NO: 5 - protein, 8 aa, synthetic construct
  REGION 1..8: note = Synthetic construct
GPLGLAGG

SEQ ID NO: 6 - protein, 12 aa, synthetic construct
  REGION 1..12: note = Synthetic construct
HVLVMSATKKKK

SEQ ID NO: 7 - protein, 12 aa, synthetic construct
  REGION 1..12: note = Synthetic construct
GGGCYFQNCPKG

SEQ ID NO: 8 - protein, 8 aa, synthetic construct
  REGION 1..8: note = Synthetic construct
DRVYIHPF

SEQ ID NO: 9 - protein, 9 aa, synthetic construct
  REGION 1..9: note = Synthetic construct
AQYQDKLAR

SEQ ID NO: 10 - protein, 8 aa, synthetic construct
  REGION 1..8: note = Synthetic construct
  SITE 3: note = misc_feature - D-alloisoleucine
GVXSQIRP

SEQ ID NO: 11 - protein, 9 aa, synthetic construct
  REGION 1..9: note = Synthetic construct
KVPRNQDWL

SEQ ID NO: 12 - protein, 12 aa, synthetic construct
  REGION 1..12: note = Synthetic construct
GPLGLAGGWGER